(19) United States
(12) Patent Application Publication
(10) Pub. No.: US 2003/0073862 A1
(43) Pub. Date: Apr. 17, 2003

(54) Compounds

(76) Inventors: Anna-Lena Gustavsson, Stockholm (SE); Lena Jendeberg, Solna (SE); Patrick Roussel, Varese (IT); Martin Slater, Cranbrook (GB); Markus Thor, Knivsta (SE)

Correspondence Address: JEFFREY D. HSI, Fish & Richardson P.C., 225 Franklin Street, Boston, MA, 02110-2804, US

(21) Appl. No.: 10/190,280

(22) Filed: Jul. 3, 2002

Related U.S. Application Data
(60) Provisional application No. 60/304,706, filed on Jul. 11, 2001.

(30) Foreign Application Priority Data
Jul. 3, 2001 (SE) 0102384-5

(51) Int. Cl. C07C 323/29; C07C 255/50; C07C 235/84

(52) U.S. Cl. 558/416; 562/434; 562/455

(57) ABSTRACT

The present invention relates to 2-(benzoylamino)benzoic acid derivatives of the formula I

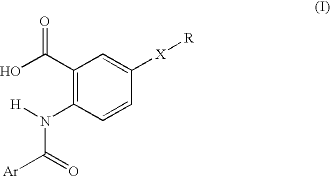

wherein the variants Ar, X and R are as described in the specification. The said compounds modulate the activity of peroxisome proliferator-activated receptors (PPAR) α and/or γ, and are predicted to be useful in the treatment of metabolic diseases, e.g. type II diabetes.

### RELATED APPLICATIONS

This application claims priority to Swedish application No. 0102384-5, filed on Jul. 3, 2001, and U.S. provisional application No. 60/304,706, filed on Jul. 11, 2001, the contents of which are incorporated herein by reference.

### TECHNICAL FIELD

The present invention relates to novel compounds which are 2-(benzoylamino)benzoic acids and which modulate the activity of peroxisome proliferator-activated receptors (PPAR) α and/or γ. The said compounds are predicted to be useful in the treatment of metabolic diseases, e.g. type II diabetes.

### BACKGROUND ART

In developed societies, chronic diseases such as diabetes, obesity, atherosclerosis and cancer are responsible for most deaths. These ailments have complex causes involving genetic, environmental and nutritional factors. There is evidence that a group of closely related nuclear receptors, called peroxisome proliferator-activated receptors (PPARs), may be involved in these diseases. This, together with the fact that PPAR activity can be modulated by drugs such as thiazolidinediones and fibrates, has instigated a huge research effort into PPARs. For reviews on PPARs and their medical significance, see e.g. Kersten, S. et al. (2000) Nature 405:421-424; Willson, T.M. et al. (2000) J. Med. Chem. 43:527-550; Vamecq, J. et al. (1999) Lancet 354:141-148.

The PPARs were first cloned as the nuclear receptors that mediate the effects of synthetic compounds called peroxisome proliferators on gene transcription. It soon became clear that eicosanoids and fatty acids can also regulate gene transcription through PPARs. At the molecular level, PPARs act in a similar manner to other nuclear hormone receptors. First, they bind a specific element in the promoter region of target genes. PPAR and some other nuclear hormone receptors bind the promoter only as a heterodimer with the receptor for 9-cis retinoic acid, RXR (retinoid X receptor). Second, they activate transcription in response to binding of the hormone (ligand). For the PPAR:RXR heterodimer, binding of the ligand of either receptor can activate the complex, but binding of both ligands simultaneously is more potent.

Three PPAR isotypes have been identified: α, β (also called δ and NUCI) and γ. PPARα (GenBank Accession No. NM 005036) is expressed most in brown adipose tissue and liver, then kidney, heart and skeletal muscle. PPARγ (GenBank Accession No. X90563) is mainly expressed in adipose tissue, and to a lesser extent in colon, the immune system and the retina. PPARβ is found in many tissues but the highest expression is in the gut, kidney and heart.

PPARs are ligand-dependent transcription factors: activation of target gene transcription depends on the binding of the ligand to the receptor. Some ligands are shared by the three isotypes, such as polyunsaturated fatty acids and probably oxidized fatty acids.

There are two varieties of diabetes. Type I is insulin-dependent diabetes mellitus (IDDM), for which insulin injection is required; it was formerly referred to as juvenile onset diabetes. In this type, insulin is not secreted by the pancreas and hence must be taken by injection. Type II, non-insulin-dependent diabetes mellitus (NIDDM) may be controlled by dietary restriction. It derives from insufficient pancreatic insulin secretion and tissue resistance to secreted insulin, which is complicated by subtle changes in the secretion of insulin by the beta cells. Despite their former classifications as juvenile or adult, either type can occur at any age; NIDDM, however, is the most common type, accounting for 90 percent of all diabetes.

While the exact causes of diabetes remain obscure, it is evident that NIDDM is linked to heredity and obesity. NIDDM is almost invariably accompanied by dyslipidemia, characterized by elevated triglycerides (TGs), VLDL-C and increased small dense LDL-C in combination with decreased levels of HDL-C and prolonged post-prandial hyperlipidemia. This form of dyslipidemia is highly atherogenic and thus represents a major risk factor for the development of premature atherosclerosis and coronary artery disease (CAD), which is the major cause of mortality in diabetic patients. A direct correlation between low HDL levels and incidence of CAD has been identified. In addition, this pathological lipid profile or “lipotoxicity” is suggested to contribute to P-cell failure and as a consequence impaired glucose stimulated insulin release.

Pharmacological, genetic and biochemical studies have unequivocally established that PPARα and PPARγ are key sensors and transcriptional modulators of lipid and glucose homeostasis, respectively. Accordingly, a selective “dual action drug” that selectively binds and activates PPARα and γ is hypothesized to mechanistically target the two major metabolic abnormalities observed in type II diabetic patients and thus therapeutically intervene with insulin resistance, CAD and possibly also impaired insulin secretion or β-cell failure.

Murakami et al. (1998) Diabetes 47: 1841-1847, discloses a thiazolidinedione derivative which activated both PPARα and PPARγ, and restored reduced lipid oxidation, when administered to obese rats. It was suggested that PPARα agonism has a protective effect against abnormal lipid metabolism in liver of obese rats. Agents modulating both PPARα and PPARγ are also disclosed in Shibata, T. et al. (1999) Eur. J. Pharmacol. 364: 211-219; and in WO 99/19313.

### BRIEF DESCRIPTION OF THE DRAWING

FIG. 1 shows the structure of the ligand-binding domain of human PPARγ, in complex with the compound according to Example 1 of the invention.

### DISCLOSURE OF THE INVENTION

It has surprisingly been found that compounds of the general formula I, which are substituted derivatives of 2-(benzoylamino)benzoic acid, exhibits activity as modulators of peroxisome proliferator-activated receptors (PPAR) α and γ (PPAR modulators). The term “PPAR modulator” is intended to mean a PPAR ligand that is capable of acting as an activator (agonist), or alternatively as an inhibitor (antagonist), in PPAR mediated transcriptional responses.

Consequently, in a first aspect this invention provides a compound of the formula I

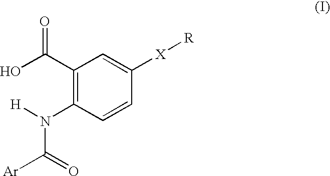

or a pharmaceutically acceptable salt or a prodrug form thereof, wherein

Ar is aryl, which is optionally substituted in one or more positions by

halogen,

cyano,

nitro,

C₁₆ alkyl,

C₁₆ alkoxy,

C₁₆ alkylthio

fluoromethyl,

difluoromethyl,

trifluoromethyl,

difluoromethoxy,

trifluoromethoxy,

difluoromethylthio,

trifluoromethylthio,

allyloxy,

aryloxy, or

arylthio;

X is

a bond, or

a heteroalkyl chain comprising from 1 to 4 carbon atoms and from 1 to 4 heteroatoms,

or

a formula

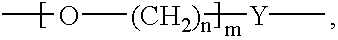

wherein m is O, 1, or 2,

n is 0, 1, 2, or 3, and

Y is a bond, O, S, NH, NHSO2, NHC(O)NH, or CH═CH; and

R is a C₁-C₆-alkyl or an optionally substituted aryl or heteroaryl group,

provided that

when X is a bond, or the formula

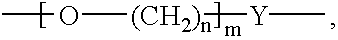

then R is an optionally substituted aryl or heteroaryl group; and

when X is a heteroalkyl chain comprising from 1 to 4 carbon atoms and from 1 to 4 heteroatoms, then R is a C₁-C₆-alkyl or an optionally substituted aryl or heteroaryl group,

with the proviso that

when X is a bond, then R is not a C₁-C₆-alkyl; or

said compound is not a dibenzoyl-bisanthranilic acid, or (4,4′-bis[(1-naphthalenylcarbonyl)amino]-[1,1′-Biphenyl]-3,3′-dicarboxylic acid.

Preferred compounds of the formula I include those wherein:

Ar is phenyl or naphthyl, optionally substituted in one or more positions independently by halogen, nitro, cyano, methoxy, or trifluoromethyl.

X is

a bond;

O—(CH₂)ₙ wherein n is an integer 0 to 3, e.g. O, O—CH₂, or O—(CH₂)₂;

O—(CH₂)ₙ—Y, wherein n is an integer 0 to 3, and Y is an atom selected from O, N

and S, e.g. O(CH₂)₂—O, or O—CH₂)₂—S;

O—(CH₂)₂—O—(CH₂)₂—NH;

O—(CH₂)₂—O—(CH₂)₂—NHSO₂; or

O—(CH₂)₂—O—(CH₂)₂—NHCONH.

R is methyl or selected from the group consisting of, optionally substituted, phenyl, naphthyl, thienyl, pyridinyl, quinoxalinyl, benzoylphenyl, thiazolyl, furyl, imidazolyl, oxazolyl, pyrazinyl, quinolinyl, indolyl, benzofuran, benzothiophenyl (benzothienyl), pyrimidinyl, benzodioxolyl, with the proviso that when X is a bond then R is not methyl

When R is an aryl or heteroaryl, it is independently substituted in one or more positions with

C₁₋₆-alkyl,

C₁₋₆-alkoxy,

C₁₋₆-alkylthio,

C₁₋₆-acyl,

cyano,

nitro,

hydroxy,

methylhydroxy,

carboxy,

fluoromethyl,

difluoromethyl,

trifluoromethyl,

difluoromethoxy,

trifluoromethoxy,

difluoromethylthio,

trifluoromethylthio,

halogen,

formyl,

amino,

C₁₋₆-alkylamino,

di(C₁₋₆-alkyl)amino or C₁₋₆-acylamino,

aryl,

aryloxy,

arylthio,

C₁₋₆-alkylsulphonyl,

C₂₋₆-allyloxy,

benzyloxy,

benzoyl.

In particular, R can be independently substituted in one or more positions with

methyl,

ethyl,

isopropyl,

methoxy,

thiomethoxy

ethoxy,

methylsulfonyl,

formyl,

acetyl,

nitro,

cyano,

methylhydroxy,

methylamino,

carboxy,

trifluoromethyl,

trifluoromethoxy,

chloro,

fluoro,

bromo,

iodo,

benzyloxy,

amino,

dimethylamino,

acetylamino,

phenyl,

phenoxy, or

benzoyl.

The following compounds are especially preferred:

2-[(2,4-dichlorobenzoyl)amino]-5-(2-thienylmethoxy)benzoate,

2-[(2,4-dichlorobenzoyl)amino]-5-(3-pyridinylmethoxy)benzoate,

2-[(2,4-dichlorobenzoyl)amino]-5-[2-(3-thienyl)ethoxy]benzoate,

5-[(2-(3-chlorophenyl)ethoxy]-2-[(2,4-dichlorobenzoyl)amino]benzoate,

2-[(2,4-dichlorobenzoyl)amino]-5-[(4-ethoxybenzyl)oxy]benzoate,

2-[(2,4-dichlorobenzoyl)amino]-5-{[3-(dimethylamino)benzyl]oxy}benzoate,

2-[(2,4-dichlorobenzoyl)amino]-5-[2-(3-methylphenyl)ethoxy]benzoate,

2-[(2,4-dichlorobenzoyl)amino]-5-[2-(1-naphthyl)ethoxy]benzoate,

2-[(2,4-dichlorobenzoyl)amino]-5-(2-pyridinylmethoxy)benzoate,

2-[(2,4-dichlorobenzoyl)amino]-5-(2-{[2-(methylsulfanyl)-3-pyridinyl]oxy} ethoxy)benzoate,

2-[(2,4-dichlorobenzoyl)amino]-5-[2-(3-pyridinyloxy)ethoxy]benzoate,

2-[(2,4-dichlorobenzoyl)amino]-5-[2-(2-quinoxalinyloxy)ethoxy]benzoate,

2-[(2,4-dichlorobenzoyl)amino]-5-{2-[2-(methylsulfanyl)phenoxy]ethoxy}benzoate,

5-[2-(2-aminophenoxy)ethoxy]-2-[(2,4-dichlorobenzoyl)amino]benzoate,

5-[2-(4-benzoylphenoxy)ethoxy]-2-[(2,4-dichlorobenzoyl)amino]benzoate,

2-[(2 ,4-dichlorobenzoyl)amino]-5-[2-(2,3,6-trifluorophenoxy)ethoxy]benzoate,

5-[2-([1,1′-biphenyl]-3-yloxy)ethoxy]-2-[(2,4-dichlorobenzoyl)amino]benzoate,

2-[(2,4-dichlorobenzoyl)amino]-5-[2-(phenylsulfanyl)ethoxy]benzoate,

2-[(2,4-dichlorobenzoyl)amino]-5-[2-(4-methyl-1,3-thiazol-5-yl)ethoxy]benzoate,

2-[(2,4-dichlorobenzoyl)amino]-5-(3-furylmethoxy)benzoate,

2-[(2,4-dichlorobenzoyl)amino]-5-[2-(2-thienyl)ethoxy]benzoate,

2-[(2,4-dichlorobenzoyl)amino]-5-[2-(2-methyl-5-nitro-1 H-imidazol-1-yl)ethoxy]benzoate,

2-[(2,4-dichlorobenzoyl)amino]-5-(3-thienylmethoxy)benzoate,

2-[(2,4-dichlorobenzoyl)amino]-5-[2-(2-pyridinylsulfanyl)ethoxy]benzoate,

2-[(2,4-dichlorobenzoyl)amino]-5-[2-(5-methyl-2-phenyl-1 ,3-thiazol-4-yl)ethoxy]benzoate,

2-[(2,4-dichlorobenzoyl)amino]-5-[2-(5-methyl-2-phenyl-1,3-oxazol-4-yl)ethoxy]benzoate,

2-[(2,4-dichlorobenzoyl)amino]-5-[2-(5-ethyl-2-pyridinyl)ethoxy]benzoate,

2-[(2,4-dichlorobenzoyl)amino]-5-[2-(2-methoxyphenoxy)ethoxy]benzoate,

2-[(2,4-dichlorobenzoyl)amino]-5-(4-pyridinylmethoxy)benzoate,

2-[(2,4-dichlorobenzoyl)amino]-5-[3-(3-pyridinyl)propoxy]benzoate,

2-[(2,4-dichlorobenzoyl)aamino]-5-[2-(2-pyridinyl)ethoxy]benzoate,

2-[(2,4-dichlorobenzoyl)amino]-5-[2-(3-methoxyphenyl)ethoxy]benzoate,

2-[(2,4-dichlorobenzoyl)amino]-5-[(3-nitro-2-pyridinyl)oxy]benzoate,

2-[(2,4-dichlorobenzoyl)amino]-5-[(5-nitro-2-pyridinyl)oxy]benzoate,

2-[(2,4-dichlorobenzoyl)amino]-5-{ [5-(trifluoromethyl)-2-pyridinyl]oxy}benzoate,

5-[(6-chloro-2-pyrazinyl)oxy]-2-[(2,4-dichlorobenzoyl)amino]benzoate,

2-[(2,4-dichlorobenzoyl)amino]-5-(2-pyrimidinyloxy)benzoate,

2-[(2,4-dichlorobenzoyl)amino]-5-{[(2E)-3-phenyl-2-propenyl]oxy}benzoate,

2-[(2,4-dichlorobenzoyl)amino]-5-[(3-methoxybenzyl)oxy]benzoate,

2-[(2,4-dichlorobenzoyl)amino]-5-(3-thienyl)benzoate,

2-[(2,4-dichlorobenzoyl)amino]-5-(2,4-dichlorophenyl)benzoate,

2-[(2,4-dichlorobenzoyl)amino]-5-(4-ethylphenyl)benzoic acid,

2-[(2,4-dichlorobenzoyl)amino]-5-(8-quinolinyl)benzoic acid,

5-(1,3-benzodioxol-5-yl)-2-[(2,4-dichlorobenzoyl)amino]benzoic acid,

2-[(2,4-dichlorobenzoyl)amino]-5-(2,4-dimethoxy-5-pyrimidinyl)benzoic acid,

3′-(acetylamino)-4-[(2,4-dichlorobenzoyl)amino][1,1′-biphenyl]-3-carboxylic acid,

4-[(2,4-dichlorobenzoyl)amino]-3′-(trifluoromethoxy)[1,1′-biphenyl]-3-carboxylic acid,

4-[(2,4-dichlorobenzoyl)amino]-3′-ethoxy[1,1′-biphenyl]-3-carboxylic acid,

5-(1-benzofuran-2-yl)-2-[(2,4-dichlorobenzoyl)amino]benzoic acid,

4-[(2,4-dichlorobenzoyl)amino]-3′-(hydroxymethyl)[1,1′-biphenyl]-3-carboxylic acid,

4-[(2,4-dichlorobenzoyl)amino]-3′-formyl[1,1′-biphenyl]-3-carboxylic acid,

2-[(2,4-dichlorobenzoyl)amino]-5-(2-naphthyl)benzoic acid,

4-[(2,4-dichlorobenzoyl)amino]-3′-isopropyl-6′-methoxy[1,1′-biphenyl]-3-carboxylic acid,

4-[(2,4-dichlorobenzoyl)amino]-4′-fluoro[1,1′-biphenyl]-3-carboxylic acid,

2-[(2,4-dichlorobenzoyl)amino]-5-(2-furyl)benzoate,

5-( 1-benzothien-3-yl)-2-[(2,4-dichlorobenzoyl)amino]benzoate,

2-[(2,4-dichlorobenzoyl)amino]-5-(2-formyl-3-thienyl)benzoate,

5-(5-acetyl-2-thienyl)-2-[(2,4-dichlorobenzoyl)amino]benzoate,

2-[(2,4-dichlorobenzoyl)amino]-5-(1H-indol-5-yl)benzoic acid,

5-(3-carboxyphenyl)-2-[(2,4-dichlorobenzoyl)amino]benzoic acid,

2′-(benzyloxy)-4-[(2,4-dichlorobenzoyl)amino][1,1′-biphenyl]-3-carboxylate,

4-[(2,4-dichlorobenzoyl)amino][1,1′-biphenyl]-3-carboxylate,

4-[(2,4-dichlorobenzoyl)amino]-3′-phenyl[1,1′-biphenyl]-3-carboxylate,

4-[(2,4-dichlorobenzoyl)amino]-3′-(trifluoromethyl)[1,1′-biphenyl]-3-carboxylate,

5-(5-chloro-2-thienyl)-2-[(2,4-dichlorobenzoyl)amino]benzoate,

4-[(2,4-dichlorobenzoyl)amino]-4′-phenoxy[1,1′-biphenyl]-3-carboxylate,

4-[(2,4-dichlorobenzoyl)amino]-2′,5′-dimethoxy[1,1′-biphenyl]-3-carboxylate,

3′-(aminomethyl)-4-[(2,4-dichlorobenzoyl)amino][1,1′-biphenyl]-3-carboxylic acid,

2-(2-naphthoylamino)-5-(3-thienyl)benzo ate,

3′-(acetylamino)-4-(2-naphthoylamino)[1,1′-biphenyl]-3-carboxylate

3′-(hydroxymethyl)-4-(2-naphthoylamino)[1,1′-biphenyl]-3-carboxylate,

5-(3-thienyl)-2-{[4-(trifluoromethyl)benzoyl]amino}benzoate,

3′-(acetylamino)-4-{[4-(trifluoromethyl)benzoyl]amino)}[1,1′-biphenyl]-3-carboxylate,

3′-(hydroxymethyl)-4-{[4-(trifluoromethyl)benzoyl]amino }[1,1′-biphenyl]-3-carboxylate,

2-{[3,5-bis(trifluoromethyl)benzoyl]amino}-5-(8-quinolinyl)benzoate,

4-{[3,5-bis(trifluoromethyl)benzoyl]amino}-3′-formyl[1,1′-biphenyl]-3-carboxylate,

2-[(4-methoxybenzoyl)amino]-5-(8-quinolinyl)benzoate,

4-[(3 ,5-dichlorobenzoyl)amino][1,1′-biphenyl]-3-carboxylate,

2-[(4-cyanobenzoyl)amino]-5-(2-thienylmethoxy)benzoate,

2-[(2,4-difluorobenzoyl)amino]-5-(2-thienylmethoxy)benzoate,

2-[(2-chlorobenzoyl)amino]-5-[(3-nitro-2-pyridinyl)oxy]benzoate,

2-[(2-chloro-5-nitrobenzoyl)amino]-5-(2-thienylmethoxy)benzoate, or

2-[(2,4-dichlorobenzoyl)amino]-5-[2-(methylsulfanyl)ethoxy]benzoate.

Definitions

The term “C₁₋₆ alkyl” denotes a straight or branched alkyl group having from 1 to 6 carbon atoms. Examples of said C_{1 6} alkyl include methyl, ethyl, n-propyl, iso-propyl, n-butyl, iso-butyl, sec-butyl, t-butyl and straight-and branched-chain pentyl and hexyl.

The term “C₁₋₆ alkoxy” denotes a straight or branched alkoxy group having from 1 to 6 carbon atoms. Examples of said C₁₋₆ alkoxy include methoxy, ethoxy, n-propoxy, iso-propoxy, n-butoxy, iso-butoxy, sec-butoxy, t-butoxy and straight-and branched-chain pentoxy and hexoxy.

The term “halogen” shall mean fluorine, chlorine, bromine or iodine.

The term “aryl” denotes aromatic rings (monocyclic or bicyclic) having from 6 to 10 ring carbon atoms. Examples of said aryl include phenyl, indenyl and naphthyl.

The term “heteroaryl” denotes a mono-or bicyclic ring system (only one ring need to be aromatic, and substitution may be in any ring) having from 5 to 10 ring atoms (which are carbon atoms), in which one or more of the carbon ring atoms are other than carbon, such as nitrogen, oxygen, selenium, and sulfur. Examples of said heteroaryl include pyrrole, thiazole, imidazole, thiophene, furan, isothiazole, thiadiazole, oxazole, isoxazole, oxadiazole, pyridine, pyrazine, pyrimidine, pyridazine, pyrazole, triazole, tetrazole, chroman, isochroman, quinoline, quinoxaline, isoquinoline, phthalazine, quinazolineindole, indole, isoindole, isoindoline, indoline, benzothiophene, benzofuran, 2,3-dihydrobenzofuran, isobenzofuran, benzoxazole, 2,1,3-benzoxadiazole, benzothiazole, 2,1,3-benzothiadiazole, 2,1,3-benzoselenadiazole, benzimidazole, indazole, 2,3-dihydro-1,4-benzodioxine, indane, 1,3-benzodioxole, 3,4-dihydro-2H-1,4-benzoxazine, 1,5-naphtyridine, 1,8-naphtyridine, 1,5-naphthyridine, and 1,8-naphthyridine.

The term “heteroalkyl chain” denotes a straight or branched, saturated or unsaturated, chain comprising from 1 to 4 carbon atoms and from 1 to 4 heteroatoms selected from the group consisting of O, N, and S. The heteroatom(s) may be placed at any position of the heteroalkyl group.

Depending on the process conditions the end products of the Formula I are obtained either in neutral or salt form (e.g., lithium, sodium, potassium salts, hydrochloride, hydrobromide, and the like). Both the free base and the salts of these end products are within the scope of the invention.

All diastereomeric forms possible (pure enantiomers, racemic mixtures and unequal mixtures of two enantiomers) are within the scope of the invention.

Therapeutic or prophylactic treatment of mammals, including man, for conditions where modulation of either PPARα or PPARγ0 activity, or the combination of both PPARα and PPARγ activities, is of therapeutic benefit. Such conditions could be e.g. diabetes, diabetes mellitus type 2, insulin resistance, impaired glucose tolerance and/or in combinations with dyslipidemias, obesity, atherosclerosis, coronary artery disease, PCOS, gestational diabetes, inflammation.

The compounds according to the invention are particularly useful for the treatment of type II diabetes, in combination(s) with dyslipidemias, obesity, atherosclerosis and coronary artery disease. For this purpose the compounds according to the invention can be used alone or in combination(s) with sulfonylureas, metformin, alpha-glycosidase inhibitors, insulin or other anti-diabetic treatments/agents. Reference to treatment is intended to include prophylaxis as well as the alleviation of established symptoms.

For clinical use, the compounds of the invention are formulated into pharmaceutical formulations for oral, rectal, parenteral or other mode of administration. Pharmaceutical formulations are usually prepared by mixing the active substance, or a pharmaceutically acceptable salt thereof, with conventional pharmaceutical excipients. The formulations can be further prepared by known methods such as granulation, compression, microencapsulation, spray coating, etc.

The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner. The typical daily dose of the active substance vanres within a wide range and will depend on various factors such as for example the individual requirement of each patient and the route of administration.

The compounds according to the invention may also be administered as prodrugs that may be converted to the active ingredient in question after metabolic transformation in vivo. Conventional procedures for the selection and preparation of suitable prodrug derivatives are described, for example, in “Design of Prodrugs” ed. H. Bundgaard, Elsevier, 1985.

This invention also relates to a method of treatment or prevention of diabetes. The method includes administering to a subject (e.g., a human, a mammal, a horse, a dog, or a cat) in need thereof an effective amount of one or more compounds of the formula I:

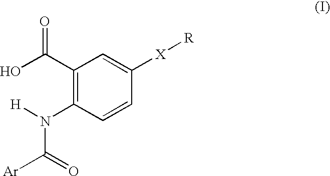

or a pharmaceutically acceptable salt or a prodrug form thereof, wherein

Ar is aryl, which is optionally substituted in one or more positions by

halogen,

cyano,

nitro,

C₁₋₆ alkyl,

C₁₋₆ alkoxy,

C₁₋₆ alkylthio

fluoromethyl,

difluoromethyl,

trifluoromethyl,

difluoromethoxy,

trifluoromethoxy,

difluoromethylthio,

trifluoromethylthio,

allyloxy,

aryloxy, or

arylthio;

X is

a bond, or

a heteroalkyl chain comprising from 1 to 4 carbon atoms and from 1 to 4 heteroatoms,

or

a formula

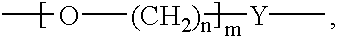

wherein m is 0, 1, or 2,

n is0, 1, 2, or 3, and

Y is a bond, O, S, NH, NHSO2, NHC(O)NH, or CH═CH; and

R is C₁-C₆-alkyl or an optionally substituted aryl or heteroaryl group.

The methods delineated herein can also include the step of identifying that the subject is in need of treatment of diabetes.

Also within the scope of this invention is a method for modulating (e.g., stimulating or inhibiting) peroxisome proliferator-activated receptors activities. The method includes contacting the receptors with an effective stimulatory or inhibitory amount of a compound of the formula I.

“An effective amount” refers to an amount of a compound which confers a therapeutic effect on the treated subject. The therapeutic effect may be objective (i.e., measurable by some test or marker) or subjective (i.e., subject gives an indication of or feels an effect). The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

Processes for preparation

In a further aspect the invention provides a process for the preparation of a compound as defined above. The compounds according to the invention can be prepared by, or in analogy with, standard synthetic methods, and especially according to, or in analogy with, the following methods.

Method 1

Compounds of formula (I) in which X is oxygen can be prepared beginning with commercially available 2-amino-5-hydroxybenzoic acid (i) as shown in Scheme 1. The corresponding methyl ester (ii) is formed by treatment with sulfuric acid and methanol and is subsequently coupled with a benzoyl chloride or a heteroarylcarbonyl chloride (commercially available or prepared from the corresponding carboxylic acid using thionyl chloride or oxalyl chloride) to provide the amide (iii). Reaction of (iii) with an alcohol in the presence of diethyl azodicarboxylate (DEAD) or 1,1′-azobis(N,N-dimethylformamide) (TMAD; cf. Tetrahedron Lett.1995, vol. 36: 3789-3792) and triphenylphosphine or polymer supported triphenylphosphine in a solvent such as dichloromethane and/or tetrahydrofuran (Mitsunobu reaction; see Org. React. 1992, vol. 42: 335-656) gives the adduct (iv). Ester hydrolysis, using 1 M lithium hydroxide, affords the target compounds (v) as lithium salts.

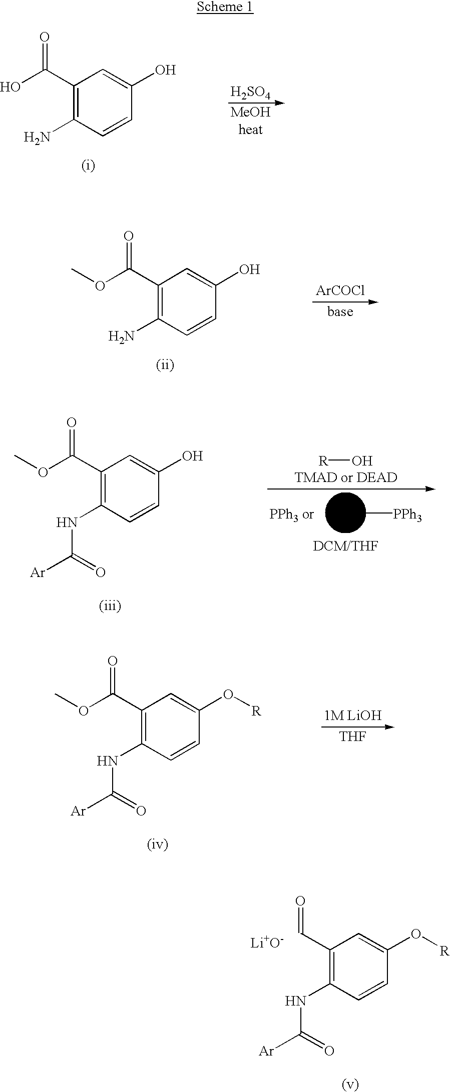

Method 2

Other compounds of the present invention can be prepared as shown in Scheme 2. The Mitsunobu reaction can also be performed on the intermediate (ii), i.e. before the amide coupling, to form the adduct (vi). Subsequent amide coupling and ester hydrolysis afford the target compounds (v).

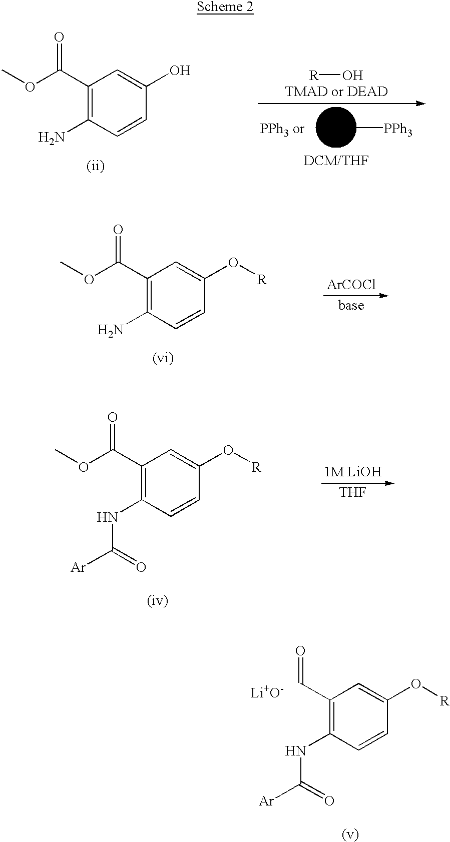

Method 3

Compounds of formula (I) in which X=C₀ and R is an aryl or heteroaryl substituent can be prepared as outlined in Scheme 3. Treatment of the commercially available 2-amino-5-iodobenzoic acid (vii) with trichloromethyl chloroformate in solvents such as dioxane gives the isatoic anhydride (viii) which can be further reacted with methanol and a base such as potassium carbonate to form the methyl ester (ix). Subsequent coupling with a benzoyl chloride or a heteroarylcarbonyl chloride (commercially available or prepared from the corresponding carboxylic acid using thionyl chloride or oxalyl chloride) provides amide (x). Palladium-catalyzed cross-coupling of (x) with an aryl or heteroaryl boronic acid (Suzuki coupling; see Chem. Rev. 1995, 95, 2457-2483) gives biaryl (xii) or a mixture of (xii) and the bicycle (xi). Subsequent ester hydrolysis using 1M lithium hydroxide solution affords the target compounds (xiii).

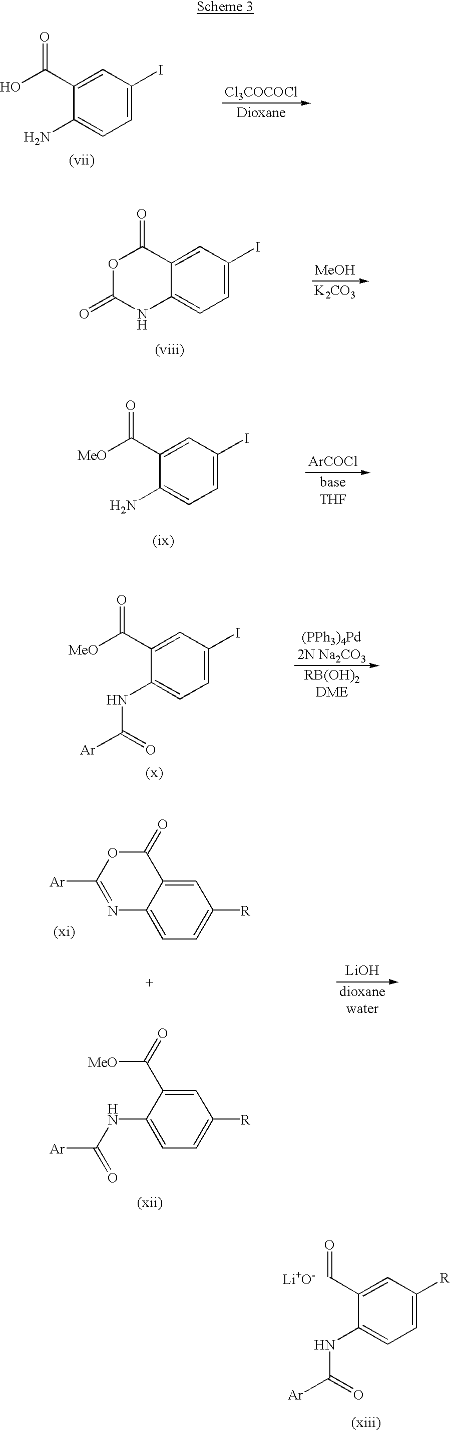

Method 4

Other compounds of the present invention can be prepared as shown in Scheme 4. The intermediate (iii) can be reacted with nitrogen containing heterocycles to form diaryl ethers (xiv) which can be hydrolyzed as described earlier to afford compounds (xv).

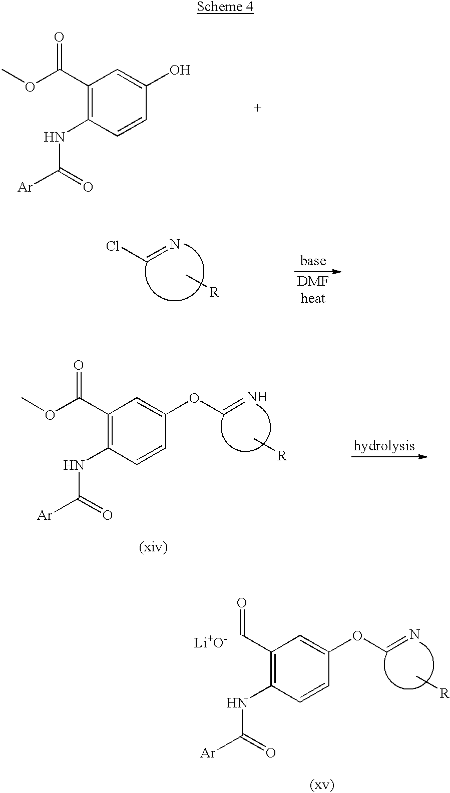

Method 5

Other compounds of the present invention can be prepared as shown in Scheme 5. Intermediate (iii) can be reacted with benzylic (or aliphatic) bromides to form compounds (xvi) which can be hydrolyzed as described earlier to afford compounds (xvii).

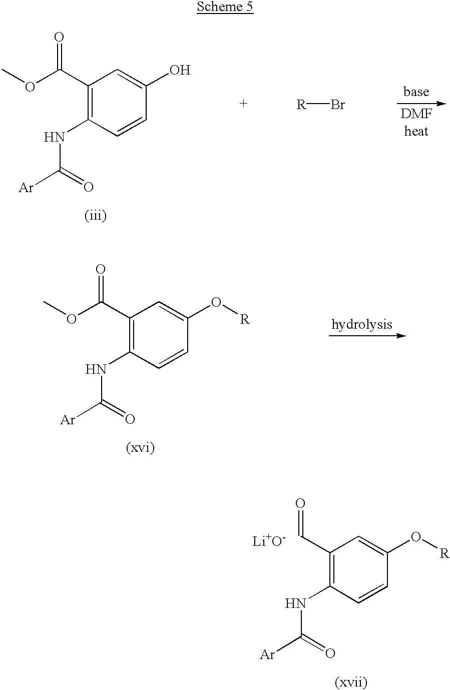

The chemicals used in the above-described synthetic routes may include, for example, solvents, reagents, catalysts, protecting group and deprotecting group reagents. The methods described above may also additionally include steps, either before or after the steps described specifically herein, to add or remove suitable protecting groups in order to ultimately allow synthesis of the compounds of Formula (I). In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 2^{nd} Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The invention will now be further illustrated by the following non-limiting examples. Without further elaboration, it is believed that one skilled in the art can, based on the description herein, utilize the present invention to its fullest extent. All publications cited herein are hereby incorporated by reference in their entirety.

### EXAMPLES

The structures of the prepared compounds were confirmed by standard spectroscopical methods. The NMR data was obtained on a Jeol JNM-EX270 or a Bruker DRX500 spectrometer. Electrospray MS data was obtained on a Micromass platform LCMS spectrometer. Melting points, when given, were obtained on a Electrothermal IA9000 melting point apparatus, and are uncorrected.

### Example 1

Lithium 2-[(2,4-dichlorobenzoyl)amino]-5-(2-thienylmethoxy)benzoate

Step 1: Methyl 2-amino-5-hydroxybenzoate

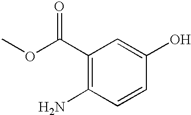

To a stirred suspension of 2-amino-5-hydroxybenzoic acid (15 g, 98 mmol) in methanol (100 ml) was added sulfuric acid (95%, 15 ml) at room temperature. The solution was stirred at 90° C. for 3.5 hours after which it was allowed to reach room temperature and carefully poured into saturated sodium bicarbonate. Subsequent extraction with chloroform (3×300 ml), drying of the organic phase using magnesium sulfate and concentration in vacuo gave the title compound (15 g, 80%) as a dark solid. mp: 154-155° C.; ¹H NMR (DMSO) δ 5 8.66 (s, 1H), 7.09 (d, J=2.72 Hz 1H), 6.82-6.76 (m, 1H), 6.66-6.60 (m, 1H), 6.07 (br s, 2H), 3.75 (s, 3H); ¹³C NMR (DMSO) δ 167.7, 146.6, 144.8, 123.6, 117.9, 114.4, 108.8, 51,4; MS m/z 168 (M+1).

Step 2: Methyl 2-[(2,4-dichlorobenzoyl)amino]-5-hydroxybenzoate

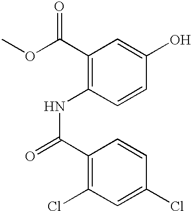

To a stirred mixture of methyl 2-amino-5-hydroxybenzoate (10 g, 60 mmol) pyridine (80 ml) and molecular sieves (4 Å), 2,4-dichlorobenzoyl chloride (7.6 ml, 54 mmol) in pyridine (3 ml) was added slowly at 0° C. The mixture was allowed to reach room temperature and then stirred over night. After addition of chloroform, the mixture was filtered and the filtrate washed with 1M hydrochloric acid (3×150 ml) and brine, dried with magnesium sulfate and concentrated in vacuo. The residue was re-crystallized from chloroform to give the title compound (4 g, 20%) as a grey solid. mp: 181-182° C.; ¹H NMR (DMSO) ( 10.64 (s, 1H), 9.81 (s, 1H), 7.92-7.55 (m, 4H), 7.29 (d, J=2.73 Hz 1H), 7.08-7.02 (m, 1H), 3.79 (m, 3H); MS m/z 338 (M−1).

Step 3: Methyl 2-[(2,4-dichlorobenzoyl)amino]-5-(2-thienylmethoxy)benzoate General procedure A

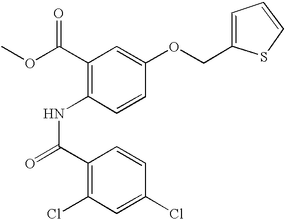

TMAD (183 mg, 1.06 mmol) was added to a suspension of methyl 2-[(2,4-dichlorobenzoyl)amino]-5-hydroxybenzoate (240 mg, 0.71 mmol; prepared in Example XX), polymer bound triphenylphosphine (480 mg, 1.4 mmol) and thiophene-2-methanol (73 μl, 0.78 mmol) in anhydrous THF (3 ml) and DCM (3 ml). The suspension was shaken at room temperature over night and filtered through a plug of Celite. The filtrate was concentrated in vacuo and the residue purified by chromatography on silica gel eluting with CHCl₃ to give the title compound (130 mg, 42%) as an yellow oil. ¹H NMR (CDCl₃) S 11.31 (s, 1H), 8.79 (d, J=9.40 Hz 1H), 7.67-7.57 (m, 2H), 7.47 (d, J=1.98 Hz 1H), 7.35-7.30 (m, 2H), 7.27-7.21 (m, 1H), 7.12-7.09 (m, 1H), 7.02-6.97 (m, 1H), 5.23 (s, 1H), 3.89 (s, 3H); ¹³C NMR (CDCl₃) δ 168.3, 164.1, 153.7, 138.7, 136.9, 135.2, 134.7, 132.3, 130.5, 130.4, 127.6, 127.2, 127.0, 126.6, 122.2, 122.0, 116.6, 166.6, 65.5, 52.7

Step 4: Lithium 2-[(2,4-dichlorobenzoyl)amino]-5-(2-thienylmethoxy)benzoate General procedure B

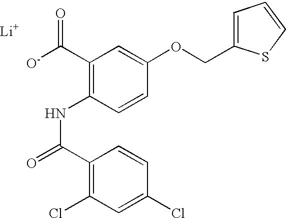

Lithium hydroxide (1 M solution, 298 μl) was added at room temperature to a stirred solution of methyl 2-[(2,4-dichlorobenzoyl)amino]-5-(2-thienylmethoxy)benzoate (130 mg, 0.30 mmol) in THF (2 ml). The mixture was stirred over night and then concentrated in vacuo, re-dissolved in methanol and concentrated again. The residue was washed with diethyl ether to give the title compound (120 mg, 94%) as an yellow solid. mp: 165-168° C.; ¹H NMR (CD₃OD) δ 8.56 (d, J=8.91 Hz 1H), 7.75 (d, J=2.97 Hz 1H), 7.66-7.56 (m, 2H), 7.47-7.37 (m, 2H), 7.17-7.13 (m, 1H), 7.08 (dd, J=9.16, 3.22 Hz 1H), 7.02-6.97 (m, 1H), 5.27 (s, 2H); ¹³C NMR (CD₃OD) δ 172.5, 164.4, 154.1, 139.6, 136.2, 135.6, 133.6, 132.1, 129.9, 127.4, 126.7, 126.3, 125.9, 125.6, 120.6, 118.0, 116.9,64.9; MS m/z 420 (M−1).

### Example 2

Lithium 2-[(2,4-dichlorobenzoyl)amino]-5-(3-pyridinylmethoxy)benzoate

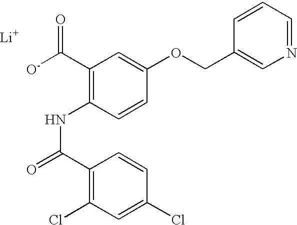

Step 1: Methyl 2-[(2,4-dichlorobenzoyl)amino]-5-(3-pyridinylmethoxy)benzoate

Use of pyridine-3-methanol afforded the title compound (227 mg, 75%) as a white solid by the application of the general procedure A described above. ¹H NMR (CDCl₃) δ 11.27 (s, 1H), 8.75 (d, J=9.40 Hz 1H), 8.65 (d, J=1.73 Hz 1H), 8.55 (dd, J=1.73, 4.70 Hz 1H), 7.76-7.70 (m, 1H), 7.61-7.53 (m, 2H), 7.42-7.40 (m, 1H), 7.31-7.16 (m, 3H), 5.05 (s, 2H), 3.85 (s, 3H); ¹³C NMR (CDCl₃) δ 168.1, 164.1, 153.7, 149.7, 149.1, 136.9, 135.4, 135.2, 134.6, 132.3, 132.1, 130.5, 127.6, 123.6, 122.3, 121.7, 116.7, 116.1, 68.1, 52.7.

Step 2: Lithium 2-[(2,4-dichlorobenzoyl)amino]-5-(3-pyridinylmethoxy)benzoate

Use of methyl 2-[(2,4-dichlorobenzoyl)amino]-5-(3-pyridinylmethoxy)benzoate afforded the title compound (227 mg, 75%) as a solid by the application of the general procedure B described in Example 1. ¹H NMR (CD₃OD) δ 8.66-8.46 (m, 3H), 7.98-7.92 (m, 1H), 7.77 (d, J=2.97 Hz 1H), 7.65-7.55 (m, 2H), 7.49-7.40 (m, 2H), 7.11 (dd, J=9.16, 3.21 Hz 1H), 5.17 (s, 2H); ¹³C NMR (CD₃OD) δ 172.4, 164.4, 154.1, 148.2, 148.0, 136.4, 136.2, 135.6, 134.0, 133.8, 132.1, 129.9, 129.8, 127.4,125.7, 124.0, 120.7, 117.8, 116.7,67.3;MS m/z 415 (M−1).

### Example 3

Lithium 2-[(2,4-dichlorobenzoyl)amino]-5-12-(3-thienyl)ethoxy)benzoate

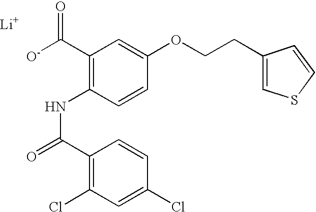

Step 1: Methyl 2-[(2,4-dichlorobenzoyl)amino]-5-[2-(3-thienyl)ethoxy]benzoate

Use of 2-(3-thienyl)ethanol afforded the title compound (370 mg, 93%) as an oil by the application of the general procedure A described above. ¹H NMR (CDCl₃) δ 11.31 (s, 1H), 8.78 (d, J=9.15 Hz 1H), 7.62-7.53 (m, 2H), 7.45 (d, J=1.98 Hz 1H), 7.34-7.25 (m, 2H), 7.19-7.13 (dd, J=9.16, 3.21 Hz 1H), 7.10-7.07 (m, 1H), 7.05-7.01 (m, 1H), 4.17 (t, J=6.92 Hz 2H), 3.87 (s, 3H), 3.12 (t, J=6.93 Hz); ¹³C NMR (CDCl₃) δ 168.3, 164.1, 154.3, 138.3, 136.9, 134.8, 132.4, 130.5, 130.4, 128.5, 127.6, 125.8, 122.3, 121.8, 121.6, 116.6, 115.7, 68.6, 52.7, 30.3.

Step 2: Lithium 2-[(2,4-dichlorobenzoyl)amino]-5-[2-(3-thienyl)ethoxy]benzoate

Use of methyl 2-[(2,4-dichlorobenzoyl)amino]-5-[2-(3-thienyl)ethoxy]benzoate afforded the title compound (260 mg, 95%) as a white solid by the application of the general procedure B described above. ¹H NMR (CD₃OD) δ 8.55 (d, J=8.91 Hz 1H), 7.69-7.55 (m, 5 3H), 7.43 (dd, J=8.16, 1.97 Hz 1H), 7.34-7.29 (m, 1H), 7.18-7.15 (m, 1H), 7.09-6.98 (m, 2H), 4.20 (t, J=6.68 Hz 2H), 3.09 (t, J=6.68 Hz 2H); ¹³C NMR (CD₃OD) δ 172.7, 164.3, 154.6, 138.8, 136.1, 135.7, 133.2, 132.1, 129.8, 128.2, 127.4, 125.6, 125.0, 121.1, 120.6, 117.5, 116.3, 68.2, 29.9; MS m/z 434 (M−1).

### Example 4

Lithium 5-[2-(3-chlorophenyl)ethoxy]-2-[(2,4-dichlorobenzoyl)amino]benzoate

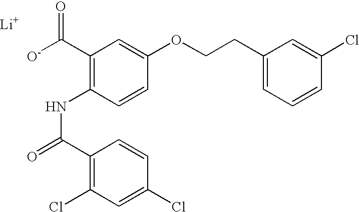

Step 1: Methyl 5-[2-(3-chlorophenyl)ethoxy]-2-[(2,4-dichlorobenzoyl)amino]benzoate

Use of 3-chlorophenethyl alcohol afforded the title compound (342 mg, 60%) as a white solid by the application of the general procedure A described above. ¹H NMR (CDCl₃) δ 11.30 (s, 1H), 8.77 (d, J=9.15 Hz 1H), 7.61-7.45 (m, 3H), 7.35-7.12 (m, 7H), 4.17 (t, J=6.93 Hz 2H), 3.88 (s, 3H), 3.06 (t, J=6.68 Hz 2H); ¹³C NMR (CDCl₃) δ 168..3, 164.1, 154.1, 140.2, 136.9, 134.9, 134.7, 134.3, 132.3, 130.5, 130.4, 129.9, 129.2, 127.6, 127.3, 126.9, 122.3, 121.6, 116.6, 115.7, 68.8, 52.7, 35.5; MS m/z 480 (M+1).

Step 2: Lithium 5-[2-(3-chlorophenyl)ethoxy]-2-[(2,4-dichlorobenzoyl)amino]benzoate

Use of methyl 5-[2-(3-chlorophenyl)ethoxy]-2-[(2,4-dichlorobenzoyl)amino]benzoate afforded the title compound (100 mg, 91 %) as a white solid by the application of the general procedure B described above. ¹H NMR (CD₃OD) δ 8.54 (d, J=8.91 Hz 1H), 7.66-7.55 (m, 3H), 7.46-7.41 (m, 1H), 7.35 (br s, 1H), 7.29-7.17 (m, 3H), 7.00 (dd, J=9.16, 3.22 Hz 1H), 4.22 (t, J=6.68 Hz 2H), 3.07 (t, J=6.68 Hz 2H); MS m/z 462 (M−1); Anal. (C₂₂H₁₅Cl₃LiNO₄) C, H, N.

### Example 5

Lithium 2-[(2,4-dichlorobenzoyl)amino]-5-[(4-ethoxybenzyl)oxy]benzoate

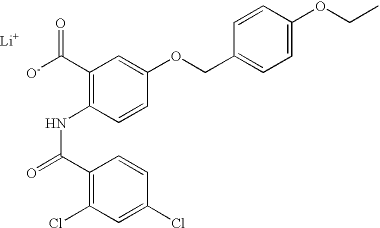

Step 1: Methyl 2-[(2,4-dichlorobenzoyl)amino]-5-[(4-ethoxybenzyl)oxy]benzoate

Use of 4-ethoxybenzyl alcohol afforded the title compound (206 mg, 37%) as an yellow solid by the application of the general procedure A described above. ¹H NMR (CDCl₃) δ 11.30 (s, 1H), 8.78 (d, J-=9.15 Hz 1H), 7.65-7.57 (m, 2H), 7.47 (d, J=1.98 Hz 1H), 7.37-7.30 (m, 3H), 7.25-7.20 (m, 1H), 6.93-6.87 (m, 2H), 4.99 (s, 2H), 4.03 (q, J=6.93 Hz 2H), 3.89 (s, 3H), 1.41 (t, J=7.05 Hz 3H); ¹³C NMR (CDCl₃) δ 168.4, 164.1, 159.0, 154.3, 136.9, 134.8, 132.3, 130.5, 130.4, 129.4, 128.3, 127.6, 122.2, 121.9, 116.6, 116.3, 114.7, 70.4, 63.6, 52.7, 14.9; MS m/z 474 (M+1).

Step 2: Lithium 2-[(2,4-dichlorobenzoyl)amino]-5-[(4-ethoxybenzyl)oxy]benzoate

Use of methyl 2-[(2,4-dichlorobenzoyl)amino]-5-[(4-ethoxybenzyl)oxy]benzoate afforded the title compound (110 mg, 92%) as an yellow solid by the application of the general procedure B described above. ¹H NMR (CD₃OD) δ 8.54 (d, J=8.90 Hz 1H), 7.74 (d, J=2.97 Hz 1H), 7.66-7.55 (m, 2H), 7.47-7.31 (m, 3H), 7.06 (dd, J=9.16, 3.22 Hz 1H), 6.93-6.86 (m, 2H), 5.01 (s, 2H), 4.06-3.95 (m, 2H), 1.37 (t, J=6.93 Hz 3H); MS m/z 458 (M−1); Anal. (C₂₃H₁₈Cl₂LiNO₅) C, H, N.

### Example 6

Lithium 2-1[(2,4-dichlorobenzoyl)amino]-5-{[3-(dimethylamino)benzyl]oxy}benzoate

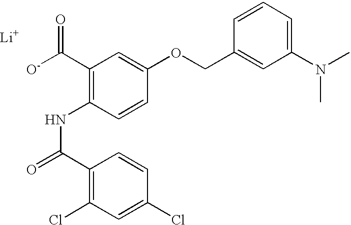

Step 1: Methyl 2-[(2,4-dichlorobenzoyl)amino]-5-([3-(dimethylamino)benzyl]oxyybenzoate

Use of 3-dimethylbenzyl alcohol afforded the title compound (385 mg, 69%) as an yellow oil by the application of the general procedure A described above. ¹H NMR (CDCl₃) δ 11.31 (s, 1H), 8.79 (d, J=9.16 Hz 1H), 7.69-7.58 (m, 2H), 7.48 (d, J=1.98 Hz 1H), 7.37-7.22 (m, 3H), 6.81-6.67 (m, 3H), 5.05 (s, 2H), 3.89 (s, 3H), 2.96 (s, 3H); ¹³C NMR (CDCl₃) δ 168.4, 164.1, 154.4, 150.9, 137.3, 136.9, 134.8, 132.3, 130.5, 129.5, 127.6, 122.2, 121.9, 116.6, 116.3, 115.8, 112.4, 111.6, 71.1, 52.7, 40.7; MS m/z 471 (M−1).

Step 2: Lithium 2-[(2,4-dichlorobenzoyl)amino]-5-{[3-(dimethylamino)benzyl]oxyybenzoate

Use of methyl 2-[(2,4-dichlorobenzoyl)amino]-5-{[3-(dimethylamino)benzyl]oxy}benzoate afforded the title compound (100 mg, 65%) as an yellow solid by the application of the general procedure B described above. ¹H NMR (CD₃OD) δ 8.54 (d, J=8.90 Hz 1H), 7.75 (d, J=3.21 Hz 1H), 7.67-7.56 (m, 2H), 7.47-7.41 (m, 1H), 7.19 (t, J=7.92 Hz 1H), 7.07 (dd, J=9.15, 3.21 Hz 1H), 6.89-6.69 (m, 3H), 5.05 (s, 2H), 2.92 (s, 6H); MS m/z 457 (M−1); Anal. (C₂₃H₁₉Cl₂LiN₂O₄) C, H, N.

### Example 7

Lithium 2-1[(2,4-dichlorobenzoyl)amino]-5-[2-(3-methylphenyl)ethoxy]benzoate

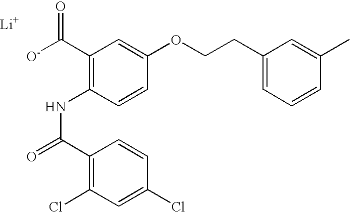

Step 1: Methyl 2-[(2,4-dichlorobenzoyl)amino]-5-[2-(3-methylphenyl)ethoxy]benzoate

Use of 3-methylphenethyl alcohol afforded the title compound (362 mg, 67%) as an oil by the application of the general procedure A described above. ¹H NMR (CDCl₃) δ 11.32 (s, 1H), 8.79 (d, J=9.15 Hz 1H), 7.63-7.54 (m, 2H), 7.48 (d, J=1.98 Hz 1H), 7.36-7.32 (m, 1H), 7.27-7.05 (m, 5H), 4.18 (t, J=7.18 Hz 2H), 3.89 (s, 3H), 3.08 (t, J=7.18 Hz 2H), 2.36 (s, 3H); ¹³C NMR (CDCl₃) δ 168.4, 164.1, 154.4, 138.3, 137.9, 136.9, 134.7, 132.4, 130.5, 130.4, 129.9, 128.6, 127.6, 127.5, 126.1, 122.2, 121.7, 116.6, 115.6, 69.4, 52.6, 35.8, 21.5; MS m/z 458 (M+1).

Step 2: Lithium 2-[(2,4-dichlorobenzoyl)amino]-5-[2-(3-methylphenyl)ethoxy]benzoate

Use of methyl 2-[(2,4-dichlorobenzoyl)amino]-5-[2-(3-methylphenyl)ethoxy]benzoate afforded the title compound (119 mg, 94%) as a solid by the application of the general procedure B described above. 1H NMR (CD₃OD) δ 8.53 (d, J=9.16 Hz 1H), 7.67-7.56 (m, 3H), 7.46-7.41 (m, 1H), 7.20-6.96 (m, 5H), 4.19 (t, J=6.93 Hz 2H), 3.03 (t, J=6.93 Hz 2H), 2.31 (s, 3H); ¹³C NMR (CD₃OD) δ 164.3, 154.7, 138.4, 137.7, 136.1, 135.7, 133.2, 132.1, 129.9, 129.8, 129.4, 128.0, 127.4, 126.7, 125.8, 125.6, 120.6, 117.5, 68.9, 35.4, 20.2; MS m/z 448 (M−1).

### Example 8

Lithium 2-[(2,4-dichlorobenzoyl)amino]-5-[2-(1-naphthyl)ethoxy]benzoate

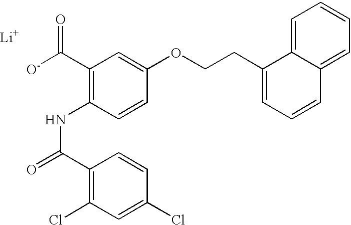

Step 1: Methyl 2-[(2,4-dichlorobenzoyl)amino]-5-[2-(]-naphthyl)ethoxyfbenzoate Use of 2-(1-naphthyl)ethanol afforded the title compound (417 mg, 71%) as an yellow oil by the application of the general procedure A described above. ¹H NMR (CDCl₃) δ 11.32 (s, 1H), 8.80 (d, J=9.15 Hz 1H), 8.14-8.09 (m, 1H), 7.92-7.76 (m, 2H), 7.63-7.43 (m, 7H), 7.36-7.31 (m, 1H), 7.21-7.15 (m, 1H), 4.32 (t, J=7.42 Hz 2H), 3.87 (s, 3H), 3.60 (t, J=7.42 Hz 2H); ¹³C NMR (CDCl₃) δ 168.3, 164.1, 154.3, 136.9, 134.8, 134.0, 130.5, 130.4, 129.0, 127.6, 127.3, 126.3, 125.8, 125.7, 123.6, 122.3, 121.8, 116.6, 115.4, 68.6, 52.6, 32.9; MS m/z 494 (M+1).

Step 2: Lithium 2-[(2,4-dichlorobenzoyl)amino]-5-[2-(1-naphthyl)ethoxy]benzoate

Use of methyl 2-[(2,4-dichlorobenzoyl)amino]-5-[2-(1-naphthyl)ethoxy]benzoate afforded the title compound (140 mg, 79%) as a white solid by the application of the general procedure B described above. ¹H NMR (CD₃OD) δ 8.53 (d, J=8.91 Hz 1H), 8.16 (d, J=8.66 Hz 1H), 7.89-7.36 (m, 10H), 6.99 (dd, J=9.15, 3.21 Hz 1H), 4.34 (t, J=6.93 Hz 2H), 3.57 (t, J=6.93 Hz 2H); MS m/z 478 (M−1).

### Example 9

Lithium 2-[(2,4-dichlorobenzoyl)amino]-5-(2-pyridinylmethoxy)benzoate

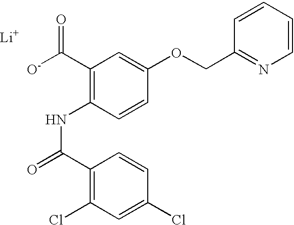

Step 1: Methyl 2-[(2,4-dichlorobenzoyl)amino]-5-(2-pyridinylmethoxy)benzoate

Use of pyridine-2-methanol afforded the title compound (58 mg, 27%), as a white solid, by the application of the general procedure A described above. ¹H NMR (CDCl₃) δ 11.31 (s, 1H), 8.81 (d, J=9.42 Hz 1H), 8.66-8.60 (m, 1H), 7.78-7.68 (m, 2H), 7.62 (d, J=8.16 Hz 1H), 7.55-7.48 (m, 2H), 7.37 (d, J=8.17 Hz 1H), 7.32-7.23 (m, 2H), 5.25 (s, 2H), 3.91 (s, 3H); MS m/z 431 (M+1).

Step 2: Lithium 2-[(2,4-dichlorobenzoyl)amino]-5-(2-pyridinylmethoxy)benzoate

Use of methyl 2-[(2,4-dichlorobenzoyl)amino]-5-(2-pyridinylmethoxy)benzoate afforded the title compound (31 mg, 93%) as a white solid by the application of the general procedure B described above. ¹H NMR (DMSO) δ 14.95 (s, 1H), 8.78-6.88 (m, 10H), 5.15 (s, 2H); MS m/z 415 (M−1).

### Example 10

Lithium 2-[(2,4-dichlorobenzoyl)amino]-5-(2-{[2-(methylsulfanyl)-3-pyridinyl]oxy}ethoxy)benzoate

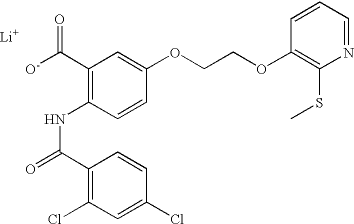

Step 1: Methyl 2-[(2,4-dichlorobenzoyl)amino]-5-(2-{[2-(methylsulfanyl)-3-pyridinyl]oxy ethoxy)benzoate

Use of 2-{[2-(methylsulfanyl)-3-pyridinyl]oxy}ethanol (disclosed in WO 00/76984) afforded the title compound (104 mg, 41%), as a white solid, by the application of the general procedure A described above. ¹H NMR (CDCl₃) δ 11.29 (s, 1H), 8.80 (d, J=9.42 Hz 1H), 8.13 (d, J=4.71 Hz 1H), 7.67-7.61 (m, 2H), 7.52-7.50 (m, 1H), 7.39-7.36 (m, 1H), 7.29-7.26 (m, 1H), 7.10-7.06 (m, 1H), 7.02-6.98 (m, 1H), 4.43-4.36 (m, 4H), 3.90 (s, 3H), 2.53 (s, 3H); MS m/z 507 (M+1).

Step 2: Lithium 2-[(2,4-dichlorobenzoyl)amino]-5-(2-{[2-(methylsulfanyl)-3-pyridinyl]oxy}ethoxy)benzoate

Use of methyl 2-[(2,4-dichlorobenzoyl)amino]-5-(2-t[2-(methylsulfanyl)-3-pyridinyl]oxy}ethoxy)benzoate afforded the title compound (98 mg, 100%) as a beige solid by the application of the general procedure B described above. ¹H NMR (DMSO) δ 14.86 (s, 1H), 8.50 (d, J=8.71 Hz 1H), 8.08 (d, J=4.48 Hz 1H), 7.76-7.49 (m, 4H), 7.35 (d, J=7.65 Hz 1H), 7.14-6.94 (m, 2H), 4.49-4.22 (m, 4H), 2.41 (s, 3H); MS m/z 491 (M−1).

### Example 11

Lithium 2-[(2,4-dichlorobenzoyl)amino]-5-[2-(3-pyridinyloxy)ethoxy]benzoate

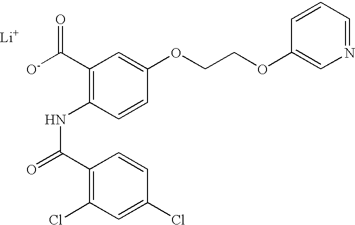

Step 1: Methyl 2-[(2,4-dichlorobenzoyl)amino]-5-[2-(3-pyridinyloxy)ethoxy]benzoate

Use of 2-(3-pyridinyloxy)ethanol (disclosed in WO 00/76984) afforded the title compound (50 mg, 22%), as a white solid, by the application of the general procedure A described above. 1H NMR (CDCl₃) δ 11.31 (s, 1H), 8.81 (d, J=9.24 Hz 1H), 8.43-8.22 (m, 2H), 7.65-7.58 (m, 2H), 7.48 (d, J=1.84 Hz 1H), 7.37-7.32 (mn, 1H), 7.29-7.20 (m, 3H), 4.38 (br s, 4H), 3.90 (s, 3H); MS m/z 461 (M+1).

Step 2: Lithium 2-[(2,4-dichlorobenzoyl)amino]-5-[2-(3-pyridinyloxy)ethoxy]benzoate

Use of methyl 2-[(2,4-dichlorobenzoyl)amino]-5-[2-(3-pyridinyloxy)ethoxy]benzoate afforded the title compound (35 mg, 96%) as a white solid by the application of the general procedure B described above. ¹H NMR (CD₃OD) δ 15.06 (s, 1H), 8.48 (d, J=8.97 Hz 1H), 8.35 (d, J=2.91 Hz 1H), 8.18 (dd, J=4.49, 1.32 Hz 1H), 7.72 (d, J=1.85 Hz 1H), 7.64-7.30 (m, 5H), 6.95 (dd, J=8.98, 3.17 Hz 1H), 4.43-4.25 (m, 4H).

### Example 12

Lithium 2-[(2,4-dichlorobenzoyl)amino]-5-[2-(2-quinoxalinyloxy)ethoxy]benzoate

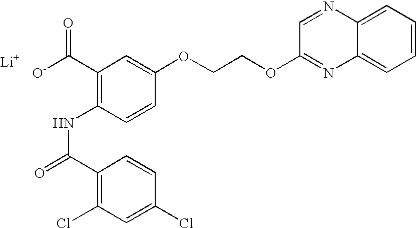

Step 1: Methyl 2-[(2,4-dichlorobenzoyl)amino]-5-[2-(2-quinoxalinyloxy)ethoxy]benzoate

Use of 2-(2-quinoxalinyloxy)ethanol (previously described in JP 06009622) afforded the title compound (65 mg, 25%), as a white solid, by the application of the general procedure A described above. ¹H NMR (CDCl₃) δ 11.29 (s, 1H), 8.74 (d, J=9.50 Hz 1H), 8.33 (s, 1H), 7.93-7.88 (m, 1H), 7.68-7.54 (m, 3H), 7.49-7.31 (m, 4H), 7.11-7.05 (m, 1H), 4.68 (t, J=5.67 Hz 2H), 4.38 (t, J=5.67 Hz 2H), 3.88 (s, 3H); MS m/z 512 (M+1).

Step 2: Lithium 2-[(2,4-dichlorobenzoyl)amino]-5-[2-(2-quinoxalinyloxy)ethoxy]benzoate

Use of methyl 2-[(2,4-dichlorobenzoyl)amino]-5-[2-(2-quinoxalinyloxy)ethoxy]benzoate afforded the title compound (46 mg, 80%) as a white solid by the application of the general procedure B described above. ¹H NMR (CD₃OD) δ 8.50 (d, J=8.97 Hz 1H), 8.23 (s, 11H), 7.91-7.37 (m, 8H), 6.97-6.87 (m, 11H), 4.78-4.70 (m, 2H), 4.44-4.37 (m, 2H); MS m/z 498 (M+1).

### Example 13

Lithium 2-[(2,4-dichlorobenzoyl)amino]-5-{2-[2-(methylsulfanyl)phenoxy]ethoxy}benzoate

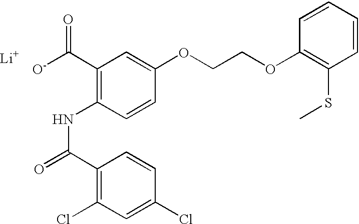

Step 1: Methyl 2-[(2,4-dichlorobenzoyl)amino]-5-{2-[2-(methylsulfanyl)phenoxy]ethoxy}benzoate

Use of 2-[2-(methylsulfanyl)phenoxy]ethanol (disclosed in WO 00/76984) afforded the title compound (6 mg, 2%), as a white solid, by the application of the general procedure A described above. ¹H NMR (CDCl₃) δ 11.31 (s, 1H), 8.79 (d, J=9.24 Hz 1H), 7.65-7.58 (m, 2H), 7.48 (d,J=1.85Hz 1H), 7.35 (dd, J=8.18 ,2.11 Hz 1H), 7.29-7.22 (m,11H), 7.20-7.10 (m, 2H), 7.03-6.95 (m, 1H), 6.93-6.88 (m, 1H), 4.40 (s, 4H), 3.90 (s, 3H), 2.42 (s, 3H); MS m/z 506 (M+1).

Step 2: Lithium 2-[(2,4-dichlorobenzoyl)amino]-5-{2-[2-(methylsulfanyl)phenoxy]ethoxy}benzoate

Use of methyl 2-[(2,4-dichlorobenzoyl)amino]-5-{2-[2-(methylsulfanyl)phenoxy]ethoxy}benzoate afforded the title compound (5 mg, 85%) as a white solid by the application of the general procedure B described above. MS m/z 490 (M−1).

### Example 14

Lithium 5-[2-(2-aminophenoxy)ethoxyl-2-[(2,4-dichlorobenzoyl)amino]benzoate

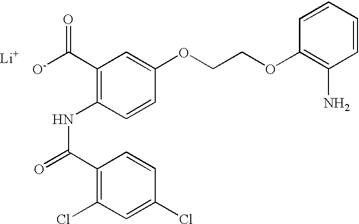

Step 1: Methyl 5-[2-(2-aminophenoxy)ethoxy]-2-[(2,4-dichlorobenzoyl)amino]benzoate

Use of 2-(2-aminophenoxy)ethanol (disclosed in EP 0881488 and EP 0881225) afforded the title compound (25 mg, 11 %), as a white solid, by the application of the general procedure A described above. ¹H NMR (CDCl₃) δ 11.31 (s, 1H), 8.80 (d, J=9.23 Hz 1H), 7.63-7.58 (m, 2H), 7.49 (d, J=1.85 Hz 1H), 7.35 (dd, J=8.18, 2.11 Hz 1H), 7.26-7.20 (m, 1H), 6.88-6.68 (m, 4H), 4.36 (s, 4H), 3.90 (s, 3H), 2.83 (br s, 2H); MS m/z 475 (M+1).

Step 2: Lithium 5-[2-(2-aminophenoxy)ethoxy]-2-[(2,4-dichlorobenzoyl)amino]benzoate

Use of methyl 5-[2-(2-aminophenoxy)ethoxy]-2-[(2,4-dichlorobenzoyl)amino]benzoate afforded the title compound (22 mg, 99%) as a beige solid by the application of the general procedure B described above. ¹H NMR (CD₃OD) δ 8.57 (d, J=8.97 Hz 1H), 7.74 (d, J=3.17 Hz 1H), 7.66-7.57 (m, 2H), 7.45 (dd, J=8.18, 2.11 Hz 1H), 7.09 (dd, J=8.98, 3.17 Hz 1H), 6.94-6.88 (m, 1H), 6.79-6.65 (m, 3H), 4.41-4.31 (m, 4H); MS m/z 459 (M−1).

### Example 15

Lithium 5-12-(4-benzoylphenoxy)ethoxyl-2-1(2,4-dichlorobenzoyl)amino]benzoate

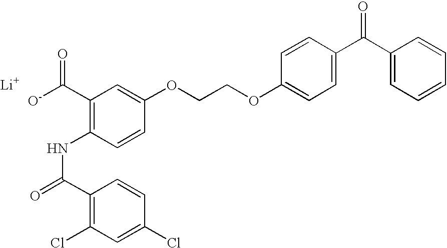

Step 1: Methyl 5-[2-(4-benzoylphenoxy)ethoxy]-2-[(2,4-dichlorobenzoyl)amino]benzoate

Use of [4-(2-hydroxyethoxy)phenyl](phenyl)methanone afforded the title compound (38 mg, 13%), as a white solid, by the application of the general procedure A described above. ¹H NMR (CDCl₃) δ 11.31 (s, 1H), 8.80 (d, J=9.24 Hz 1H), 7.83-7.78 (m, 2H), 7.65-7.55 (m, 3H), 7.52-7.32 (m, 7H), 7.27-7.16 (m, 2H), 4.38 (br s, 4H), 3.89 (s, 3H); MS m/z 564 (M+1).

Step 2: Lithium 5-[2-(4-benzoylphenoxy)ethoxy]-2-[(2,4-dichlorobenzoyl)amino]benzoate

Use of methyl 5-[2-(4-benzoylphenoxy)ethoxy]-2-[(2,4-dichlorobenzoyl)amino]benzoate afforded the title compound (30 mg, 80%) as a white solid by the application of the general procedure B described above. 1H NMR (DMSO) δ 15.07 (s, 1H), 8.48 (d, J=8.70 Hz 1H), 7.80-7.44 (m, IOH), 7.38-7.25 (m, 3H), 6.94 (dd, J=8.70, 2.90 Hz 1H), 4.42-4.23 (m, 4H); MS m/z 548 (M−1).

### Example 16

Lithium 2-[(2,4-dichlorobenzoyl)amino]-5-[2-(2,3,6-trifluorophenoxy)ethoxy]benzoate

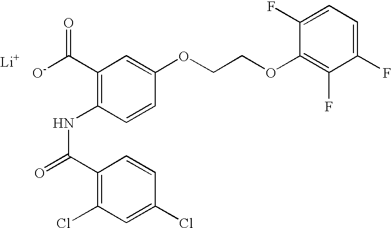

Step 1: Methyl 2-[(2,4-dichlorobenzoyl)amino]-5-[2-(2,3,6-trifluorophenoxy)ethoxy]benzoate

Use of 2-(2,3,6-trifluorophenoxy)ethanol afforded the title compound (67 mg, 26%), as a white solid, by the application of the general procedure A described above. ¹H NMR (CDCl₃) δ 11.31 (s, 1H), 8.80 (d, J=9.24 Hz 1H), 7.63-7.58 (m, 2H), 7.48 (d, J=2.11 Hz 1H), 7.35 (dd, J=8.31, 1.98 Hz 1H), 7.21 (dd, J=9.24, 3.17 Hz 1H), 7.04-6.86 (m, 2H), 4.35 (s, 4H), 3.90 (s, 3H); MS m/z 514 (M+1).

Step 2: Lithium 2-[(2,4-dichlorobenzoyl)amino]-5-[2-(2,3,6-trifluorophenoxy)ethoxy]benzoate

Use of methyl 2-[(2,4-dichlorobenzoyl)amino]-5-[2-(2,3,6-trifluorophenoxy)ethoxy]benzoate afforded the title compound (55 mg, 83%) as a beige solid by the application of the general procedure B described above. 1H NMR (DMSO) δ 15.03 (s, 1H), 8.49 (d, J 8.97 Hz 1H), 7.73 (d, J=2.11 Hz 1H), 7.67-7.44 (m, 5H), 6.94 (dd, J=8.71, 3.17 Hz 1H), 4.44-4.23 (m, 4H); MS m/z 498 (M−1).

### Example 17

Lithium 5-12-([1,1′-biphenyl]-3-yloxy)ethoxyl-2-[(2,4-dichlorobenzoyl)amino]benzoate

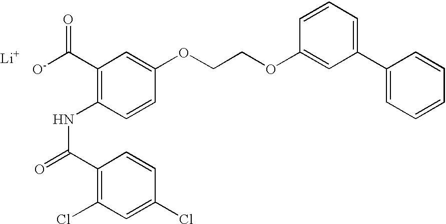

Step 1: Methyl 5-[2-([1,1′-biphenyl]-3-yloxy)ethoxy]-2-[(2,4-dichlorobenzoyl)amino]benzoate

Use of 2-(3-phenylphenoxy)ethanol (disclosed in WO 00/76984) afforded the title compound (55 mg, 21%), as a white solid, by the application of the general procedure A described above. 1H NMR (CDCl₃) δ 11.32 (s, 1H), 8.81 (d, J=9.24 Hz 1H), 7.66-7.55 (m, 4H), 7.49-7.31 (m, 6H), 7.28-7.17 (m, 3H), 6.97-6.91 (m, 1H), 4.39 (s, 4H), 3.89 (s, 3H); MS m/z 536 (M+l).

Step 2: Lithium 5-[2-([1,1′-biphenyl]-3-yloxy)ethoxy]-2-[(2,4-dichlorobenzoyl)amino]benzoate

Use of methyl 5-[2-([1,1′-biphenyl]-3-yloxy)ethoxy]-2-[(2,4-dichlorobenzoyl)amino]benzoate afforded the title compound (52 mg, 96%) as a white solid by the application of the general procedure B described above. ¹H NMR (DMSO) δ 15.04 (s, 1H), 8.50 (d, J=8.97 Hz 1H), 7.74-7.22 (m, 12H), 7.03-6.94 (m, 2H), 4.43-4.28 (m, 4H); MS m/z 520 (M−1).

### Example 18

Lithium 2-[(2,4-dichlorobenzoyl)amino]-5-[2-(phenylsulfanyl)ethoxy]benzoate

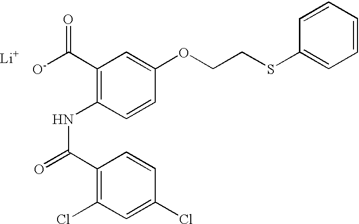

Step 1: Methyl 2-[(2,4-dichlorobenzoyl)amino]-5-[2-(phenylsulfanyl)ethoxy]benzoate

Use of 2-(phenylsulfanyl)ethanol afforded the title compound (29 mg, 12%), as a white solid, by the application of the general procedure A described above. 1H NMR (CDCl₃) δ 11.29 (s, 1H), 8.76 (d, J=9.24 Hz 1H), 7.61-7.57 (m, 1H), 7.52 (d, J=3.16 Hz 1H), 7.48 (d,J=1.85 Hz 1H), 7.44-7.38 (m, 2H), 7.36-7.27 (m, 3H), 7.25-7.18 (m, 1H), 7.12 (dd, J 9.24, 2.90 Hz 1H), 4.16 (t, J=6.86 Hz 2H), 3.88 (s, 3H), 3.29 (t, J=6.86 Hz 2H); MS m/z 476 (M+1).

Step 2: Lithium 2-[(2,4-dichlorobenzoyl)amino]-5-[2-(phenylsulfanyl)ethoxy]benzoate

Use of methyl 2-[(2,4-dichlorobenzoyl)amino]-5-[2-(phenylsulfanyl)ethoxy]benzoate afforded the title compound (25 mg, 88%) as a white solid by the application of the general procedure B described above. ¹H NMR (CD₃OD) δ 8.53 (d, J=8.97 Hz 1H), 7.65-7.56 (m, 3H), 7.47-7.40 (m, 3H), 7.34-7.16 (m, 3H), 6.96 (dd, J=8.97, 3.17 Hz 1H), 4.17 (t, J=6.60 Hz 2H), 3.32-3.28 (m, 2H); MS m/z 460 (M−1).

### Example 19

Lithium 2-[(2,4-dichlorobenzoyl)amino]-5-[2-(4-methyl-1,3-thiazol-5-yl)ethoxy]benzoate

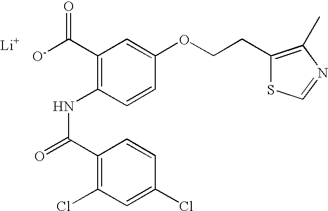

Step 1: Methyl 2-[(2,4-dichlorobenzoyl)amino]-5-(2-[(4-methyl-1,3-thiazol-5-yl)oxy]ethoxy}benzoate

Use of 2-(4-methyl-1,3-thiazol-5-yl)ethanol afforded the title compound (21 mg, 9%), as a white solid, by the application of the general procedure A described above. ¹H NMR (CDCl₃) δ 11.29 (s, 1H), 8.78 (d, J=9.23 Hz 1H), 8.60 (s, 1H), 7.62-7.56 (m, 1H), 7.53 (d, J =2.90 Hz 1H), 7.47 (d, J=1.85 Hz 1H), 7.34 (dd, J=8.19, 2.12 Hz 1H), 7.16 (dd, J=9.23, 3.17 Hz 1H), 4.15 (t, J=6.47 Hz 2H), 3.89 (s, 3H), 3.25 (t, J=6.47 Hz 2H), 2.45 (s, 3H); MS 465 m/z (M+1).

Step 2: Lithium 2-[(2,4-dichlorobenzoyl)amino]-5-(2-[(4-methyl-1,3-thiazo]-5-yl)oxy{ethoxy}benzoate

Use of methyl 2-[(2,4-dichlorobenzoyl)amino]-5-{2-[(4-methyl-1,3-thiazol-5-yl)oxy]ethoxy}benzoate afforded the title compound (18 mg, 90%) as a white solid by the application of the general procedure B described above. 1H NMR (DMSO) δ 15.09 (s, 1H), 8.82 (s, 1H), 8.46 (d, J=8.97 Hz 1H), 7.72 (d, J=1.85 Hz 1H), 7.63-7.50 (m, 3H), 6.89 (dd, J=8.97, 3.17 Hz 1H), 4.10 (t, J=6.07 Hz 2H), 3.20 (t, J=3.20 Hz 2H), 2.35 (s, 3H); MS m/z 449 (M−1).

### Example 20

Lithium 2-[(2,4-dichlorobenzoyl)amino]-5-(3-furylmethoxy)benzoate

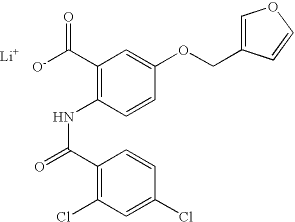

Step 1: Methyl 2-[(2,4-dichlorobenzoyl)amino]-5-(3-furylmethoxy)benzoate

Use of 3-furylalcohol afforded the title compound (37 mg, 18%), as a white solid, by the application of the general procedure A described above. ¹H NMR (CDCl₃) δ 11.30 (s, 1H), 8.78 (d, J=9.23 Hz 1H), 7.68-7.30 (m, 7H), 7.25-7.19 (m, 1H), 6.48 (s, 1H), 4.96 (s, 2H), 3.89 (s, 3H); MS 420 m/z (M+1).

Step 2: Lithium 2-[(2,4-dichlorobenzoyl)amino]-5-(3-furylmethoxy)benzoate

Use of methyl 2-[(2,4-dichlorobenzoyl)amino]-5-(3-furylmethoxy)benzoate afforded the title compound (31 mg, 86%) as a yellow solid by the application of the general procedure B described above. ¹H NMR (DMSO) δ 15.06 (s, 1H), 8.47 (d, J=8.97 Hz 1H), 7.78-7.49 (m, 7H), 6.96 (dd, J=8.97, 3.17 Hz 1H), 6.58-6.56 (m, 1H), 4.92 (s, 2H); MS m/z 404 (M−1).

### Example 21

Lithium 2-[(2,4-dichlorobenzoyl)amino]-5-[2-(2-thienyl)ethoxy]benzoate

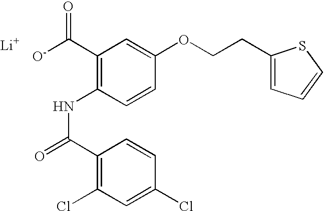

Step 1: Methyl 2-[(2,4-dichlorobenzoyl)amino]-5-[2-(2-thienyl)ethoxy]benzoate

Use of 2-(2-thienyl)ethanol afforded the title compound (38 mg, 17%), as an oil, by the application of the general procedure A described above. ¹H NMR (CDCl₃) δ 11.30 (s, 1H), 8.78 (d, J=9.24 Hz 1H), 7.62-7.55 (m, 2H), 7.47 (d, J=1.85 Hz 1H), 7.34 (dd, J=8.18, 2.11 Hz 1H), 7.21-7.16 (m, 2H), 6.98-6.91 (m, 2H), 4.21 (t, J=6.73 Hz 2H), 3.89 (s, 3H), 3.31 (t, J=6.60 Hz 2H); MS 450 m/z (M+1).

Step 2: Lithium 2-[(2,4-dichlorobenzoyl)amino]-5-[2-(2-thienyl)ethoxy]benzoate

Use of methyl 2-[(2,4-dichlorobenzoyl)amino]-5-[2-(2-thienyl)ethoxy]benzoate afforded the title compound (32 mg, 86%) as a white solid by the application of the general procedure B described above. ¹H NMR (DMSO) δ 14.96 (s, 1H), 8.48 (d, J=8.71 Hz 1H), 7.72 (d, J=1.85 Hz 1H), 7.64-7.50 (m, 3H), 7.37-7.32 (m, 1H), 7.00-7.68 (m, 3H), 4.15 (t, J =6.33 Hz 2H), 3.23 (t, J=6.33 Hz 2H); MS m/z 434 (M−1).

### Example 22

Lithium 2-[(2,4-dichlorobenzoyl)amino]-5-[2-(2-methyl-5-nitro-1H-imidazol-1-yl)ethoxy]benzoate

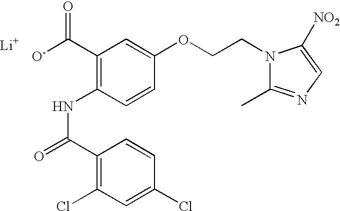

Step 1: Methyl 2-[(2,4-dichlorobenzoyl)amino]-5-[2-(2-methyl-5-nitro-]H-imidazol-1-yl)ethoxy/benzoate

Use of 1-(2-hydroxyethyl)-2-methyl-5-nitroimidazole afforded the title compound (53 mg, 21%), as a white solid, by the application of the general procedure A described above. ¹H NMR (CDCl₃) δ 11.28 (s, 1H), 8.75 (d, J=9.24 Hz 1H), 7.95 (s, 1H), 7.57 (d, J=8.18 Hz 1H), 7.45 (d, J=1.85 Hz 1H), 7.41 (d, J=3.17 Hz 1H), 7.32 (dd, J=8.18, 2.11 Hz 1H), 7.06 (dd, J=9.24, 3.17 Hz 1H), 4.71 (t, J=4.88 Hz 2H), 4.33 (t, J=4.75 Hz 2H), 3.88 (s, 3H), 2.61 (s, 3H); MS 493 m/z (M+1).

Step 2: Lithium 2-[(2,4-dichlorobenzoyl)amino]-5-[2-(2-methyl-5-nitro-1H-imidazol-1-yl)ethoxy]benzoate

Use of methyl 2-[(2,4-dichlorobenzoyl)amino]-5-[2-(2-methyl-5-nitro-1H-imidazol-1-yl)ethoxy]benzoate afforded the title compound (50 mg, 96%) as a brown-red solid by the application of the general procedure B described above. ¹H NMR (DMSO) δ 14.98 (s, 1 H), 8.45 (d, J=8.97 Hz 1H), 8.03 (s, 1H), 7.72 (d, J=1.84 Hz 1H), 7.64-7.47 (m, 3H), 6.86 (dd, J=8.97, 3.16 Hz 1H), 4.70 (t, J=5.01 Hz 2H), 4.29 (t, J=5.01 Hz 2H), 2.53 (s, 3H); MS m/z 477 (M−1).

### Example 23

Lithium 2-[(2,4-dichlorobenzoyl)amino]-5-(3-thienylmethoxy)benzoate

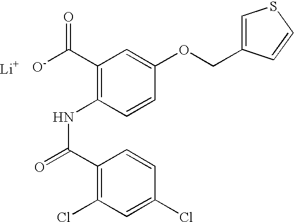

Step 1: Methyl 2-[(2,4-dichlorobenzoyl)amino]-5-(3-thienylmethoxy)benzoate

Use of thiophene-3-methanol afforded the title compound (30 mg, 14%), as a white solid, by the application of the general procedure A described above. ¹H NMR (CDCl₃) δ 11.30 (s, 1H), 8.78 (d, J=9.24 Hz 1H), 7.65-7.58 (m, 2H), 7.48 (d, J=2.11 Hz 1H), 7.37-7.32 (m, 3H), 7.23 (dd, J=9.24, 3.17 Hz 1H), 7.15 (dd, J=4.75, 1.59 Hz 1H), 5.09 (s, 2H), 3.89 (s, 3H); MS 436 m/z (M+1).

Step 2: Lithium 2-[(2,4-dichlorobenzoyl)amino]-5-(3-thienylmethoxy)benzoate

Use of methyl 2-[(2,4-dichlorobenzoyl)amino]-5-(3-thienylmethoxy)benzoate afforded the title compound (28 mg, 95%) as a white solid by the application of the general procedure B described above. ¹H NMR (DMSO) δ 15.03 (s, 1H), 8.47 (d, J=8.98 Hz 1H), 7.73 (d, J-=1.85 Hz 1H), 7.66-7.50 (m, 5H), 7.20-7.15 (m, 1H), 6.97 (dd, J=8.97, 3.17 Hz 1H), 5.05 (s, 2H); MS m/z 420 (M−1).

### Example 24

Lithium 2-[(2,4-dichlorobenzoyl)amino]-5-[2-(2-pyridinylsulfanyl)ethoxy]benzoate

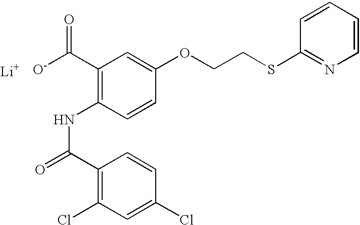

Step 1: Methyl 2-[(2,4-dichlorobenzoyl)amino]-5-[2-(2-pyridinylsulanyl)ethoxy]benzoate

Use of 2-(2-pyridylthio)ethanol afforded the title compound (1.7 mg, 1%), as an oil, by the application of the general procedure A described above. ¹H NMR (CDCl₃) δ 11.29 (s, 1H), 8.78 (d, J=8.98 Hz 1H), 8.46-8.43 (m, 1H), 7.63-7.57 (m, 2H), 7.51-7.45 (m, 2H), 7.37-7.32 (m, 1H), 7.29-7.19 (m, 2H), 7.03-6.98 (m, 1H), 4.27 (t, J=6.86 Hz 2H), 3.89 (s, 3H), 3.57 (t, J=6.86 Hz 2H); MS 477 mlz (M+1).

Step 2: Lithium 2-[(2,4-dichlorobenzoyl)amino]-5-[2-(2-pyridinylsulfanyl)ethoxy]benzoate

Use of methyl 2-[(2,4-dichlorobenzoyl)amino]-5-[2-(2-pyridinylsulfanyl)ethoxy]benzoate afforded the title compound (1.7 mg, 100%) as a white solid by the application of the general procedure B described above. MS m/z 469 (M+1).

### Example 25

Lithium 2-[(2,4-dichlorobenzoyl)amino]-5-[2-(5-methyl-2-phenyl-1,3-thiazol-4-yl)ethoxy]benzoate

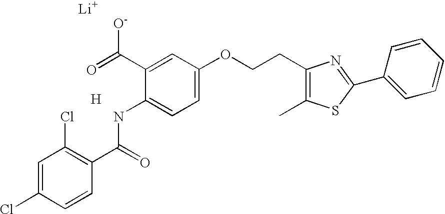

Step 3: Methyl 2-[(2,4-dichlorobenzoyl)amino]-5-[2-(5-methyl-2-phenyl-1,3-thiazol-4-yl)ethoxy]benzoate

Use of 2-(5-methyl-2-phenyl-1,3-thiazol-4-yl)ethanol afforded the title compound (170 mg, 63%) by the application of the general procedure A described above. ¹H NMR (DMSO) δ 10.72 (s, 1 H), 8.05-7.25 (aromatic signal, 11 H), 4.35 (triplet-like, 2 H), 3.76 (s, 3 H), 3.15 (triplet-like, 2 H), 2.44 (s, 3 H).

Step 2: Lithium 2-[(2,4-dichlorobenzoyl)amino]-5-[2-(5-methyl-2-phenyl-1,3-thiazol-4-yl)ethoxy]benzoate

Use of methyl 2-[(2,4-dichlorobenzoyl)amino]-5-[2-(5-methyl-2-phenyl-1,3-thiazol-4-yl)ethoxy]benzoate afforded the title compound (165 mg, 97%) as a solid by the application of the general procedure B described above. ¹H NMR (DMSO) δ 8.46 (d, J=8.9 Hz 1 H), 7.90-7.40 (aromatic signals, 9 H), 6.92 (dd, J=8.9, 3 Hz 1 H), 4.26 (t, J=7.2, 6.7 Hz 2 H), 3.12 (t, J=6.5, 6.7 Hz 2 H), 2.49 (s, 3 H); MS m/z (M+1) 527.

### Example 26

Lithium 2-[(2,4-dichlorobenzoyl)amino]-5-[2-(5-methyl-2-phenyl-1,3-oxazol-4-yl)ethoxy]benzoate

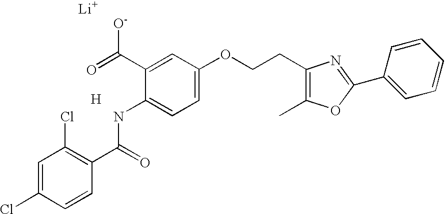

Step 1: Methyl 2-[(2,4-dichlorobenzoyl)amino]-5-[2-(5-methyl-2-phenyl-1,3-oxazol-4-yl)ethoxy]benzoate

Use of 2-(5-methyl-2-phenyl-1,3-oxazol-4-yl)ethanol afforded the title compound (145 mg, 55%) by the application of the general procedure A described above. ¹H NMR (DMSO) δ 8.47 (d, J=8.91 Hz 1H), 7.95-7.89 (m, 2H), 7.72 (d, J=1.98 Hz 1H), 7.64-7.45 (m, 6H), 6.96-6.89 (m, 1H), 4.18 (t, J=6.68 Hz 2H), 2.92 (t, J=6.68 Hz 2H), 2.36 (s, 3H); MS m/z (M+1) 511.

Step 2: Lithium 2-[(2,4-dichlorobenzoyl)amino]-5-[2-(5-methyl-2-phenyl-1,3-oxazol-4-yl)ethoxy]benzoate

Use of methyl 2-[(2,4-dichlorobenzoyl)amino]-5-[2-(5-methyl-2-phenyl-1,3-oxazol-4-yl)ethoxy]benzoate afforded the title compound (134 mg, 92%) by the application of the general procedure B described above. ¹H NMR (DMSO-d6) δ 8.50 (d, J=8.9 Hz 1 H), 7.99-7.36 (aromatic signals, 9 H), 6.90 (dd, J=8.9, 3.1 Hz 1 H), 4.20 (t, J=6.5 Hz 2 H), 2.90(t, J =6.5 Hz 2 H), 2.49 (s, 3 H); MS m/z (M+1) 511.

### Example 27

Lithium 2-[(2,4-dichlorobenzoyl)amino]-5-[2-(5-ethyl-2-pyridinyl)ethoxy]benzoate

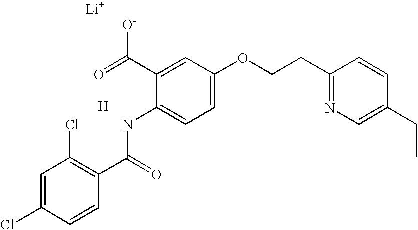

Step 1: Methyl 2-[(2,4-dichlorobenzoyl)amino]-5-[2-(5-ethyl-2-pyridinyl)ethoxy]benzoate

Use of 2-(5-ethyl-2-pyridinyl)ethanol afforded the title compound (90 mg, 38%) by the application of the general procedure A described above. ¹H NMR (CDCl₃) δ 11.28 (s, 1 H), 8.74 (d, J=10.5 Hz 1 H), 8.39 (s, 1 H), 7.70-7.05 (aromatic signals, 7 H), 4.35 (t, J=6.6 Hz 2 H), 3.87 (s, 3 H), 3.25 (t, J=6.6 Hz 2 H), 2.65 (dd, J=15.2, 7.5 Hz 2 H), 1.24 (t, J=7.5 Hz 3 H); MS m/z (M+1) 473.

Step 2: Lithium 2-[(2,4-dichlorobenzoyl)amino]-5-[2-(5-ethyl-2-pyridinyl)ethoy]benzoate

Use of methyl 2-[(2,4-dichlorobenzoyl)amino]-5-[2-(5-ethyl-2-pyridinyl)ethoxy]benzoate afforded the title compound (70 mg, 85%) by the application of the general procedure B described above. ¹H NMR (DMSO-d6) δ 14.98 (s, 1 H), 8.45 (d, J=8.1 Hz 1 H), 8.38 (s, 1 H), 7.85-6.80 (aromatic signals, 7 H), 4.28 (t, J=6.6, 6.6 Hz 2 H), 3.12 (t, J=6.6, 6.6Hz 2 H), 2.56 (dd, J=15.2, 7.5 Hz 2 H), 1.17 (t, J=7.5, 7.5 Hz 3 H); MS m/z (M+1) 459.

### Example 28

Lithium 2-[(2,4-dichlorobenzoyl)amino]-5-[2-(2-methoxyphenoxy)ethoxy]benzoate

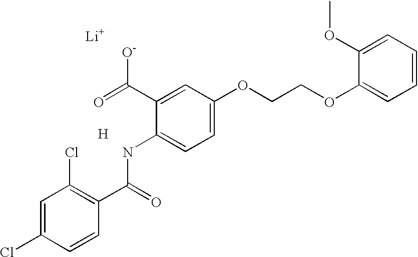

Step 1: Methyl 2-[(2,4-dichlorobenzoyl)amino]-5-[2-(2-methoxyphenoxy)ethoxy]benzoate

Use of 2-(2-methoxyphenoxy)ethanol (previously described in J. Chem. Soc. Dalton Trans. 1997, 449-462 and Org. Mass Spectrom. 1992, 995-999) afforded the title compound (167 mg, 68%) by the application of the general procedure A described above. ¹H NMR (CDCl₃) δ 11.31 (s, 1 H), 8.78 (d, J=8.1 Hz 1 H), 7.65-6.80 (aromatic signals, 9 H), 4.45-4.25 (overlapping signals, 4 H), 3.89 (s, 3 H), 3.85 (s, 3 H); MS m/z (M+1) 490.

Step 2: Lithium 2-[(2,4-dichlorobenzoyl)amino]-5-[2-(2-methoxyphenoxy)ethoxy]benzoate

Use of methyl 2-[(2,4-dichlorobenzoyl)amino]-5-[2-(2-methoxyphenoxy)ethoxy]benzoate afforded the title compound (163 mg, 98%) as a solid by the application of the general procedure B described above. ¹H NMR (DMSO-d6) δ 14.91 (s, 1 H), 8.48 (s, 1 H), 7.80-7.40 (aromatic signals, 4 H), 7.10-6.80 (overlapping signals, 5 H), 4.26 (s, 3 H), 3.85 (s, 4 H), 3.74 (s, 3 H); MS m/z (M−1) 475.

### Example 29

Lithium 2-[(2,4-dichlorobenzoyl)amino]-5-(4-pyridinylmethoxy)benzoate

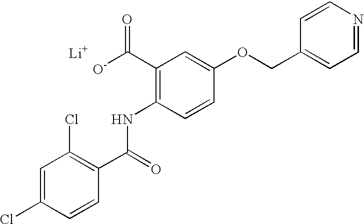

Step 1: Methyl 2-[(2,4-dichlorobenzoyl)amino]-5-(4-pyridinylmethoxy)benzoate

Use of 4-pyridinylmethanol afforded the title compound (38 mg, 16%) by the application of the general procedure A described above. ¹H NMR (CDCl₃) δ 11.31 (s, 1H), 8.81 (d, J=9.2 Hz 1H), 8.63 (br s, 2H), 7.63-7.58 (m, 2H), 7.48 (d, J=2.1 Hz 1H), 7.38-7.33 (m, 3H), 7.22-7.21 (m, 1H), 5.12 (s, 2H), 3.9 (s, 3H).

Step 2: Lithium 2-[(2,4-dichlorobenzoyl)amino]-5-(4-pyridinylmethoxy)benzoate

Use of methyl 2-[(2,4-dichlorobenzoyl)amino]-5-(4-pyridinylmethoxy)benzoate afforded the title compound (quantitative yield) as a white solid by the application of the general procedure B described above. ¹H NMR (DMSO-d6) δ 15.13 (s, 1H), 8.57-8.55 (m, 2H), 8.47 (d, J=9.0 Hz 1H), 7.72 (d, J=1.8 Hz 1H), 7.64-7.59 (m, 2H), 7.54-7.51 (m, 1H), 7.45-7.42 (m, 2H), 6.99 (dd, J=9.0, 3.2 Hz 1H), 5.16 (s, 2H); HRMS m/z calc. for C₂₀H₁₄Cl₂N₂O₄ (M)⁺ 416.0335, found 416.0331.

### Example 30

Lithium 2-[(2,4-dichlorobenzoyl)amino]-5-[3-(3-pyridinyl)propoxy]benzoate

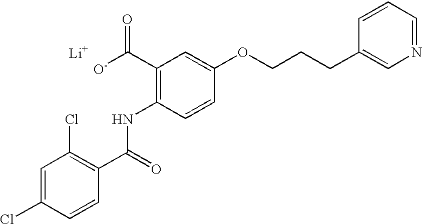

Step 1: Methyl 2-[(2,4-dichlorobenzoyl)amino]-5-[3-(3-pyridinyl)propoxy]benzoate

Use of 3-(3-pyridinyl)-1-propanol afforded the title compound (144 mg, 57%) by the application of the general procedure A described above. ¹H NMR (CDCl₃) δ 11.29 (s, 1H), 8.78 (d, J=9.2 Hz 1H), 8.49-8.45 (m, 2H), 7.61-7.47 (m, 4H), 7.36-7.32 (m, 1H), 7.24-7.14 (m, 2H), 3.99 (t, 2H), 3.89 (s, 3H), 2.86-2.81 (m, 2H), 2.17-2.07 (m, 2H).

Step 2: Lithium 2-[(2,4-dichlorobenzoyl)amino]-5-[3-(3-pyridinyl)propoxy]benzoate

Use of methyl 2-[(2,4-dichlorobenzoyl)amino]-5-[3-(3-pyridinyl)propoxy]benzoate afforded the title compound (quantitative yield) as a white solid by the application of the general procedure B described above. 1H NMR (DMSO-d6) δ 15.03 (s, 1H), 8.48-8.45 (m, 2H), 8.40 (dd, J=4.8, 1.6 Hz 1H), 7.72 (d, J=1.8 Hz 1H), 7.69-7.64 (m, 1H), 7.62-7.59 (m, 1H), 7.57-7.50 (m, 2H), 3.93 (t, J=6.3 Hz 2H), 3.61-3.56 (m, 2H), 2.79-2.74 (m, 2H), 2.07-1.97 (m, 2H), 1.77-1.72 (m, 2H);

HRMS m/z calc. for C₂₂H₁₈Cl₂N₂O₄ (M)⁺ 444.0644, found 444.0641

### Example 31

Lithium 2-[(2,4-dichlorobenzoyl)amino]-5-[2-(2-pyridinyl)ethoxy]benzoate

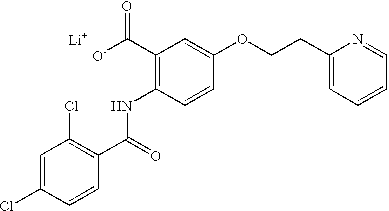

Step 1: Methyl 2-[(2,4-dichlorobenzoyl)amino]-5-[2-(2-pyridinyl)ethoxy]benzoate

Use of 2-(2-pyridinyl)ethanol afforded the title compound (47 mg, 19%) by the application of the general procedure A described above. ¹H NMR (CDCl₃) δ 11.28 (s, 1H), 8.75 (d, J=9.2 Hz 1H), 8.57-8.55 (m, 1H), 7.66-7.55 (m, 3H), 7.47 (d, J=1.8 Hz 1H), 7.35-7.32 (m, 1H), 7.28 (br s, 1H), 7.18-7.14 (m, 2H), 4.39 (t, J=6.4 Hz 2H), 3.88 (s, 3H), 3.27 (t, J=6.4 Hz 2H).

Step 2: Lithium 2-[(2,4-dichlorobenzoyl)amino]-5-[2-(2-pyridinyl)ethoxy]benzoate

Use of methyl 2-[(2,4-dichlorobenzoyl)amino]-5-[2-(2-pyridinyl)ethoxy]benzoate afforded the title compound (quantitative yield) as a white solid by the application of the general procedure B described above. ¹H NMR (DMSO-d6) δ 15.05 (s, 1H), 8.52-8.49 (m, 1H), 8.44 (d, J=9.0 Hz 1H), 7.77-7.69 (m, 2H), 7.62-7.50 (m, 3H), 7.37-7.35 (m, 1H), 7.26-7.21 (m, 1H), 6.88 (dd, J=9.0, 3.2 Hz 1H), 4.31 (t, J=6.6 Hz 2H), 3.61-3.56 (m, 1H), 3.17 (t, J=6.6 Hz 2H), 1.77-1.72 (m, 1H); HRMS m/z calc. for C₂₁H₁₆Cl₂N₂0₄ (M)⁺ 430.0487, found 430.0489

### Example 32

Lithium 2-[(2,4-dichlorobenzoyl)amino]-5-[2-(3-methoxyphenyl)ethoxy]benzoate

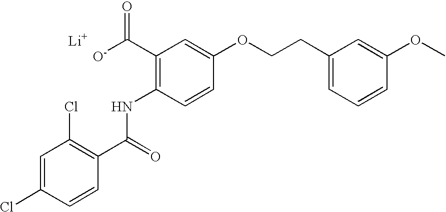

Step 1: Methyl 2-[(2,4-dichlorobenzoyl)amino]-5-[2-(3-methoxyphenyl)ethoxy]benzoate

Use of 2-(3-methoxyphenyl)ethanol afforded the title compound (48 mg, 18%) by the application of the general procedure A described above. 1H NMR (CDCl₃) δ 11.29 (s, 1H), 8.77 (d, J=9.2 Hz 1H), 7.61-7.58 (m, 1H), 7.53 (d, J=2.9 Hz 1H), 7.48 (d, J=1.8 Hz 1H), 7.36-7.32 (m, 1H), 7.28-7.14 (m, 2H), 6.89-6.78 (m, 3H), 4.18 (t, J=7.1 Hz 2H), 3.88 (s, 3H), 3.81 (s, 3H), 3.08 (t, J=7.1 Hz 2H).

Step 2: Lithium 2-[(2,4-dichlorobenzoyl)amino]-5-[2-(3-methoxyphenyl)ethoxy]benzoate

Use of methyl 2-[(2,4-dichlorobenzoyl)amino]-5-[2-(3-methoxyphenyl)ethoxy]benzoate afforded the title compound (quantitative yield) as a pink solid by the application of the general procedure B described above. ¹H NMR (DMSO-d6) δ 15.07 (s, 1H), 8.46 (d, J=9.0 Hz 1H), 7.62-7.50 (m, 3H), 7.24-7.18 (m, 1H), 6.91-6.87 (m, 3H), 6.80-6.76 (m, 1H), 4.14 (t, J=6.9 Hz 2H), 3.73 (s, 3H), 3.61-3.56 (m, 2H), 2.99 (t, J=6.6 Hz 2H), 1.77-1.72 (m, 2H).

### Example 33

Lithium 2-1[(2,4-dichlorobenzoyl)amino]-5-[(3-nitro-2-pyridinyl)oxy]benzoate

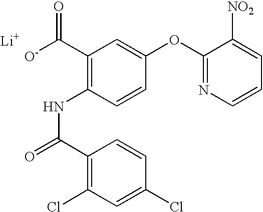

Step 1: Methyl 2-[(2,4-dichlorobenzoyl)amino]-5-[(6-nitro-2-pyridinyl)oxy]benzoate General procedure C

Methyl 2-[(2,4-dichlorobenzoyl)amino]-5-hydroxybenzoate (200 mg, 0.59 mmol) was added to a mixture of 2-chloro-3-nitropyridine (187 mg, 1.18 mmol) and potassium carbonate (244 mg, 1.8 mmol) in anhydrous dimethylformamide (6ml) and the suspension was stirred at 80° C. over night. After addition of chloroform, the reaction mixture was filtered and the filtrate concentrated in vacuo. The residue was treated with acetonitrile and the title compound (170 mg, 63%) could be filtered of as a white solid. ¹H NMR (CDCl₃) δ 11.55 (s, 1H), 8.99 (d, J=9.24 Hz 1H), 8.39 (dd, J=7.92, 1.85 Hz 1H), 8.33 (dd, J=4.75, 1.84 Hz 1H), 7.91 (d, J=2.64 Hz 1H), 7.61 (d, J=8.44 Hz 1H), 7.50 (d, J-=2.11 Hz 1H), 7.46 (dd, J=9.24, 2.90 Hz 1H), 7.37 (dd, J=8.44, 2.11 Hz 1H), 7.19 (dd, J=7.92,4.75 Hz 1H), 3.89 (s, 3H); MS 462 m/z (M+1).

Step 2: Lithium 2-[(2,4-dichlorobenzoyl)amino]-5-[(6-nitro-2-pyridinyl)oxy]benzoate

Use of methyl 2-[(2,4-dichlorobenzoyl)amino]-5-[(6-nitro-2-pyridinyl)oxy]benzoate afforded the title compound (88 mg, 94%) as a yellow solid by the application of the general procedure B described in Example 1. ¹H NMR (DMSO) δ 15.31 (s, 1H), 8.63-8.53 (m, 2H), 8.41 (dd, J=4.75, 1.84 Hz 1H), 7.94 (s, 1H), 7.75 (d, J=1.85 Hz 1H), 7.72 (d, J=2.91 Hz 1H), 7.66 (d, J=8.18 Hz 1H), 7.56 (dd, J=8.18, 1.85 Hz 1H), 7.36 (dd, J=7.91, 4.75 Hz 1H), 7.18 (dd, J=8.71, 2.90 Hz 1H); MS mlz 446 (M−1).

### Example 34

Lithium 2-[(2,4-dichlorobenzoyl)amino]-5-[(5-nitro-2-pyridinyl)oxy]benzoate

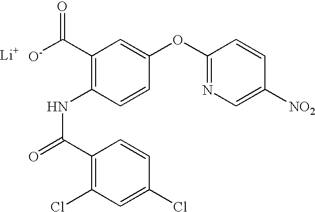

Step 1: Methyl 2-[(2,4-dichlorobenzoyl)amino]-5-[(5-nitro-2-pyridinyl)oxy]benzoate

Use of 2-chloro-5-nitropyridine afforded the title compound (89 mg, 33%), as a yellow solid, by the application of the general procedure C described above, with the exception that after filtration of the reaction mixture and evaporation, the residue was extracted between chloroform and brine. The organic phase was dried with magnesium sulphate and concentrated in vacuo. The crystalline residue was washed with diethyl ether to give the title compound. ¹H NMR (CDCl₃) δ 11.54 (s, 1H), 9.02-8.96 (m, 2H), 8.50 (dd, J=8.98, 2.91 Hz 1H), 7.88 (d, J=2.91 Hz 1H), 7.61 (d, J=8.18 Hz 1H), 7.50 (d, J=1.85 Hz 1H), 7.42 (dd, J=9.24,2.90 Hz 1H), 7.36 (dd, J=8.44,2.11 Hz 1H), 7.09 (d, J=8.71 Hz 1H), 3.89 (s, 3H); MS 462 m/z (M+1).

Step 2: Lithium 2-[(2,4-dichlorobenzoyl)amino]-5-[(5-nitro-2-pyridinyl)oxy]benzoate

Use of methyl 2-[(2,4-dichlorobenzoyl)amino]-5-[(5-nitro-2-pyridinyl)oxy]benzoate afforded the title compound (58 mg, 98%) as a yellow solid by the application of the general procedure B described above. ¹H NMR (CD₃OD) δ 9.00 (d, J=2.91 Hz 1H), 8.74 (d J=8.97 Hz 1H), 8.58 (dd, J=9.24, 2.90 Hz 1H), 7.88 (d, J=2.91 Hz 1H), 7.68 (d, J=8.44 Hz 1H), 7.60 (d, J=1.85 Hz 1H), 7.47 (dd, J=8.44, 2.11 Hz 1H), 7.28 (dd, J=8.97, 2.90 Hz 1H), 7.16 (d, J=9.24 Hz 1H); MS m/z 446 (M−1).

### Example 35

Lithium 2-[(2,4-dichlorobenzoyl)amino]-5-{1[5-(trifluoromethyl)-2-pyridinyl]oxy}benzoate

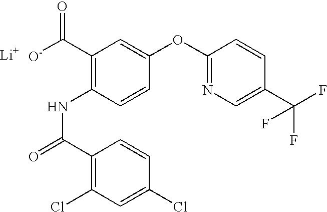

Step 1: Methyl 2-[(2,4-dichlorobenzoyl)amino]-5-{[5-(trifluoromethyl)-2-pyridinylloxy]benzoate

Use of 2-chloro-5-trifluoromethylpyridine afforded the title compound (44 mg, 15%), as a white solid, by the application of the general procedure C described above, with the exception that the crude product was purified by column chromatography on silica gel (eluting with chloroform) and re-crystallized from diethyl ether. ¹H NMR (CDCl₃) δ 11.53 (s, 1H), 8.97 (d, J=8.97 Hz 1H), 8.42 (s, 1H), 7.93 (dd, J=8.44, 2.11 Hz 1H), 7.87 (d, J=2.90 Hz 1H), 7.61 (d, J=8.18 Hz 1H), 7.50 (d, J=1.84 Hz 1H), 7.42 (dd, J=9.24, 2.90 Hz 1H), 7.37 (dd, J=8.45, 2.11 Hz 1H), 7.07 (d, J=8.71 Hz 1H), 3.89 (s, 3H); MS 485 m/z (M+1).

Step 2: Lithium 2-[(2,4-dichlorobenzoyl)amino]-5-([5-(trifluoromethyl)-2-pyridinylloxy]benzoate

Use of methyl 2-[(2,4-dichlorobenzoyl)amino]-5-{[5-(trifluoromethyl)-2-pyridinyl]oxy}benzoate afforded the title compound (41 mg, 95%) as a yellow solid by the application of the general procedure B described above. ¹H NMR (DMSO) δ 15.17 (s, 1H), 8.70-8.50 (m, 2H), 8.25-8.17 (m, 1H), 7.79-7.51 (m, 4H), 7.25-7.13 (m, 2H); MS m/z 469 (M−1).

### Example 36

Lithium 5-[(6-chloro-2-pyrazinyl)oxy]-2-[(2,4-dichlorobenzoyl)amino]benzoate

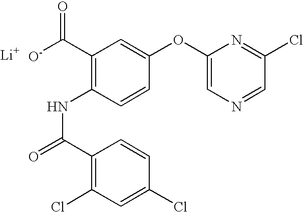

Step 1: Methyl 5-[(6-chloro-2-pyrazinyl)oxy]-2-[(2,4-dichlorobenzoyl)amino]benzoate

Use of 2-4-dichloropyrazine afforded the title compound (112.mg, 42%), as a white solid, by the application of the general procedure C described above. ¹H NMR (CDCl₃) δ 11.52 (s, 1H), 8.95 (d, J=9.23 Hz 1H), 8.32 (s, 1H), 8.29 (s, 1H), 7.86 (d, J=2.90 Hz 1H), 7.61 (d, J=8.44 Hz 1H), 7.49 (d, J=1.84 Hz 1H), 7.42 (dd, J=9.23, 2.90 Hz 1H), 7.36 (dd, J=8.19,2.11 Hz 1H), 3.89 (s, 3H); MS 452 m/z (M+1).

Step 2: Lithium 5-[(6-chloro-2-pyrazinyl)oxy]-2-[(2,4-dichlorobenzoyl)amino]benzoate

Use of methyl 5-[(6-chloro-2-pyrazinyl)oxy]-2-[(2,4-dichlorobenzoyl)amino]benzoate afforded the title compound (84 mg, 83%) as a white solid by the application of the general procedure B described above. 1H NMR (DMSO) δ 15.19 (s, 1H), 8.68-8.44 (m, 3H), 7.80-7.51 (m, 4H), 7.23 (dd, J=8.70, 2.91 Hz 1H); MS m/z 436 (M−1).

### Example 37

Lithium 2-[(2,4-dichlorobenzoyl)amino]-5-(2-pyrimidinyloxy)benzoate

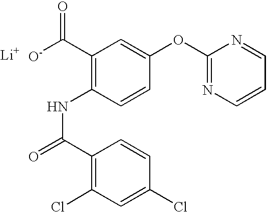

Step 1: Methyl 2-[(2,4-dichlorobenzoyl)amino]-5-[2-pyrimidinyloxy]benzoate

Use of 2-chloropyrimidine afforded the title compound (78 mg, 24%), as a white solid, by the application of the general procedure C described above. ¹H NMR (CDCl₃) δ 11.52 (s, 1H), 8.96 (d, J=9.23 Hz 1H), 8.56 (d, J=5.02 Hz 2H), 7.91 (d, J=2.90 Hz 1H), 7.60 (d, J=8.45 Hz 1H), 7.50-7.42 (m, 2H), 7.35 (dd, J=8.18, 1.85 Hz 1H), 7.06 (t, J=4.75 Hz 1H), 3.87 (s, 3H); MS 418 mlz (M+1).

Step 2: Lithium 2-[(2,4-dichlorobenzoyl)amino]-5-(2-pyrimidinyloxy)benzoate

Use of methyl 2-[(2,4-dichlorobenzoyl)amino]-5-(2-pyrimidinyloxy)benzoate afforded the title compound (64 mg, 90%) as a solid by the application of the general procedure B described above. ¹H NMR (DMSO) δ 15.11 (s, 1H), 8.70-8.55 (m, 3H), 7.80-7.52 (m, 4H), 7.29-7.12 (m, 2H); MS m/z 402 (M−1).

### Example 38

Lithium 2-[(2,4-dichlorobenzoyl)amino]-5-{[(2E)-3-phenyl-2-propenylloxy}benzoate

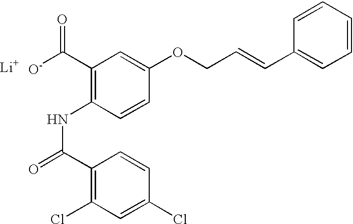

Step 1: Methyl 2-[(2,4-dichlorobenzoyl)amino]-5-{[(2E)-3-phenyl-2-propenyl]oxy}benzoate General procedure D

Cinnamyl bromide (131 μl, 0.88 mmol) was added to a stirred mixture of methyl 2-[(2,4-dichlorobenzoyl)amino]-5-hydroxybenzoate (300 mg, 0.88 mmol) and potassium carbonate (185 mg, 1.3 mmol) in DMF (10 ml). After heating at 65° C. for 4 hours the mixture was allowed to cool and then chloroform was added. Filtration and concentration of the filtrate in vacuo gave a residue which subsequently was purified by chromatography on silica gel eluting with CHCl₃ to give the title compound (196 mg, 50%) as a white solid. ¹H NMR (CHCl₃) δ 11.30 (s, 1H), 8.80 (d, J=9.15 Hz 1H), 7.64-7.20 (m, 11H), 6.75 (d, J=16.08 Hz 1H), 6.46-6.34 (m, 1H), 4.72 (dd, J=5.69, 1.49 Hz 2H), 3.90 (s, 3H); ); ¹³C NMR (CHCl₃) δ 168.3, 164.2, 154.1, 136.9, 136.3, 134.9, 134.8, 133.6, 132.3, 130.5, 130.4, 128.7, 128.1, 127.6, 126.7, 123.9, 122.3, 121.8, 116.7, 116.2, 100.0, 69.2, 52.7; MS m/z456(M+1).

Step 2: Lithium 2-[(2,4-dichlorobenzoyl)amino]-5-([(2E)-3-phenyl-2-propenyl]oxy}benzoate

Use of methyl 2-[(2,4-dichlorobenzoyl)amino]-5-{[(2E)-3-phenyl-2-propenyl]oxy}benzoate afforded the title compound (160 mg, 96%) as a solid by the application of the general procedure B described above. ¹H NMR (CD₃OD) δ 8.57 (d, J=8.90 Hz 1H), 7.76-7.18 (m, lOH), 7.08 (dd, J=8.90, 2.96 Hz 1H), 6.81-6.72 (m, 1H), 6.53-6.41 (m, 1H), 4.72 (dd, J=5.69, 1.24 Hz 2H); MS m/z 440 (M−1).

### Example 39

Lithium 2-1[(2,4-dichlorobenzoyl)amino]-5-1(3-methoxybenzyl)oxy]benzoate

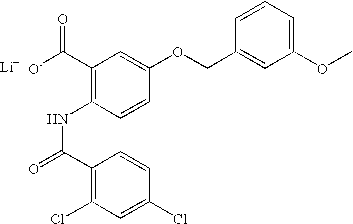

Step 1: Methyl 2-[(2,4-dichlorobenzoyl)amino]-5-[(3-methoxybenzyl)oxy]benzoate

Use of 3-methoxybenzyl bromide afforded the title compound (148 mg, 45%) as an oil by the application of the general procedure D described above. ¹H NMR (CD₃OD) δ 11.30 (s, 1H), 8.78 (d, J=9.15 Hz 1H), 7.66-7.57 (m, 2H), 7.47 (d, J=1.98 Hz 1H), 7.35-7.20 (m, 3H), 7.03-6.97 (m, 2H), 6.89-6.84 (m, 1H), 5.05 (s, 2H), 3.89 (s, 3H), 3.81 (s, 3H); ¹³C NMR (CHCl₃) δ 168.3, 164.1, 160.0, 154.2, 138.1, 136.9, 134.9, 134.7, 132.3, 130.5, 130.4, 129.8, 127.6, 122.2, 121.8, 119.7, 116.7, 116.3, 113.7, 113.1, 70.4, 55.3, 52.7; MS m/z 460 (M+1).

Step 2: Lithium 2-[(2,4-dichlorobenzoyl)amino]-5-[(3-methoxybenzyl)oxy]benzoate

Use of methyl 2-[(2,4-dichlorobenzoyl)amino]-5-[(3-methoxybenzyl)oxy]benzoate afforded the title compound (110 mg, 76%) as a white solid by the application of the general procedure B described above. ¹H NMR (CD₃0D) a 8.55 (d, J=8.91 Hz 1H), 7.75 (d, J=2.97 Hz 1H), 7.66-7.56 (m, 2H), 7.47-7.41 (m, 1H), 7.31-7.22 (m, 11H), 7.08 (dd, J=8.90, 2.96 Hz 1H), 7.04-6.98 (m, 2H), 6.89-6.82 (m, 1H), 5.07 (s, 2H), 3.79 (s, 3H); ¹³C NMR (CD₃OD) δ 172.6, 164.4, 160.0, 154.5, 138.9, 136.1, 133.4, 132.1, 129.9, 129.2, 127.4, 125.6, 120.6, 119.4, 117.7, 116.7, 113.1, 112.6, 100.0, 69.8, 54.3; MS m/z 444 (M−1).

### Example 40

Lithium 2-1[(2,4-dichlorobenzoyl)amino]-5-(3-thienyl)benzoate

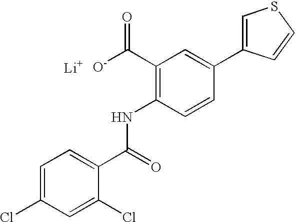

Step 1: Methyl 2-amino-5-iodobenzoate

To a solution of 2-amino-5-iodobenzoic acid (10 g, 38 mmol) in dry dioxane (280 ml) was added dropwise with stirring trichloromethylchloroformate (4.6 ml, 38 mmol). The vessel was allowed to stir for 1 hour at ambient temperature after which the solvent was removed at 50° C. by rotary evaporation. The residue was suspended in water (100 ml) and filtered and dried in a vacuum desiccator overnight to give the crude isatoic anhydride (13 g) IR; C═O stretch; 1795, cm⁻¹ and NH stretch; 3184-3088 cm⁻¹.

The crude isatoic anhydride (9.5 g, 27 mmol) was dissolved in dry methanol (670 ml) to which was added powdered anhydrous potassium carbonate (4.4 g, 31 mmol). The solution was allowed to stir at ambient overnight before being evaporated under reduced pressure. The residue was partitioned between ethyl acetate (200 ml) and water (200 ml) and the aqueous layer adjusted to pH 7.0 with concentrated hydrochloric acid before separating. The aqueous phase was extracted with two further portions of ethyl acetate (2×100 ml) and the combined fractions were dried (MgSO₄) and evaporated under reduced pressure to give a pale brown oil which solidified on standing (6.8g, 91%); ¹H NMR (CDCl₃) δ 8.12 (d, J=2.6Hz 1H), 7.46 (dd, J=8.7, 2.4Hz 1H), 6.45 (d, J=8.8Hz 1H), 3.86 (s, 3H).

Step 2: Methyl 2-[(2,4-dichlorobenzoyl)amino]-5-iodobenzoate General procedure E

To a stirred solution of methyl 2-amino-5-iodobenzoate (1.5 g, 5.4 mmol) in dry THF (30ml) cooled by an ice-water bath and under an atmosphere of nitrogen diusopropylethylamine (1.2 ml, 9.2 mmol) was added. A solution of 2,4-dichlorobenzoylchloride (0.84 ml, 6.0 mmol in 5 ml of THF) was added dropwise over ten minutes and the vessel was allowed to warm to ambient temperature slowly overnight after which the flask contents were filtered, the solid washed with ethyl acetate (20 ml) and the combined filtrate evaporated under reduced pressure. The residue was taken up in dichloromethane (50 ml) washed with an equal volume of IM hydrochloric acid, water and brine. The organic phase was then dried (MgSO₄) and evaporated under reduced pressure to give an off-white solid (2.25 g) which was re-crystallized from 1:1 hexane: dichloromethane to give colorless needles (1.88 g, 77%). ¹H NMR (CDCl₃) δ 11.49 (br s, 1H), 8.66 (d, J=9.0 Hz 1H), 8.38 (d, J=2.2Hz 1H), 7.89 (dd, J=8.8, 2.2 Hz 1H), 7.61 (d, J=8.3 Hz 1H), 7.50 (d, J=2.0 Hz 1H), 7.37 (dd, J=8.3, 2.0 Hz 1H), 3.92 (s, 3H); MS m/z (M−1) 448.

Step 3: Methyl 2-[(2,4-dichlorobenzoyl)amino]-5-(3-thienyl)benzoate General procedure F

To a stirred mixture of methyl 2-[(2,4-dichlorobenzoyl)amino]-5-iodobenzoate (100 mg, 0.22 mmol), tetrakis(triphenylphosphine)palladium(O) (13.5 mg, 5 mol%) and 3-thiopheneboronic acid (36 mg, 0.28 mmol) in degassed 1,2-dimethoxyethane (1.7 ml) was added sodium carbonate solution (0.25 ml, 2 N) before heating to reflux for 2.5 hours under an atmosphere of nitrogen. The solvent was removed under reduced pressure and the residue partitioned between dichloromethane (20 ml) and water (20 ml). The organic phase was separated, washed with brine (20 ml), dried (Na₂SO₄) and evaporated under reduced pressure to give the crude product. Purification by flash chromatography on a short column, eluting with a gradient from 100% hexane to 20% ethyl acetate in hexane, gave pale yellow needles (74 mg, 82%); ¹H NMR (CDCl₃) δ 11.56 (br s, 1H), 8.90 (d, J=8.8 Hz 1H), 8.30 (d, J=2.4 Hz 1H), 7.83 (dd, J=8.7, 2.3 Hz 1H), 7.63 (d, J=8.3 Hz 1H), 7.49 (br s, 2H), 7.41-7.34 (m, 3H) and 3.93 (s, 3H).

Step 4: Lithium 2-[(2,4-dichlorobenzoyl)amino]-5-(3-thienyl)benzoate General procedure G

To a stirred solution of methyl 2-[(2,4-dichlorobenzoyl)amino]-5-(3-thienyl)benzoate (37 mg, 0.091 mmol) in dioxane (1.1 ml) was added a solution of aqueous lithium hydroxide (2.46 mg in 1.1 ml). The reaction mixture was stirred overnight under an atmosphere of nitrogen and then freeze dried to give the pure lithium salt (25 mg, 70%) as a pale yellow solid; MS m/z 390 (M−1).

### Example 41

Lithium 2-1[(2,4-dichlorobenzoyl)amino]-5-(2,4-dichlorophenyl)benzoate

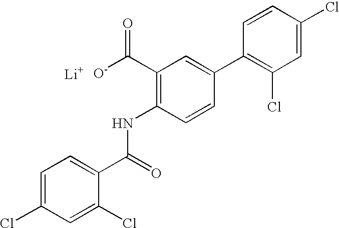

Step 1: Methyl 2-[(2,4-dichlorobenzoyl)amino]-5-(2,4-dichlorophenyl)benzoate

Use of 2,4-dichlorophenylboronic acid (42 mg, 0.22 mmol) afforded a 4:1 mixture of the title compound with characteristic NMR signals; ¹H NMR (CDCl₃) δ 11.38 (br s, 1H), 8.77 (d, J=9.0 Hz 1H), 8.24 (d, J=2.0 Hz 1H) 7.90 (dd, J=9.0, 2.0 Hz 1H), 3.81 (s, 3H) and the corresponding 2-aryl-1,3-benzoxazin-4-one with characteristic NMR signals; ¹H NMR (CDCl₃) δ 8.68 (d, J=9.0 Hz 1H), 8.36 (d, J=2.0 Hz 1H) by the application of the general procedure F described above.

Step 2: Lithium 2-[(2,4-dichlorobenzoyl)amino]-5-(2,4-dichlorophenyl)benzoate

To the crude mixture from step 1, dissolved in dioxane (3.3 ml) was added a solution of aqueous lithium hydroxide (7.2 mg in 3.3 ml). The reaction mixture was stirred overnight under an atmosphere of nitrogen and monitored by HPLC. A second aliquot of base (0.5 eq) was added followed by another 16 hours stirring at ambient temperature. The reaction mixture was then adjusted to pH 7.0 with concentrated hydrochloric acid, purified by prep HPLC and freeze dried to give the title compound (7.4 mg, 9%) as a colorless solid; MS m/z (M+1) 456.

### Example 42

2-1[(2,4-Dichlorobenzoyl)amino]-5-(4-ethylphenyl)benzoic acid

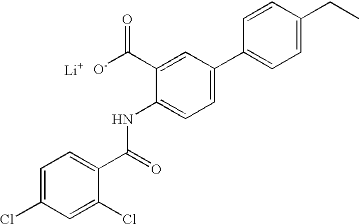

Step 1: 2-(2,4-dichlorophenyl)-6-(4-ethylphenyl)-J, 3-benzoxazin-4-one

Use of 4-ethyl phenylboronic acid (33 mg, 0.22 mmol) afforded the crude title compound by the application of the general procedure F described above. This was used in step 2 without further purification.

Step 2: 2-[(2,4-dichlorobenzoyl)amino]-5-(4-ethylphenyl)benzoic acid

Use of the crude 2-(2,4-dichlorophenyl)-6-(4-ethylphenyl)-1,3-benzoxazin-4-one (ca 0.18 mmol) afforded, after purification by HPLC, the title compound (23 mg, 32%) by the application of the general procedure G described above. MS m/z (M−1) 412.

### Example 43

2-[(2,4-Dichlorobenzoyl)amino]-5-(8-quinolinyl)benzoic acid

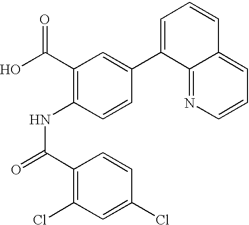

Step 1: 2-(2,4-Dichlorophenyl)-6-(quinolin-8-yl)-I, 3-benzoxazin-4-one

Use of quinoline-8-boronic acid (38 mg, 0.28 mmol) afforded the crude title compound by the application of the general procedure F described above. The crude product was used in the next step without further purification.

Step 2: 2-[(2,4-Dichlorobenzoyl)amino]-5-(8-quinolinyl)benzoic acid

Use of the crude 2-(2,4-dichlorophenyl)-6-(quinolin-8-yl)-1,3-benzoxazin-4-one afforded, after purification by HPLC, the title compound (12 mg, 15%) as an yellow solid by the application of the general procedure G described above. MS m/z (M+1) 437.

### Example 44

5-(1,3-Benzodioxol-5-yl)-2-[(2,4-dichlorobenzoyl)amino]benzoic acid

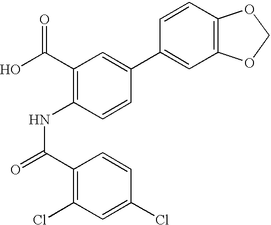

Step 1: Methyl 5-(1,3-benzodioxol-5-yl)-2-[(2,4-dichlorobenzoyl)amino]benzoate

Use of 1,3-benzodioxole-5-boronic acid (37 mg, 0.22 mmol) afforded the crude title compound by the application of the general procedure F described above. The crude product was used in the next step without further purification.

Step 2: 2-[(2,4-Dichlorobenzoyl)amino]-5-(8-quinolinyl)benzoic acid

Use of the crude methyl 5-(1,3-benzodioxol-5-yl)-2-[(2,4-dichlorobenzoyl)amino]benzoate afforded, after purification by HPLC, the title compound (16 mg, 21 %) as a solid by the application of the general procedure G described above. MS m/z (M−1) 428.

### Example 45

2-[(2,4-Dichlorobenzoyl)amino]-5-(2,4-dimethoxy-5-pyrimidinyl)benzoic acid

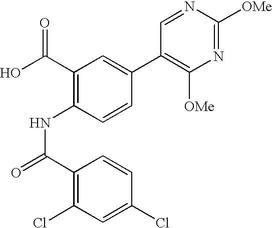

Step 1: Methyl 2-[(2,4-dichlorobenzoyl)amino]-5-(2,4-dimethoxy-5-pyrimidinyl)benzoate

Use of (2,4-dimethoxy)pyrimidine-5-boronic acid (41 mg, 0.22 mmol) afforded a crude mixture of title compound and the corresponding cyclized benzoxazine by the application of the general procedure F described above. The crude product was used in the next step without further purification.

Step 2: 2-[(2,4-Dichlorobenzoyl)amino]-5-(2,4-dimethoxy-5-pyrimidinyl)benzoic acid

Use of the mixture from step 1 afforded, after purification by HPLC, the title compound (17 mg, 21%) as an yellow solid by the application of the general procedure G described above. MS m/z (M+1) 448.

### Example 46

3′-(Acetylamino)-4-[(2,4-dichlorobenzoyl)aminol 11,1′-biphenyl]-3-carboxylic acid

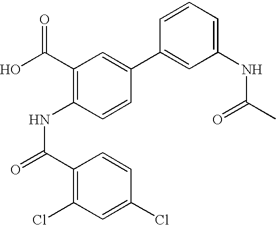

Step 1: Methyl 3′-(acetylamino)-4-[(2,4-dichlorobenzoyl)amino][1,1′-biphenyl]-3-carboxylate

Use of 3-(acetamido)phenylboronic acid (40 mg, 0.22 mmol) afforded a crude mixture of the title compound and the corresponding cyclized benzoxazine by the application of the general procedure F described above. The crude product was used in the next step without further purification.

Step 2: 3′-L(Acetylamino)-4-[(2,4-dichlorobenzoyl)amino][1,1′-biphenyl]-3-carboxylic acid

Use of the mixture from step 1 afforded, after purification by HPLC, the title compound (19 mg, 25%) as a solid by the application of the general procedure G described above. MS m/z (M−1) 441.

### Example 47

4-[(2,4-Dichlorobenzoyl)amino]-3′-(trifluoromethoxy)[1,1′-biphenyl]-3-carboxylic acid

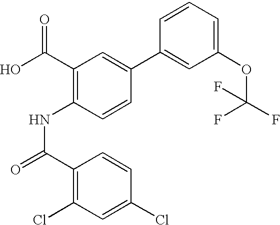

Step 1: Methyl 4-[(2,4-dichlorobenzoyl)amino]-3′-(trifluoromethoxy)[1,1′-biphenyl]-3-carboxylate

Use of 3-(trifluoromethoxy)phenylboronic acid (46 mg, 0.22 mmol) afforded a crude mixture of the title compound and the corresponding cyclized benzoxazine by the application of the general procedure F described above. The crude product was used in the next step without further purification.

Step 2: 4-[(2,4-Dichlorobenzoyl)amino]-3′-(trifluoromethoxy)[1,1′-biphenyl]-3-carboxylic acid

Use of the mixture from step 1 afforded, after purification by HPLC, the title compound (13 mg, 16%) as a solid by the application of the general procedure G described above. MS m/z (M−1) 468.

### Example 48

4-[(2,4-Dichlorobenzoyl)amino]-3′-ethoxy[1,1′-biphenyl]-3-carboxylic acid

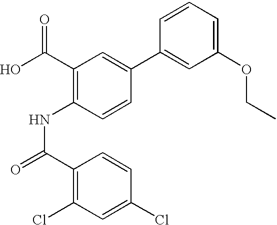

Step 1: Methyl 4-[(2,4-dichlorobenzoyl)amino]-3′-ethoxy[1,1′-biphenyl]-3-carboxylate

Use of 3-(ethoxy)phenylboronic acid (37 mg, 0.22 mmol) afforded a crude mixture of the title compound and the corresponding cyclized benzoxazine by the application of the general procedure F described above. The crude product was used in the next step without further purification.

Step 2: 4-[(2,4-Dichlorobenzoyl)amino]-3′-ethoxy[1,1′-biphenyl]-3-carboxylic acid

Use of the mixture from step 1 afforded, after purification by HPLC, the title compound (35 mg, 45%) as a solid by the application of the general procedure G described above. MS m/z (M−1) 428.

### Example 49

5-(1-Benzofuran-2-yI)-2-[(2,4-dichlorobenzoyl)amino]benzoic acid

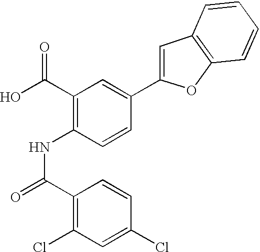

Step 1: Methyl 5-(1-benzofuran-2-yl)-2-[(2,4-dichlorobenzoyl)amino]benzoate

Use of benzofuran-2-boronic acid (23 mg, 0.14 mmol) afforded a crude mixture of the title compound and the corresponding cyclized benzoxazine by the application of the general procedure F described above. The crude product was used in the next step without further purification.

Step 2: 5-(1-Benzofuran-2-yl)-2-[(2,4-dichlorobenzoyl)amino]benzoic acid

Use of the mixture from step 1 afforded, after purification by HPLC, the title compound (4 mg, 9%) as an yellow solid by the application of the general procedure G described above. MS mlz (M−1) 424.

### Example 50

4-[(2,4-Dichlorobenzoyl)amino]-3′-(hydroxymethyl)[1,1′-biphenyll-3-carboxylic acid

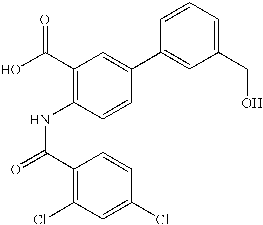

Step 1: Methyl 4-[(2,4-dichlorobenzoyl)amino]-3′-(hydroxymethyl)[1,1′-biphenyl]-3-carboxylate

Use of 3-(hydroxymethyl)phenylboronic acid (34 mg, 0.22 mmol) afforded a crude mixture of the title compound and the corresponding cyclized benzoxazine by the application of the general procedure F described above. The crude product was used in the next step without further purification.

Step 2: 4-[(2,4-Dichlorobenzoyl)amino]-3′-(hydroxymethyl)[1,1′-biphenyl]-3-carboxylic acid

Use of the mixture from step 1 afforded, after purification by HPLC, the title compound (20 mg, 27%) as a solid by the application of the general procedure G described above. MS m/z (M−1) 414.

### Example 51

4-[(2,4-Dichlorobenzoyl)amino]-3′-formyl[1,1′-biphenyl]-3-carboxylic acid

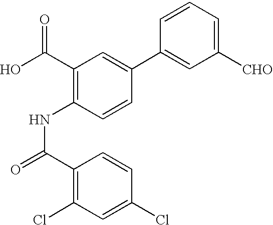

Step 1: Methyl 4-[(2,4-dichlorobenzoyl)amino]-3′-formyl[1,1′-biphenyl]-3-carboxylate

Use of 3-(forynyl)phenylboronic acid (33 mg, 0.22 mmol) afforded a crude mixture of the title compound and the corresponding cyclized benzoxazine by the application of the general procedure F described above. The crude product was used in the next step without further purification.

Step 2: 4-[(2,4-Dichlorobenzoyl)amino]-3′-formyl[1,1′-biphenyl]-3-carboxylic acid

Use of the mixture from step 1 afforded, after purification by HPLC, the title compound (6 mg, 8%) as a solid by the application of the general procedure G described above. MS m/z (M−1) 412.

### Example 52

2-1(2,4-Dichlorobenzoyl)amino]-5-(2-naphthyl)benzoic acid

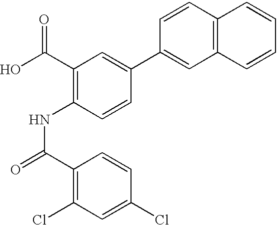

Step 1: 2-(2,4-Dichlorophenyl)-6-(naphth-2-yl)-1,3-benzoxazin-4-one

Use of naphthyl-2-boronic acid (38 mg, 0.22 mmol) afforded the crude title compound by the application of the general procedure F described above. The crude product was used in the next step without further purification.

Step 2: 2-[(2,4-Dichlorobenzoyl)amino]-5-(2-naphthyl)benzoic acid

Use of the crude 2-(2,4-dichlorophenyl)-6-(naphth-2-yl)-1,3-benzoxazin-4-one from step 1 afforded, after purification by HPLC, the title compound (8 mg, 10%) as a solid by the application of the general procedure G described above. MS m/z (M−1) 434.

### Example 53

4-[(2,4-Dichlorobenzoyl)amino]-3′-isopropyl-6′-methoxy[1,1′-biphenyll-3-carboxylic acid

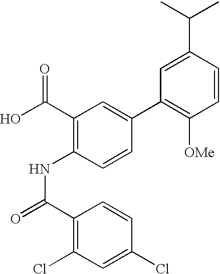

Step 1: Methyl 4-[(2,4-Dichlorobenzoyl)amino]-3′-isopropyl-6′methoxy[1,1′-biphenyl]-3-carboxylate

Use of 2-methoxy-5-isopropylphenylboronic acid (43 mg, 0.22 mmol) afforded a crude mixture of the title compound and the corresponding cyclized benzoxazine by the application of the general procedure F described above. The crude product was used in the next step without further purification.

Step 2: 4-[(2,4-Dichlorobenzoyl)amino]-3′-isopropyl-6′-methoxy[1,1′-biphenyl]-3-carboxylic acid

Use of the mixture from step 1 afforded, after purification by HPLC, the title compound (9 mg, 11 %) as an yellow solid by the application of the general procedure G described above. MS m/z (M−1) 456.

### Example 54

4-1(2,4-Dichlorobenzoyl)amino]-4′-fluoro[1,1′-biphenyl]-3-carboxylic acid

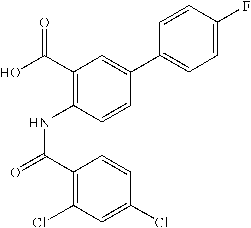

Step 1: Methyl 4-[(2,4-dichlorobenzoyl)amino]-4′-fluoro[1,1′-biphenyl]-3-carboxylate

Use of 4-fluorophenylboronic acid (31 mg, 0.22 mmol) afforded a crude mixture of the title compound and the corresponding cyclized benzoxazine by the application of the general procedure F described above. The crude product was used in the next step without further purification.

Step 2: 4-[(2,4-Dichlorobenzoyl)amino]-4′-fluoro[1,1′-biphenyl]-3-carboxylic acid

Use of the mixture from step 1 afforded, after purification by HPLC, the title compound (27 mg, 37%) as a solid by the application of the general procedure G described above. MS m/z (M−1) 402.

### Example 55

Lithium 2-[(2,4-dichlorobenzoyl)amino]-5-(2-furyl)benzoate

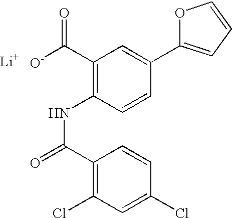

Step 1: Methyl 2-[(2,4-dichlorobenzoyl)amino]-5-(2-furyl)benzoate General procedure J

To a stirred mixture of methyl 2-[(2,4-dichlorobenzoyl)amino]-5-iodobenzoate (100 mg, 0.22 mmol), tetrakis(triphenylphosphine)palladium (13 mg, 5 mol%) and furan-2-boronic acid (31 mg, 0.27 mmol) in degassed 1,2-dimethoxyethane (1.7 ml) was added 2N sodium carbonate solution (0.25 ml) before heating for 5 hours at 80° C. under an atmosphere of nitrogen. The reaction mixture was washed with brine, dried (MgSO₄) and evaporated under reduced pressure to give the crude product. Purification by flash chromatography on a short silica column, eluting with a mixture of hexane and ethyl acetate (2: 1), gave a bright yellow solid (55 mg, 64%). ¹H NMR (CDCl₃) δ 11.56 (br s, 1H), 8.90 (d, J=8.8 Hz 1H), 8.37 (d, J=2.2 Hz 1H), 7.89 (dd, J=8.8, 2.2 Hz 1H), 7.63 (d, J=8.3 Hz 1H), 7.50 (s, 2H), 7.37 (dd, J=8.3, 1.9 Hz 1H), 6.68 (s, 1H), 6.51 (s, 1H), 3.95 (s, 3H).

Step 2: Lithium 2-[(2,4-dichlorobenzoyl)amino]-5-(2-furyl)benzoate

Use of methyl 2-[(2,4-dichlorobenzoyl)amino]-5-(2-furyl)benzoate afforded the title compound (53 mg, 100%) as a beige solid by the application of the general procedure G described above. MS m/z (M−1) 374.

### Example 56

Lithium 5-(1-benzothien-3-yl)-2-[(2,4-dichlorobenzoyl)aminol benzoate

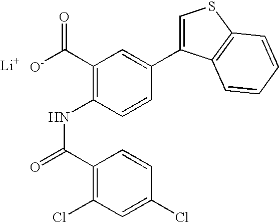

Step 1: Methyl 5-(1-benzothien-3-yl)-2-[(2,4-dichlorobenzoyl)amino]benzoate

Use of benzothiophene-3-boronic acid (49 mg, 0.27 mmol) afforded the title compound (54 mg, 74%) as a white solid by the application of the general procedure J described above. ¹H NMR (CDCl₃) δ 11.61 (br s,1H), 8.99 (d, J=8.8 Hz 1H), 8.31 (s, 1H), 7.96-7.83 (m, 3H), 7.65 (d, J=8.3 Hz 1H), 7.52 (s, 1H), 7.44-7.36 (m, 4H), 3.92 (s, 3H).

Step 2: Lithium 5-(]-benzothien-3-yl)-2-[(2,4-dichlorobenzoyl)amino]benzoate

Use of methyl 5-(1-benzothien-3-yl)-2-[(2,4-dichlorobenzoyl)amino]benzoate afforded the title compound (51 mg, 96%) as a white solid by the application of the general procedure G described above. MS m/z (M−1) 440.

### Example 57

Lithium 2-[(2,4-dichlorobenzoyl)amino]-5-(2-formyl-3-thienyl)benzoate

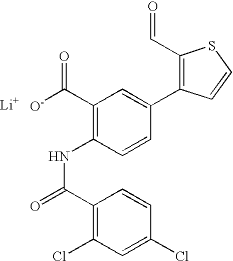

Step 1: Methyl 2-[(2,4-dichlorobenzoyl)amino]-5-(2-formyl-3-thienyl)benzoate

Use of 2-formylthiophene-3-boronic acid (43 mg, 0.27 mmol) afforded, after purification by HPLC, the title compound (31 mg, 32%) as a white solid by the application of the general procedure J described above. ¹H NMR (CDCl₃) δ 11.65 (br s, 1H), 9.89 (s, 1H), 9.01 (d, J=8.5 Hz 1H), 8.20 (d, J=1.9 Hz 1H), 7.79-7.72 (m, 2H), 7.65 (d, J=8.3 Hz 1H), 7.52 (d, J=1.9 Hz 1H), 7.39 (dd, J=8.3, 1.9 Hz 1H), 7.24 (s, 1H), 3.94 (s, 3H). Also isolated was the corresponding cyclized benzoxazine. 1H NMR (CD₃OD) δ 9.90 (s, 1H), 8.79 (d, J=8.5 Hz 1H), 8.28 (d, J=2.2 Hz 1H), 7.96 (dd, J=5.1, 1.2 Hz 1H), 7.69-7.59 (m, 3H), 7.47 (dd, J=8.3, 1.9 Hz 1H), 7.37 (d, J=5.1 Hz 1H).

Step 2: Lithium 2-[(2,4-dichlorobenzoyl)amino]-5-(2-formyl-3-thienyl)benzoate

Use of methyl 2-[(2,4-dichlorob enzoyl) amino]-5-(2-forrmyl-3-thienyl)benzoate afforded the title compound (31 mg, 84%) as an yellow solid by the application of the general procedure G described above. MS m/z (M−1) 418.

### Example 58

Lithium 5-(5-acetyl-2-thienyl)-2-[(2,4-dichlorobenzoyl)amino]benzoate

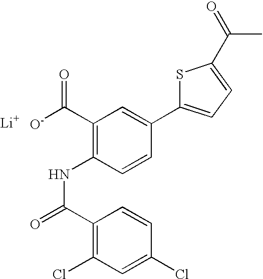

Step 1: Methyl 5-(5-acetyl-2-thienyl)-2-[(2,4-dichlorobenzoyl)amino]benzoate

Use of 5-acetylthiophene-2-boronic acid (47 mg, 0.27 mmol) afforded the title 20 compound (16 mg, 16%) as a white solid by the application of the general procedure J described above. 1H NMR (CDCl₃) δ 11.63 (br s, 1H), 8.95 (d, J=8.8 Hz 1H), 8.37 (d, J=2.4 Hz 1H), 7.88 (dd, J=8.8, 2.2 Hz 1H), 7.68-7.62 (m, 2H), 7.51 (d, J=1.9 Hz 1H), 7.40-7.34 (m, 2H), 3.96 (s, 3H), 2.58 (s, 3H).

Step 2: Lithium 5-(5-acetyl-2-thienyl)-2-[(2,4-dichlorobenzoyl)amino]benzoate

Use of methyl 5-(5-acetyl-2-thienyl)-2-[(2,4-dichlorobenzoyl)amino]benzoate afforded the title compound (13 mg, 100%) as an yellow solid by the application of the general procedure G described above. MS m/z (M−1) 432.

### Example 59

2-[(2,4-Dichlorobenzoyl)amino]-5-(1H-indol-5-yl)benzoic acid trifluoroacetate

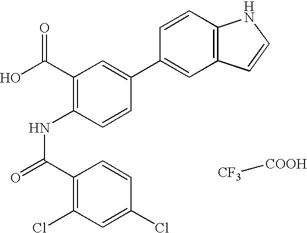

Step 1: Methyl 2-[(2,4-dichlorobenzoyl)amino]-5-(1H-indol-5-yl)benzoate

Use of 5-indolylboronic acid afforded the title compound (68 mg, 70%) as a beige solid by the application of the general procedure J described above. ¹H NMR (CDCl₃) δ 11.56 (br 5, 1H), 8.91 (d, J=8.5 Hz 1H), 8.36 (s, 2H), 7.91-7.86 (m, 2H), 7.61 (d, J=8.3 Hz 1H), 7.48 (s, 1H), 7.43 (s, 2H), 7.33 (d, J=8.3 Hz 1H), 6.61 (s, 1H), 3.93 (s, 3H).

Step 2: 2-[(2,4-Dich ornbenzoyl)amino]-5-(1H-indol-5-yl) benzoic acid tinfluoroacetate

Use of methyl 2-[(2,4-dichlorobenzoyl)amino]-5-( 1H-indol-5-yl)benzoate, 8 days reaction time and purification by HPLC afforded the title compound (31 mg, 37%) as a purple solid by the application of the general procedure G described above. ¹H NMR (CD₃COCD₃) δ 11.76 (br 5, 1H), 10.38 (br 5, 1H), 8.92 (d, J=8.5 Hz 1H), 8.46 (d, J=2.0 Hz 1H), 8.03 (dd, J=8.5, 2.0 Hz 1H), 7.92 (s, 1H), 7.80 (d, J=8.3 Hz 1H), 7.68 (s, 1H), 7.60-6.39 (m, 4H), 6.58 (s, 1H);

MS m/z (M−1) 423.

### Example 60

5-(3-Carboxyphenyl)-2-[(2,4-dichlorobenzoyl)amino]benzoic acid

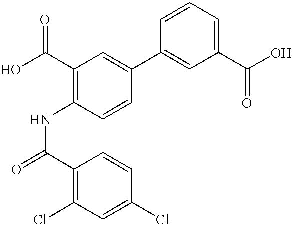

Step 1: Methyl 5-(3-carboxyphenyl)-2-[(2,4-dichlorobenzoyl)amino]benzoate

Use of 3-(carboxy)phenylboronic acid (47 mg, 0.28 mmol) afforded a crude mixture of the title compound and the corresponding cyclized benzoxazine by the application of the general procedure F described above. The crude product was used in the next step without further purification.

Step 2: 5-(3-Carboxyphenyl)-2-[(2,4-dichlorobenzoyl)amino]benzoic acid

Use of the mixture from step 1 afforded, after purification by HPLC, the title compound (24 mg, 72%) as a solid by the application of the general procedure G described above. MS m/z (M−1) 428.

### Example 61

Lithium 2′-(benzyloxy)-4-[(2,4-dichlorobenzoyl)amino]-[1,1′-biphenyl]-3-carboxylate

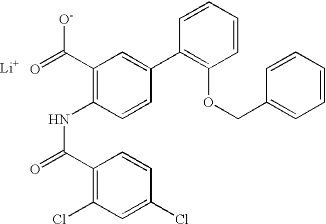

Step 1: Methyl 2′-(benzyloxy)-4-[(2,4-dichlorobenzoyl)amino][1,1′-biphenyl]-3-carboxylate

Use of 2-(benzyloxy)phenylboronic acid (63 mg, 0.28 mmol) afforded the title compound as a colorless solid (91 mg, 82%) by the application of the general procedure F described above.

¹H NMR (CDCl₃) δ 11.59 (br s, 1H), 8.89 (d, J=8.8 Hz 1H), 8.37 (d, J=2.2 Hz 2H), 7.85 (dd, J=8.8, 2.2 Hz 1H), 7.63 (d, J=8.3 Hz 1H), 7.50 (d, J=1.7 Hz 1H), 7.39-7.26 (m, 8H) 7.10-7.04 (m, 2H), 5.10 (s, 1H) and 3.87 (s, 3H); MS m/z (M+Na) 528.

Step 2: 2′-(benzyloxy)-4-[(2,4-dichlorobenzoyl)amino][J,1′-biphenyl]-3-benzoic acid

Use of methyl 2′-(benzyloxy)-4-[(2,4-dichlorobenzoyl)amino][1,1′-biphenyl]-3-carboxylate afforded the title compound (10 mg, 30%) as a colorless solid by the application of the general procedure G described above. MS m/z (M−1) 490.

### Example 62

Lithium 4-[(2,4-dichlorobenzoyl)amino][1,1′-biphenyl]-3-carboxylate

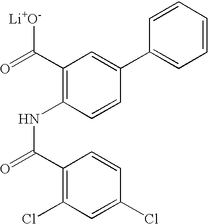

Step 1: Methyl 4-[(2,4-dichlorobenzoyl)amino][1,1′-biphenyll-3-carboxylate

Use of phenylboronic acid (34 mg, 0.28 mmol) afforded the title compound, after heating at reflux for 16 h, as a colorless solid (25 mg, 28%) by the application of the general procedure F described above. ¹H NMR (CDCl₃) δ 11.50 (br s, 1H), 8.87 (d, J=8.8 Hz 1H), 8.25 (d, J=2.4 Hz 1H), 7.79 (dd, J=8.8, 2.2 Hz 1H), 7.58-7.52 (m, 3H), 7.44-7.39 (m, 3H), 7.36-7.31 (m, 2H), 3.87 (s, 3H); MS m/z (M+1) 400.

Step 2: Lithium 4-[(2,4-dichlorobenzoyl)amino][1,1′-biphenyl]-3-carboxylate

Use of methyl 4-[(2,4-dichlorobenzoyl)amino][1,1′-biphenyl]-3-carboxylate) afforded the title compound (22 mg, 79%) as a colorless solid by the application of the general procedure G described above. MS m/z 384 (M−1).

### Example 63

Lithium 4-[(2,4-dichlorobenzoyl)amino]-3′-phenyl[1,1′-biphenyll-3-carboxylate

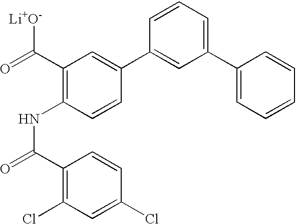

Step 1: Methyl 4-[(2,4-dichlorobenzoyl)amino]-3′-phenyl[1,1′-biphenyl]-3-carboxylate

Use of 3-(phenyl)phenylboronic acid (55 mg, 0.28 mmol) afforded the title compound as a colorless solid (8 mg, 74%) by the application of the general procedure F described above.

¹H NMR (CDCl₃) δ 11.59 (br s, 1H) 8.89 (d, J=8.8 Hz 1H), 8.37 (d, J=2.2 Hz 1H), 7.85 (dd, J=8.8, 2.2 Hz 1H), 7.63 (d, J=8.3 Hz 1H), 7.50 (d, J=1.7 Hz 1H), 7.39-7.26 (m, 8H), 7.10-7.04 (m, 2H), 5.10 (s, 1H), 3.87 (s, 3H); MS m/z (M+Na) 528.

Step 2: Lithium 4-[(2,4-dichlorobenzoyl)amino]-3′-phenyl[1,1′-biphenyl]-3-carboxylate

Use of methyl 4-[(2,4-dichlorobenzoyl)amino]-3′-phenyl[1,1′-biphenyl]-3-carboxylate afforded the title compound (38 mg, 99%) as a colorless solid by the application of the general procedure G described above. MS m/z (M−1) 460.

### Example 64

Lithium 4-[(2,4-dichlorobenzoyl)amino]-3′-(trifluoromethyl) [1,1′-biphenyl]-3-carboxylate

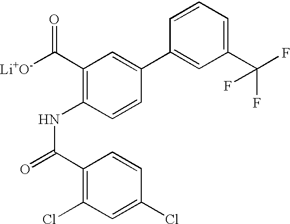

Step 1: Methyl 4-[(2,4-dichlorobenzoyl)amino]-3′-(trifluoromethyl)[1 ,1′-biphenyl]-3-carboxylate

Use of 3-(trifluoromethyl)phenylboronic acid (42 mg, 0.28 mmol) afforded the title compound (61 mg, 73%) as a solid by the application of the general procedure F described above. ¹H NMR (CDCl₃) δ 11.60 (br s, 1H), 9.0 (d, J=8.8 Hz 1H), 8.35 (d, J=2.2 Hz 1H), 7.90-7.41 (m, 8H), 3.99 (s, 3H).

Step 2: Lithium methyl 4-[(2,4-dichlorobenzoyl)amino]-3′-(trifluoromethyl)[1,1′-biphenyl]-3-carboxylate

Use of methyl 4-[(2,4-dichlorobenzoyl)amino]-3′-(trifluoromethyl)[1,1′-biphenyl]-3-carboxylate afforded the title compound (24 mg, 79%) as a solid by the application of the general procedure G described above. MS m/z (M−1) 452.

### Example 65

Lithium 5-(5-chloro-2-thienyl)-2-t(2,4-dichlorobenzoyl)amino]benzoate

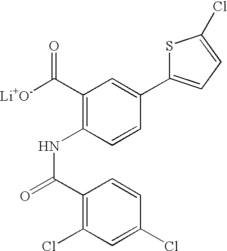

Step 1: Methyl 5-(5-chloro-2-thienyl)-2-[(2,4-dichlorobenzoyl)amino]benzoate

Use of 5-(chloro)thiophene-2-boronic acid (36 mg, 0.28 mmol) afforded the title compound (75 mg, 96%) as an yellow solid by the application of the general procedure F described above.

¹H NMR (CDCl₃) δ 11.56 (br s, 1H), 8.90 (d, J=8.8 Hz 1H), 8.19 (d, J=2.4 Hz 1H), 7.74 (dd, J=8.8,2.4 Hz 1H), 7.63 (d, J=8.3 Hz 1H), 7.50 (d, J=2.0 Hz 1H), 7.37 (dd, J-=8.3, 2.0 Hz 1H), 7.10 (d, J=3.9 Hz 1H), 6.91 (d, J=3.9 Hz 1H), 3.95 (s, 3H).

Step 2: Lithium 5-(5-chloro-2-thienyl)-2-[(2,4-dichlorobenzoyl)amino]benzoate

Use of methyl 5-(5-chloro-2-thienyl)-2-[(2,4-dichlorobenzoyl)amino]benzoate afforded the title compound (21 mg, 57%) as a brown solid by the application of the general procedure G described above. MS m/z (M−1) 426.

### Example 66

Lithium 4-[(2,4-dichlorobenzoyl)amino]-4′-phenoxy[1,1t-biphenyll-3-carboxylate

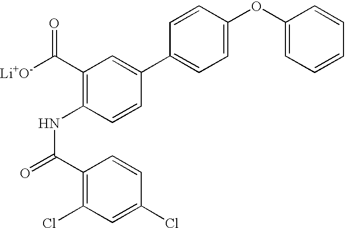

Step 1: Methyl 4-[(2,4-dichlorobenzoyl)amino]-4′-phenoxy[1,1′-biphenyl]-3-carboxylate

Use of 4-phenoxyphenylboronic acid (48 mg, 0.28 mmol) afforded the title compound (83 mg, 95%) as an yellow solid by the application of the general procedure F described above. ¹H NMR (CDCl₃) δ 11.56 (br s, 1H), 8.93 (d, J=8.8 Hz 1H), 8.28 (d, J=2.2 Hz 1H), 7.82 (dd, J=8.8, 2.4 Hz 1H), 7.72-7.32 (m 7H), 7.16-7.03 (m, 5H), 3.93 (s, 3H).

Step 2: Lithium 4-[(2,4-dichlorobenzoyl)amino]-4′-phenoxy[1,1′-biphenyl]-3-carboxylate

Use of methyl 4-[(2,4-dichlorobenzoyl)amino]-4′-phenoxy[1,1′-biphenyl]-3-carboxylate afforded the title compound (40 mg, 98%) as an yellow solid by the application of the general procedure G described above. MS m/z (M−1) 476.

### Example 67

Lithium 4-[(2,4-dichlorobenzoyl)amino]-2′,5′-dimethoxy[1,1′-biphenyl]-3-carboxylate

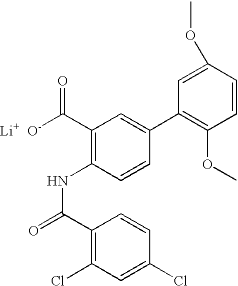

Step 1: Methyl 4-[(2,4-dichlorobenzoyl)amino]-2′,5′-dimethoxy[],]′-biphenyl]-3-carboxylate

Use of 2,5-(dimethoxy)phenylboronic acid (40 mg, 0.28 mmol) afforded the title compound (79 mg, 97%) as a solid by the application of the general procedure F described above. ¹H NMR (CDCl₃) δ 11.59 (br s, 1H), 8.90 (d, J=8.8 Hz 1H), 8.24 (d, J=2.2 Hz 1H), 7.82 (dd, J=8.8, 2.2 Hz 1H), 7.63 (d, J=8.3 Hz 1H), 7.50 (d, J=1.7 Hz 1H), 7.36 (dd, J=8.3, 2.0 Hz 1H), 7.00-6.83 (m, 3H), 3.91 (s, 3H) 3.82 (s, 3H), 3.76 (s, 3H).

Step 2: Lithium 4-[(2,4-dichlorobenzoyl)amino]-2,5′-dimethoxy[1,1′-biphenyl]-3-carboxylate

Use of methyl 4-[(2,4-dichlorobenzoyl)amino]-2′,5′-dimethoxy[1,1′-biphenyl]-3-carboxylate afforded the title compound (38 mg, 99%) as an yellow solid by the application of the general procedure G described above. MS m/z (M−1) 444.

### Example 68

3′-(Aminomethyl)-4-[(2,4-dichlorobenzoyl)aminol [1,1′-biphenyl]-3-carboxylic acid

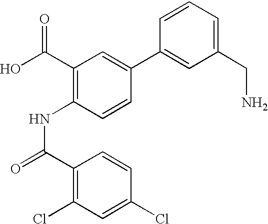

Step 1: 6-(3-Aminomethylphenyl)-2-(2,4-dichlorophenyl)-], 3-benzoxazin-4-one

Use of 3-aminomethylphenylboronic acid (38 mg, 0.14 mmol) afforded the title compound (23 mg, 50%) as a solid by the application of the general procedure F described above. ¹H NMR (CD₃COCD₃) δ 8.84 (d, J=8.3 Hz 1H), 8.44 (d, J=2.2 Hz 1H), 7.97 (dd, J=8.3, 1.7 Hz 1H), 7.90 (d, J=0.7 Hz 1H), 7.76 (d, J=8.1 Hz 1H), 7.74-7.65 (m, 1H), 7.65 (d, J=2.0 Hz 1H), 7.64-7.48 (m, 3H), 4.41 (s, 2H);

Step 2: 3′-(Aminomethyl)-4-[(2,4-dichlorobenzoyl)amino][J, 1′-biphenyl]-3-carboxylic acid

Use of 6-(3-aminomethylphenyl)-2-(2,4-dichlorophenyl)-1,3-benzoxazin-4-one afforded the title compound (4 mg, 18%) as a solid by the application of the general procedure G described above. MS m/z (M−1) 413.

### Example 69

Lithium 2-(2-naphthoylamino)-5-(3-thienyl)benzoate

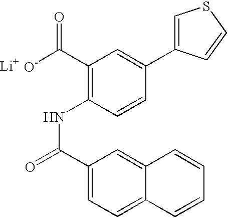

Step 1: Methyl 2-[(2-naphtoyl)amino]-5-iodobenzoate General procedure I

To a stirred solution of methyl 2-amino-5-iodobenzoate (500 mg, 1.8 mmol) in dry THF (10 ml) in a STEMBLOCK chiller at 0° C. under an atmosphere of nitrogen, dilsopropylethylamine (0.53 ml, 3.0 mmol) was added. A solution of 2-naphthoyl chloride (0.44 ml, 2.3 mmol) was added dropwise and the reaction mixture allowed to reach ambient temperature stirring overnight after which the flask contents were filtered, the solid washed with THF and the residue taken up in dichloromethane. The organic phase was washed with a solution of citric acid (5 %) and a solution of saturated sodium bicarbonate. The organic phase was then dried (MgSO₄) and concentrated in vacuo to give the title compound (0.4 g, 51%) as a white solid. ¹H NMR (CDCl₃) δ 12.12 (br s, 1H), 8.79 (d, J=9.0 Hz 1H), 8.56 (s, 1H), 8.41 (s, 1H), 8.10-7.87 (m, 5H), 7.64-7.54 (m, 2H), 4.00 (s, 3H). MS m/z (M+1) 432.

Step 2: Methyl 2-(2-naphthoylamino)-5-(3-thienyl)benzoate General B2

To a stirred mixture of methyl 2-[(2-naphtoyl)amino]-5-iodobenzoate (100 mg, 0.23 mmol), tetrakis(triphenylphosphine)palladium (13 mg, 5mol%) and thiophene-3-boronic acid (37 mg, 0.29 mmol) in degassed 1,2-dimethoxyethane (1.7 ml) and degassed toluene (0.5 ml) was added 2N sodium carbonate solution (0.26 ml) before heating for 32 hours at 80° C. under an atmosphere of nitrogen. The reaction mixture was washed with brine, dried (MgSO₄) and evaporated under reduced pressure to give the crude product. Purification by flash chromatography on a short column, eluting with a mixture of hexane and ethyl acetate (2:1), gave the title compound as a white solid (17 mg, 19%). ¹H NMR (CDCl₃) δ 12.20 (br s, 1H), 9.03 (d, J=8.8 Hz 1H), 8.60 (s, 1H), 8.34 (s, 1H), 8.13-7.84 (m, 5H), 7.60-7.57 (m, 2H), 7.50 (s, 1H), 7.43 (s, 2H), 4.02 (s, 3H). MS m/z (M+1) 388.

Step 3: Lithium 2-(2-naphthoylamino)-5-(3-thienyl)benzoate

Use of methyl 2-(2-naphthoylamino)-5-(3-thienyl)benzoate afforded the title compound (18 mg, 100%) as a yellow solid by the application of the general procedure G described above. MS m/z (M−1) 372.

### Example 70

Lithium 3′-(acetylamino)-4-(2-naphthoylamino)[1,1′-biphenyl]-3-carboxylate

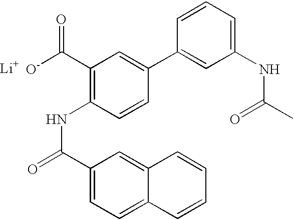

Step 1: Methyl 3′-(acetylamino)-4-(2-naphthoylamino)[1,1′-biphenyl]-3-carboxylate

Use of 3-acetamidophenylboronic acid (51 mg, 0.29 mmol) afforded the title compound (47 mg, 46%) as a beige solid by the application of the general procedure J described above. ¹H NMR (CDCl₃) δ 12.22 (br s, 1H), 9.03 (d, J=8.8 Hz 1H), 8.59 (s, 1H), 8.31 (s, 1H), 8.13-7.75 (m, 6H), 7.61-7.37 (m, 6H), 4.00 (s, 3H), 2.22 (s, 3H). MS m/z (M+1) 439.

Step 2: Lithium 3′-(acetylamino)-4-(2-naphthoylamino)[1,1′-biphenyl]-3-carboxylate

Use of methyl 3′-(acetylamino)-4-(2-naphthoylamino)[1,1′-biphenyl]-3-carboxylate afforded the title compound (39 mg, 100%) as a yellow solid by the application of the general procedure G described above. MS m/z (M−1) 423.

### Example 71

Lithium 3′-(hydroxymethyl)-4-(2-naphthoylamino)[1,1′-biphenyl]-3-carboxylate

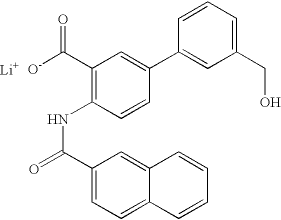

Step 1: Methyl 3′-(hydroxymethyl)-4-(2-naphthoylamino)[1, 1′-biphenyl]-3-carboxylate

Use of 3-(hydroxymethyl)phenylboronic acid (44 mg, 0.29 mmol) afforded the title compound (39 mg, 41 %) as a beige solid by the application of the general procedure J described above. ¹H NMR (DMSO-d6) δ 11.73 (br s, 1H), 8.68 (d, J=8.8 Hz 1H), 8.62 (s, 1H), 8.28 (s, 1H), 8.17-8.02 (m, 5H), 7.70-7.36 (m, 6H), 4.59 (s, 2H), 3.96 (s, 3H). MS m/z (M+1) 412.

Step 2: Lithium 3′-(hydroxymethyl)-4-(2-naphthoylamino)[1,′-biphenyl]-3-carboxylate

Use of methyl 3′-(hydroxymethyl)-4-(2-naphthoylamino)[1,1′-biphenyl]-3-carboxylate afforded the title compound (21 mg, 91%) as a yellow solid by the application of the general procedure G described above. MS m/z (M−1) 396.

### Example 72

Lithium 5-(3-thienyl)-2-{1[4-(trifluoromethyl)benzoyl]amino}benzoate

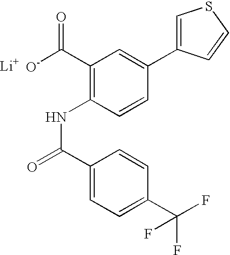

Step 1: Methyl 5-iodo-2-[(4-(trifluoromethyl)benzoyl)amino]benzoate

Methyl 2-amino-5-iodobenzoate (500 mg, 1.8 mmol) in dry THF (10 mL), diisopropylethylamine (0.53 ml, 3.0 mmol) and 4-(trifluoromethyl)benzoyl chloride (0.35 ml, 2.3 mmol) were reacted following the general procedure I. Purification by chromatography eluting with dichloromethane, followed by re-crystallization in a mixture of dichloromethane and hexane gave the title compound as a white solid (660 mg, 82%). ¹H NMR (CDCl₃) δ 12.10 (br s, 1H), 8.71 (d, J=9.0 Hz 1H), 8.41 (s, 1H), 8.14 (d, J=8.5 Hz 2H), 7.90 (d, J=9.0 Hz 1H), 7.80 (d, J=8.5 Hz 2H), 3.99 (s, 3H); MS m/z (M−1) 448.

Step 2: 6-(Thiophen3-yl)-2-[4-(trifluoromethyl)phenyl]-1,3-benzoxazine-4-one

Use of methyl 5-iodo-2-[(4-(trifluoromethyl)benzoyl)amino]benzoate (100 mg, 0.22 mmol), tetrakis(triphenylphosphine)palladium (13 mg, 5 mol%), thiophene-3-boronic acid (35 mg, 0.28 mmol) and 2N sodium carbonate (0.26 ml), in 1,2-dimethoxyethane (1.7 ml) at 80° C. for 32 hours afforded the title compound (66 mg, 80%) as a beige solid by the application of the general procedure J described above. ¹H NMR (DMSO-d6) δ 8.69 (d, J=8.5 Hz 1H), 8.36 (s, 1H), 8.23 (d, J=8.1 Hz 2H), 7.93 (d, J=8.3 Hz 2H), 7.78-7.51 (m, 4H). MS m/z (M−1, +H₂O) 390.

Step 3: Lithium 5-(3-thienyl)-2-{[4-(trifluoromethyl)benzoyl]amino}benzoate

Use of 6-(thiophen3-yl)-2-[4-(trifluoromethyl)phenyl]-1,3-benzoxazine-4-one afforded the title compound (22 mg, 63%) as a gray solid by the application of the general procedure G described above. MS m/z (M−1) 390.

### Example 73

Lithium 3′-(acetylamino)-4-{[4-(trifluoromethyl)benzoyl]amino}[1,1′-biphenyl]-3-carboxylate

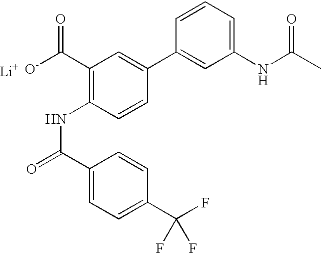

Step 1: 6-(3-Acetamideophenyl)-2-[4-(trifluoromethyl)phenyl]-1,3-benzoxazine-4-one

Use of methyl 5-iodo-2-[(4-(trifluoromethyl)benzoyl)amino]benzoate (100 mg, 0.22 mmol) and 3-acetamidophenylboronic acid (50 mg, 0.28 mmol) afforded the title compound (35 mg, 37%) as a solid by the application of the general procedure Jdescribed above. MS m/z (M−1, +H₂O) 441.

Step 2: Lithium 3′-(acetylamino)-4-([4-(trifluoromethyl)benzoyl]amino[1,1′-biphenyl]-3-carboxylate

Use of 6-(3-acetamideophenyl)-2-[4-(trifluoromethyl)phenyl]-1,3-benzoxazine-4-one afforded the title compound (22 mg, 63%) as a gray solid by the application of the general procedure G described above. MS m/z (M−1) 441.

### Example 74

Lithium 3′-(hydroxymethyl)-4-{[4-(trifluoromethyl)benzoyl]amino}[1,1′-biphenyl]-3-carboxylate

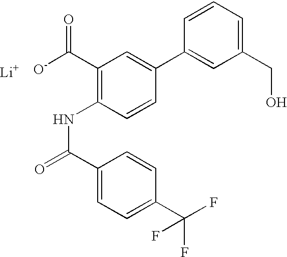

Step 1: 6-[3-Hydroxymethyl)phenyl]-2-[4-(trifluoromethyl)phenyl/]-1,3-benzoxazin-4-one

Use of methyl 5-iodo-2-[(4-(trifluoromethyl)benzoyl)amino]benzoate (100 mg, 0.22 mmol) and 3-(hydroxymethyl)phenylboronic acid (42 mg, 0.28 mmol) afforded the title compound (86 mg, 98%) as a white solid by the application of the general procedure J described above. MS m/z (M−1, +H₂O) 414.

Step 2: Lithium 3′-(hydroxymethyl)-4-([4-(trifluoromethyl)benzoyl]amino)[1,1′-biphenyl]-3-carboxylate

Use of 6-[3-hydroxymethyl)phenyl]-2-[4-(trifluoromethyl)phenyl]-1,3-benzoxazin-4-one afforded the title compound (50 mg, 73%) as an yellow solid by the application of the general procedure G described above. MS m/z (M−1) 414.

### Example 75

Lithium 2-{13,5-bis(trifluoromethyl)benzoyll amino}-5-(8-quinolinyl)benzoate

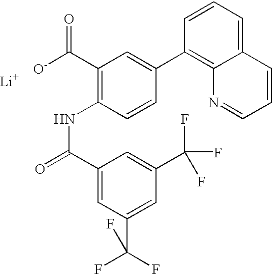

Step 1: Methyl 2-[(3,5-bis(trifluoromethyl)benzoyl)amino]-5-iodobenzoate

Use of 3,5-bis(trifluoromethyl)benzoyl chloride (0.42 ml, 2.3 mmol) and afforded the title compound (200 mg, 21%) as a white solid by the application of the general procedure I described above. ¹H NMR (CDCl₃) δ 12.27 (br s, 1H), 8.67 (d, J=9.0 Hz 1H), 8.48 (s, 2H), 8.43 (d, J=2.2 Hz 1H), 8.08 (s, 1H), 7.92 (dd, J=9.0, 2.2 Hz 1H), 4.00 (s, 3H). MS m/z (M−1) 516.

Step 2: Methyl 2-{[3,5-bis(trifluoromethyl)benzoyl]amino}-5-(8-quinolinyl)benzoate

Use of methyl 2-[(3,5-bis(trifluoromethyl)benzoyl)amino]-5-iodobenzoate (100 mg, 0.19 mmol) and 8-quinolineboronic acid (42 mg, 0.24 mmol) afforded the title compound (50 mg, 51 %) as a white solid by the application of the general procedure J described above. 1H NMR (CDCl₃) δ 12.45 (br s, 1H), 9.03-8.95 (m, 2H), 8.55 (s, 2H), 8.49 (d, J=2.2 Hz 1H), 8.23 (dd, J=8.3, 2.0 Hz 1H), 8.09 (s, 1H), 8.02 (dd, J=8.5, 2.2 Hz 1H), 7.84 (dd, J=8.1, 1.5 Hz 1H), 7.78-7.60 (m, 2H), 7.45 (dd, J=8.3, 4.1 Hz 1H), 3.99 (s, 3H).

Step 3: Lithium 2-{[3,5-bis(trifluoromethyl)benzoyl]amino}-5-(8-quinolinyl)benzoate

Use of methyl 2-{[3,5-bis(trifluoromethyl)benzoyl]amino}-5-(8-quinolinyl)benzoate afforded the title compound (47 mg, 100%) as solid by the application of the general procedure G described above. MS m/z (M−1) 503.

### Example 76

Lithium 4-{1[3,5-bis(trifluoromethyl)benzoyl]amino}-3′-formyl[1,1′-biphenyl]-3-carboxylate

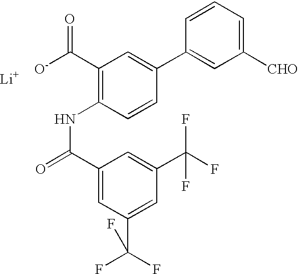

Step 1: Methyl 2-{[3,5-bis(trifluoromethyl)benzoyl]amino}-5-(3-formylphenyl)benzoate

Use of methyl 2-[(3,5-bis(trifluoromethyl)benzoyl)amino]-5-iodobenzoate (100 mg, 0.19 mmol) and 3-formylphenylboronic acid (36 mg, 0.24 mmol) afforded the title compound (6 mg, 6%) as a white solid by the application of the general procedure J described above. MS m/z (M−1) 494.

Step 2: Lithium 2-{[3,5-bis(trifluoromethyl)benzoyl]amino}-5-(3-formylphenyl)benzoate

Use of methyl 2-{[3,5-bis(trifluoromethyl)benzoyl]amino}-5-(3-fornylphenyl)benzoate afforded the title compound (7 mg, 100%) as a white solid by the application of the general procedure G described above. MS m/z (M−1) 480.

### Example 77

Lithium 2-[(4-methoxybenzoyl)amino]-5-(8-quinolinyl)benzoate

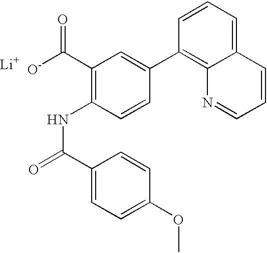

Step 1: Methyl 5-iodo-2-[(4-methoxybenzoyl)amino]benzoate

Use of 4-methoxybenzoyl chloride (0.58 ml, 3.4 mmol) and catalytic DMAP afforded the title compound (300 mg, 40%) as a beige solid by the application of the general procedure I described above. ¹H NMR (CDCl₃) δ 11.88 (br s, 1H), 8.73 (d, J=8.8 Hz 1H), 8.37 (d, J=2.2 Hz 11H), 8.00 (d, J=8.8 Hz 2H), 7.85 (dd, J=9.0, 2.2 Hz 1H), 7.01 (d, J=9.0 Hz 2H), 3.97 (s, 3H), 3.88 (s, 3H).

Step 2: Methyl 2-[(4-methoxybenzoyl)amino]-5-(8-quinolinyl)benzoate

Use of methyl 5-iodo-2-[(4-methoxybenzoyl)amino]benzoate (100 mg, 0.24 mmol) and 8-quinolineboronic acid (52 mg, 0.30 mmol) afforded the title compound (8 mg, 8%) as a beige solid by the application of the general procedure J described above. ¹H NMR (CDCl₃) δ 12.05 (br s, 1H), 9.07 (d, J=8.8 Hz 11H), 8.97-8.95 (m, 1H), 8.46 (s, 11H), 8.23 (d, J=8.3 Hz 1H), 8.07 (d, J=8.8 Hz 2H), 7.96 (d, J=8.6 Hz 1H), 7.85 (d, J=8.1 Hz 1H), 7.76 (d, J=7.2 Hz 1H), 7.65-7.59 (m, 1H), 7.46-7.41 (m, 1H), 7.04 (d, J=9.0 Hz 2H), 3.96 (s, 3H), 3.90 (s, 3H); MS m/z (M+1) 413.

Step 3: Lithium 2-[(4-methoxybenzoyl)amino]-5-(8-quinolinyl)benzoate

Use of methyl 2-[(4-methoxybenzoyl)amino]-5-(8-quinolinyl)benzoate afforded the title compound (6 mg, 100%) as a brown solid by the application of the general procedure G described above. MS m/z (M−1) 397.

### Example 78

Lithium 4-[(3,5-dichlorobenzoyl)amino][1,1′-biphenyll-3-carboxylate

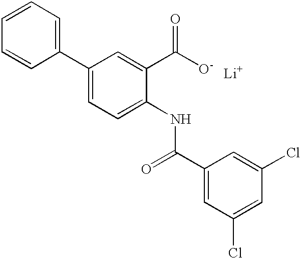

Step 1: Methyl 2-[(3,5-dichlorobenzoyl)amino]-5-bromobenzoate

Use of methyl-2-amino-5-bromobenzoate (1.0 g, 4.9 mmol) and 3,5-dichlorobenzoyl chloride (1.1 g, 5.4 mmol) afforded the title compound as a white solid (1.9 g, 86%) by the application of the general procedure E described above. ¹H NMR (CDCl₃) δ 12.01 (br s, 1H), 8.78 (d, J=9.0 Hz 1H), 8.23 (d, J=2.2 Hz 1H), 7.87 (s, 2H), 7.71 (dd, J=8.9, 2.3 Hz 1H), 7.56 (d, J=1.5 Hz 1H), 4.00 (s, 3H); MS m/z (M) 402.

Step 2: Methyl 4-[(3,5-dichlorobenzoyl)amino][1,1′-biphenyl]-3-carboxylate

Use of phenylboronic acid (38 mg, 0.31 mmol) afforded the title compound as a colorless solid (28 mg, 28%) by the application of the general procedure F described above. ¹H NMR (CDCl₃) δ 12.07 (br s, 1H), 8.89 (d, J=8.8 Hz 1H), 8.32 (d, J=2.4 Hz 1H), 7.90 (d, J=2.0 Hz 1H), 7.84 (dd, J=8.8, 2.2 Hz 1H), 7.60 (d, J=7.1 Hz 2H), 7.53 (s, 1H), 7.48-7.36 (m, 3H), 4.00 (s, 3H); MS m/z (M+1) 400.

Step 3: Lithium 4-[(3,5-dichlorobenzoyl)amino][1,]′-biphenyl]-3-carboxylate

Use of methyl 4-[(3,5-dichlorobenzoyl)amino][1,1′-biphenyl]-3-carboxylate afforded the title compound (16 mg, 87%) as a colorless solid by the application of the general procedure G described above. MS m/z (M−1) 384.

### Example 79

Lithium 2-[(4-cyanobenzoyl)amino]-5-(2-thienylmethoxy)benzoate

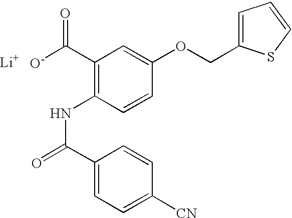

Step 1: Methyl 5-hydroxy-2-nitrobenzoate

To a stirred suspension of 5-hydroxy-2-nitrobenzoic acid (5.1 g, 28 mmol) in methanol (20 ml) was added sulfuric acid (95%, 8 ml) at room temperature. The solution was stirred at 90° C. for 1 hour after which it was allowed to reach room temperature and carefully poured into saturated sodium bicarbonate. Subsequent extraction with dichloromethane, drying of the organic phase using magnesium sulfate and concentration in vacuo gave the title compound (2.8 g, 52%) as a yellow solid. ¹H NMR (DMSO) δ 11.40 (br s, 1H), 8.04 (d, J=8.97 Hz 1H), 7.05-6.98 (m, 2H), 3.82 (s, 3H); MS m/z (M−1) 196.

Step 2: Methyl 2-nitro-5-(2-thienylmethoxy)benzoate

DEAD (4.0 ml, 25 mmol) was added to a solution of methyl 5-hydroxy-2-nitrobenzoate (3.3 g, 17 mmol), triphenylphosphine (6.6 g, 25 mmol) and thiophene-2-methanol (1.8 ml, 18 mmol) in anhydrous THF (40 ml) and the solution was stirred at room temperature over night. After being concentrated in vacuo the residue was purified by chromatography on silica gel, eluting with toluene, to give the title compound (700 mg, 14%). ¹H NMR (CDCl₃) ( 8.03 (d, J=8.98 Hz 1H), 7.37 (dd, J=5.01, 1.32 Hz 1H), 7.16-7.00 (m, 4H), 5.31 (s, 2H), 3.92 (s, 3H); MS m/z (M−1) 292.

Step 3: Methyl 2-amino-5-(2-thienylmethoxy)benzoate

To a vigorously stirred solution of methyl-2-nitro-5-(2-thienylmethoxy)benzoate (4.7 g, 16 mmol) in ethanol (180 ml) and THF (10 ml) was added Raney Nickel (in ethanol solution) and then immediately hydrazine hydrate (3.1 ml, 80 mmol). The mixture was stirred at room temperature for 1 hour, filtered through a pad of Celite (pretreated with water) and the filtrate was concentrated in vacuo to give an yellow oil. Purification by chromatography on silica gel, eluting with 10% methanol in dichloromethane, afforded the title compound (400 mg, 10%). MS m/z (M+1) 264.

Step 4: Methyl 2-[(4-cyanobenzoyl)amino/-5-(2-thienylmethoxy)benzoate General procedure H.

A mixture of 4-cyanobenzoic acid (56 mg, 0.38 mmol) and thionyl chloride (1.5 ml) was heated at 70° C. in a sealed glass vial for 2.5 hours and then concentrated in vacuo. To the residue was added polymer-supported N-methyl morpholine (380 mg, 0.76 mmol) and methyl 2-amino-5-(2-thienylmethoxy)benzoate (50 mg, 0.19 mmol) in anhydrous dichloromethane (4 ml) and the mixture was shaken at room temperature over night. Filtration of the reaction mixture and concentration of the filtrate in vacuo gave a solid which subsequently was re-crystallized in acetonitrile to afford the title compound as a white solid (35 mg, 47%). ¹H NMR (CDCl₃) δ 11.97 (s, 1H), 8.82 (d, J=9.24 Hz 1H), 8.14-8.08 (m, 2H), 7.83-7.77 (m, 2H), 7.68 (d, J=3.17 Hz 1H), 7.34 (dd, J=5.02, 1.06 Hz 1H), 7.25 (dd, J =9.23, 3.17 Hz 1H), 7.13-7.10 (m, 1H), 7.03-6.98 (m, 1H), 5.24 (s, 2H), 3.96 (s, 3H); MS m/z (M+1) 393.

Step 5: Lithium 2-[(4-cyanobenzoyl)amino]-5-(2-thienylmethoxy)benzoate

Use of 2-[(4-cyanobenzoyl)amino]-5-(2-thienylmethoxy)benzoate afforded the title compound (30 mg, 93%) as an yellow solid by the application of the general procedure B described above. ¹H NMR (DMSO) δ 8.58 (d, J=8.97 Hz 1H), 8.19-7.97 (m, 4H), 7.70 (d, J=2.90 Hz 1H), 7.54 (d, J=5.02 Hz 1H), 7.19 (d, J=2.90 Hz 1H), 7.06-6.97 (m, 2H), 5.26 (s, 2H); MS m/z (M−1) 377.

### Example 80

Lithium 2-[(2,4-difluorobenzoyl)amino]-5-(2-thienylmethoxy)benzoate

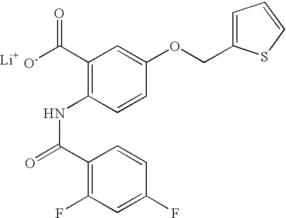

Step 1: Methyl 2-[(2,4-difluorobenzoyl)amino]-5-(2-thienylmethoxy)benzoate

Use of 2,4-difluorobenzoic acid (60 mg, 0.38 mmol) afforded the title compound (32 mg, 42%) as a white solid by the application of the general procedure H described above. ¹H NMR (CDCl₃) δ 11.60 (d, J=8.45 Hz 1H), 8.79 (d, J=9.23 Hz 1H), 8.16-8.06 (m, 1H), 7.67 (d, J=2.91 Hz 1H), 7.33 (dd, J=5.01, 1.32 Hz 1H), 7.23 (dd, J=9.50, 3.16 Hz 1H), 7.13-7.10 (m, 1H), 7.05-6.88 (m, 3H), 5.24 (s, 2H), 3.94 (s, 3H); ); MS m/z (M+1) 404.

Step 2: Lithium 2-[(2,4-difluorobenzoyl)amino]-5-(2-thienylmethoxy)benzoate

Use of methyl 2-[(2,4-difluorobenzoyl)amino]-5-(2-thienylmethoxy)benzoate afforded the title compound (10 mg, 57%) as a white solid by the application of the general procedure B described above. ¹H NMR (CD₃OD) δ 8.55 (d, J=9.23 Hz 1H), 8.00-7.87 (m, 1H), 7.74 (d, J=3.17 Hz 1H), 7.39 (dd, J=5.01, 1.05 Hz 1H), 7.18-6.96 (m, 5H), 5.27 (s, 2H); MS m/z (M−1) 388.

### Example 81

Lithium 2-[(2-chlorobenzoyl)amino]-5-1(3-nitro-2-pyridinyl)oxy]benzoate

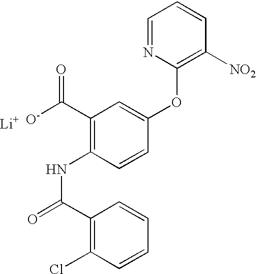

Step 1: Methyl 2-[(2-chlorobenzoyl)amino]-5-hydroxybenzoate

Use of 2-chlorobenzoic acid (580 mg, 2.4 mmol) afforded the title compound (460 mg, 63%) as a beige solid by the application of the general procedure H described above. ¹H NMR (CDCl₃) δ 11.25 (s, 1H), 8.72 (d, J=9.23 Hz 1H), 7.67-7.62 (m, 1H), 7.53 (d, J=2.90 Hz 1H), 7.48-7.31 (m, 3H), 7.12 (dd, J=9.23, 2.91 Hz 1H), 3.87 (s, 3H); MS m/z (M−1) 306.

Step 2: Methyl 2-[(2-chlorobenzoyl)amino]-5-[(3-nitro-2-pyridinyl)oxy]benzoate

Use of methyl 2-[(2-chlorobenzoyl)amino]-5-hydroxybenzoate (150 mg, 0.49 mmol) afforded the title compound (44 mg, 21%) as an yellow oil by the application of the general procedure C described above. ¹H NMR (CDCl₃) δ 11.49 (s, 1H), 9.02 (d, J=8.97 Hz 1H), 8.40-8.30 (m, 2H), 7.90 (d, J=2.90 Hz 1H), 7.67-7.62 (m, 1H), 7.49-7.35 (m, 4H), 7.21-7.15 (m, 1H), 3.88 (s, 3H); MS m/z (M+1) 428.

Step 3: Lithium 2-[(2-chlorobenzoyl)amino]-5-[(3-nitro-2-pyridinyl)oxy]benzoate

Use of methyl 2-[(2-chlorobenzoyl)amino]-5-[(3-nitro-2-pyridinyl)oxy]benzoate afforded the title compound (40 mg, 93%) as an yellow solid by the application of the general procedure B described above. ¹H NMR (DMSO) δ 15.03 (s, 1H), 8.68-8.53 (m, 2H), 8.44-8.38 (m, 1H), 7.74 (d, J=2.64 Hz 1H), 7.65-7.31 (m, 5H), 7.19 (dd, J=8.97, 2.90 Hz 1H); MS m/z (M−1) 412.

### Example 82

Lithium 2-[(2-chloro-5-nitrobenzoyl)amino]-5-(2-thienylmethoxy)benzoate

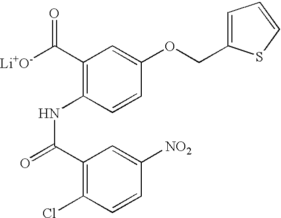

Step 1: Methyl 2-amino-5-(2-thienylmethoxy)benzoate hydrochloride

A solution of tert-butylazodicarboxylate (TMAD) (4.21g, 18.3 mmol) in dry THF (10 ml) was added dropwise to a solution of methyl-(2-amino-5-hydroxy)benzoate (2.03 g, 12.2 mmol; see Example 1), triphenylphosphine (6.40 g, 24.4 mmol) and thiophene-2-methanol (1.53 g, 13.4 mmol) in dry THF (90 ml) at 0° C., turning the solution from purple to green. After 15 minutes, the solution was warmed to ambient temperature and left to stir overnight. Concentration in vacuo followed by purification using flash chromatography on SiO₂ (gradient system 100% heptane-4%ethylacetate/heptane) gave a total of 1.68g of the free base. This was then dissolved in ether followed by addition of hydrochloric acid/ether, to give, after filtration and drying, (1.2 g, 33%) of the title compound as a white solid. ¹H NMR (CD₃OH): δ 7.75 (d, 1H), 7.40 (m, 3H), 7.20 (d, 1H), 7.00 (m, 1H), 5.35 (s, 2H), 3.95 (s, 3H); MS m/z (M) 263.

Step 2: Methyl 2-[(2-chloro-5-nitrobenzoyl)amino]-5-(2-thienylmethoxy)benzoate

Use of methyl 2-amino-5-(2-thienylmethoxy)benzoate hydrochloride (200 mg, 0.67 mmol) and 2-chloro-5-nitrobenzoic acid (135 mg, 0.67 mmol) afforded the title compound (70 mg, 23%) as an yellow solid by the application of the general procedure H described above. 1H NMR (CDCl₃) δ 11.47 (s, 1H), 8.79 (d, J=8.97 Hz 1H), 8.52 (d, J=2.64 Hz 1H), 8.25 (dd, J=8.71, 2.64 Hz 1H), 7.70-7.62 (m, 2H), 7.35 (dd, J=5.02, 1.06 Hz 1H), 7.30-7.24 (dd,J=9.24,3.17Hz 1H), 7.14-7.11 (m, 1H), 7.04-6.99 (m, 1H), 5.26 (s, 2H), 3.91 (s, 3H); MS m/z (M+1) 447.

Step 3: Lithium 2-[(2-chloro-5-nitrobenzoyl)amino]-5-(2-thienylmethoxy)benzoate

Use of methyl 2-[(2-chloro-5-nitrobenzoyl)amino]-5-(2-thienylmethoxy)benzoate afforded the title compound (30 mg, 88%) as an yellow solid by the application of the general procedure B described above. 1H NMR (DMSO) δ 15.40 (s, 1H), 8.48 (d, J=8.98 Hz 1H), 8.38 (d, J=2.64 Hz 1H), 8.31 (dd, J=8.70, 2.63 Hz 1H), 7.87 (d, J=8.71 Hz 1H), 7.66 (d, J=3.17 Hz 1H), 7.54 (dd, J=5.01, 1.05 Hz 1H), 7.21-7.17 (m, 1H), 7.06-6.98 (m, 2H), 5.26 (s, 2H); MS m/z (M−1) 431.

### Example 83

Lithium 2-[(2,4-dichlorobenzoyl)amino]-5-[2-(2-{[1-methyl-1H-imidazol-4-yl)sulfonyl]amino}ethoxy)ethoxy]benzoate

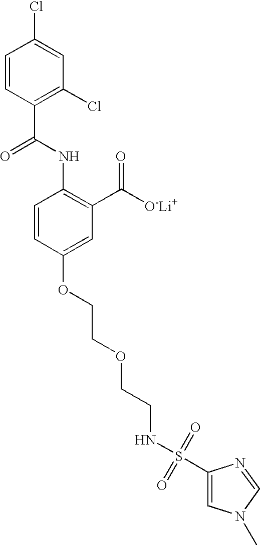

Step 1: 2-(2-N-Boc-aminoethoxy)ethanol

To a solution of 2-(2-aminoethoxy)ethanol (5.3 g, 50 mmol) and diisopropylethylamine (13 ml, 75 mmol) in DCM (10 ml) at 0° C. was added dropwise a is solution of di-tert-butyl dicarbonate (12 g, 55 mmol) in DCM (20 ml). The reaction was agitated for 4 hours, extracted with DCM and purified by flash chromatography (EtOAc/PE) (75/25) to give the title compound (5.9 g, 60%). ¹H NMR (CDCl₃) δ 3.65 (dd, J=8.6, 3.8 Hz 2H), 3.47 (m, 5H), 2.23 (dd, J=8.6, 3.8 Hz 2H), 1.35 (s, 9H); ¹³C NMR (CDCl₃) δ 56.1, 79.1, 72.1, 70.1, 61.3, 40.3, 28.2.

Step 2: Methyl 5-[2-(2-aminoethoxy)ethoxy]-2-[(2,4-dichlorobenzoyl)amino]benzoate trifluoroacetate

To methyl 2-[(2,4-dichlorobenzoyl)amino]-5-hydroxybenzoate (1.19 g, 3.50 mmol, from Example 1, Step 2) in dry THF (20 ml) were added 2-(2-N-Boc-aminoethoxy)-ethanol (0.9 g, 4.40 mmol) in dry THF and resin-bound Ph₃P. The suspension was cooled down to 0° C. before the addition of a dry THF (10 ml) solution of TMAD (0.9 g, 5.25 mmnol). The reaction was then agitated for 12 hours before complete conversion was assessed to have taken place by TLC analysis. After extraction in DCM and flash chromatography purification (Et Ac/PE) (40/60), the compound was agitated in (TFA/DCM) (50/50) (10 ml) for 4 hours. Conversion was complete as assessed by TLC analysis and the title product was isolated (1.7 g, 90%) as a solid. MS m/z (M+1) 427.

Step 3: Methyl 2-[(2,4-dichlorobenzoyl)amino]-5-[2-(2-{[(1-methyl-1H-imidazol-4-yl)sulfonyl]amino}ethoxy)ethoxy]benzoate

General procedure X

A solution of methyl 5-[2-(2-aminoethoxy)ethoxy]-2-[(2,4-dichlorobenzoyl)amino]benzoate trifluoroacetate (0.20 mmol), 1-methyl-1H-imidazole-4-sulfonyl chloride (0.21 mmol) and resin-bound DIPEA (0.70 mmol) in (acetonitrile/dichloroethane) (1/1) (3.5 ml) was agitated at room temperature for 20 hours and then at 75° C. for 5 hours. After filtration, the solvents were evaporated. The crude material was purified by preparative LC-MS yielding the title product (14 mg, 12%). MS m/z (M+1)571.

Step 4: Lithium 2-[(2,4-dichlorobenzoyl)amino]-5-[2-(2-([1-methyl-]H-imidazol-4-yl)sulfonyl{amino}ethoxy)ethoxy]benzoate

General procedure Y

To a solution of methyl 2-[(2,4-dichlorobenzoyl)amino]-5-[2-(2-{[(1-methyl-1H-imidazol-4-yl)sulfonyl]amino}ethoxy)ethoxy]benzoate in THF (2 ml) was added 2 eq of IM LiOH. The reaction was stirred for as long as need for the hydrolysis to be shown to be complete by TLC assessment. Evaporation of the solvents gave the title compound (12 mg, 89%); MS m/z (M−1) 555.

### Example 84

Lithium 2-[(2,4-dichlorobenzoyl)amino]-5-{2-[2-(2-pyridinylamino)ethoxy]ethoxy}benzoate

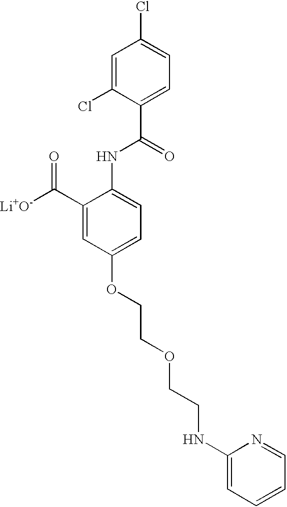

Step 1: Methyl 2-[(2,4-dichlorobenzoyl)amino]-5-(2-[2-(2-pyridinylamino)ethoxy]ethoxy) benzoate

A solution of methyl 5-[2-(2-aminoethoxy)ethoxy]-2-[(2,4-dichlorobenzoyl)amino]benzoate trifluoroacetate (0.42 mmol, prepared in Example 83, Step 2), 2-fluoropyridine (4.20 mmol) and TEA (6.30 mmol) in acetonitrile (10 ml) was heated at reflux for 4 days. The reaction was quenched with water, the products were extracted, and the solvent was concentrated. The crude material was purified by preparative LC-MS yielding the title compound (28 mg, 14%). ¹H NMR (CDCl₃) δ 11.30 (s, 1 H), 8.75 (dd, J=9.1, 6.6 Hz 1 H), 7.75-6.65 (m, 9 H), 4.15 (m, 2 H), 3.90 (s, 3 H), 3.80 (m, 4 H), 3.55 (m, 2 H); MS m/z (M+1)506.

Step 2: Lithium 2-[(2,4-dichlorobenzoyl)amino]-5-(2-[2-(2-pyridinylamino)ethoxy]ethoxy) benzoate

To a solution of methyl 2-[(2,4-dichlorobenzoyl)amino]-5-{2-[2-(2-pyridinylamino) ethoxy]ethoxy}benzoate (27 mg) in THF (2 ml) was added 2 eq of IM LiOH. The reaction was stirred for as long as need for the hydrolysis to be shown to be complete by TLC assessment. After evaporation of the solvents, the title compound was isolated in a quantitative yield. MS m/z (M−1) 488.

### Example 85

Lithium 5-[2-(2-{[(3-chloro-4-methylphenyl)sulfonyl]amino}ethoxy)ethoxyl-2-[(2,4-dichlorobenzoyl]amino]benzoate

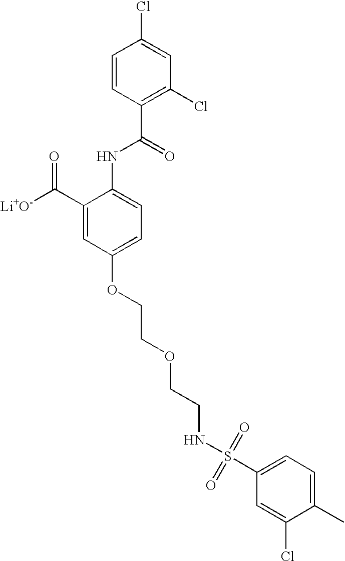

Step 1: Methyl 5-[2-(2-{[(3-chloro-4-methylphenyl)sulfonyljamino~ethoxy)ethoxy]-2-[(2,4-dichlorobenzoyl]amino]benzoate

Use of 3-chloro-4-methylbenzene-1-sulfonyl chloride (47 mg, 0.21 mmol) afforded the title compound (14 mg, 11 %) by the application of the general procedure X described above. MS m/z (M+1) 617.

Step 2: Lithium 5-[2-(2-([(3-chloro-4-methylphenyl)suifonyl]amino~ethoxy)ethoxy]-2-[(2,4-dichlorobenzoyl)amino]benzoate

Use of methyl 5-[2-(2-{[(3-chloro-4-methylphenyl)sulfonyl]amino }ethoxy)ethoxy]-2-[(2,4-dichlorobenzoyl)amino]benzoate (14 mg, 0.22 mmol) afforded the title compound (14 mg, 82%) by the application of the general procedure Y described above. MS m/z (M−1) 601.

### Example 86

Lithium 2-[(2,4-dichlorobenzoyl)amino]-5-(2-12-[(3-pyridinylsulfonyl)amino]ethoxy}ethoxy)benzoate

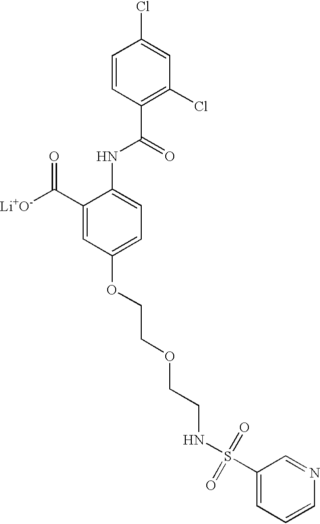

Step 1: Methyl 2-[(2,4-dichlorobenzoyl)amino]-5-(2-(2-[(3-pyridinylsufonyl)amino]ethoxy)ethoxy)benzoate

Use of pyridine-3-sulfonyl chloride (37 mg, 0.21 mmol; Corey, E. J. et al.; J. Org. Chem.; EN; 54; 2; 1989; 389-393) afforded the title compound (8 mg, 6%) by the application of the general procedure X described above. MS m/z (M+1) 568.

Step 2: Lithium 2-[(2,4-dichlorobenzoyl)amino]-5-(2-(2-[(3-pyridinylsulfonyl{amino]ethoxy}ethoxy)benzoate

Use of methyl 2-[(2,4-dichlorobenzoyl)amino]-5-(2-{2-[(3-pyridinylsulfonyl)amino]ethoxy}ethoxy)benzoate (7.2 mg, 0.13 mmol) afforded the title compound (7.7 mg, 84%) by the application of the general procedure Y described above. MS m/z (M−1) 553.

### Example 87

Lithium 2-[(2,4-dichlorobenzoyl)amino]-5-[2-(2-{t(2,4-difluoroanilino)carbonyll amino}ethoxy)ethoxy]benzoate

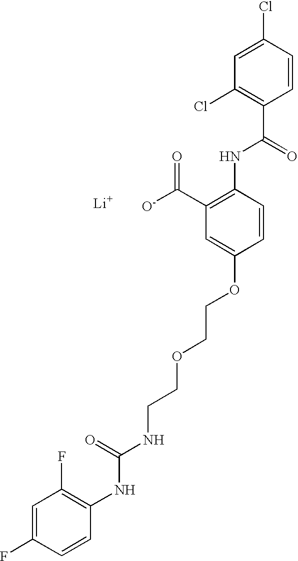

Step 1: Methyl 2-[(2,4-dichlorobenzoyl)amino]-5-[2-(2-{[(2,4-difluoroanilino)carbonyl]amino)ethoxy)ethoxy]benzoate

A suspension of methyl 5-[2-(2-aminoethoxy)ethoxy]-2-[(2,4-dichlorobenzoyl)amino]benzoate trifluoroacetate (90 mg, 0.16 mmol), 2,4-difluorophenyl isocyanate (40 mg, 0.26 mmol) and resin-bound diisopropylethylamine (0.40 mmol) in acetonitrile:DCM (1:1) (6 ml) was agitated at room temperature for 20 hours. After filtration and evaporation the residue was purified by preparative LC-MS to give the title compound (16 mg, 17%). MS m/z (m+1) 582.

Step 2: Lithium 2-[(2,4-dichlorobenzoyl)amino]-5-[2-(2-{[(2,4-difluoroanilino)carbonyl]amino}ethoxy)ethoxy]benzoate

Use of methyl 2-[(2,4-dichlorobenzoyl)amino]-5-[2-(2-{[(2,4-difluoroanilino)carbonyl]amino}ethoxy)ethoxy]benzoate (16 mg, 0.027 mmol) afforded the title compound (17 mg, 97%) by the application of the general procedure Y described above. MS m/z (M−1) 566.

### Example 88

Lithium 2-[(2,4-dichlorobenzoyl)amino]-5-[2-(2-{1[(4-fluoroanilino)carbonyl]amino}ethoxy)ethoxylbenzoate

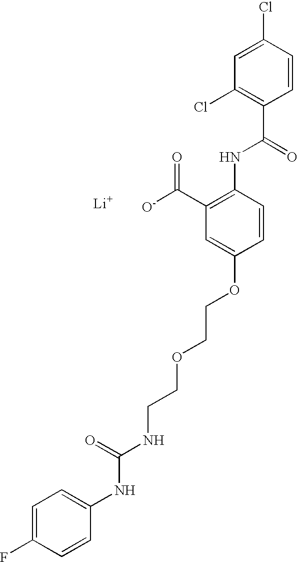

Step 1: Methyl 2-[(2,4-dichlorobenzoyl)amino]-5-[2-(2-([(4-fluoroanilino)carbonyl]amino) ethoxy)ethoxy]benzoate

Use of 4-fluorophenyl isocyanate (29 mg, 0.21 mmol) afforded the title compound (2.1 mg, 2%) by the application of the general procedure X described above. MS m/z (M+1) 564.

Step 2: Methyl 2-[(2,4-dichlorobenzoyl)amino]-5-[2-(2-{[(4-fluoroanilino)carbonyl]amino}ethoxy)ethoxy]benzoate

Use of methyl 2-[(2,4-dichlorobenzoyl)amino]-5-[2-(2-{[(4-fluoroanilino)carbonyl]amino}ethoxy)ethoxy]benzoate (2.1 mg, 0.0037 mmol) afforded the title compound (3.3 mg, 81 %) by the application of the general procedure Y described above. MS m/z (M−1) 548.

### Example 89

Lithium 2-[(2,4-dichlorobenzoyl)amino]-5-12-(2-{[(4-isopropylanilino)carbonyl]amino}ethoxy)ethoxy]benzoate

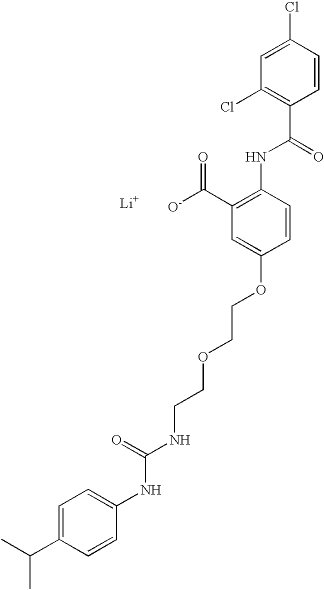

Step 1: Methyl 2-[(2,4-dichlorobenzoyl)amino]-5-[2-(2-([(4-isopropylanilino)carbonyl]amino}ethoxy)ethoxy]benzoate

Use of 4-isopropylphenyl isocyanate (34 mg, 0.21 mmol) afforded the title compound (24 mg, 20%) by the application of the general procedure X described above. MS m/z (M+l) 588.

Step 2: Lithium 2-[(2,4-dichlorobenzoyl)amino]-5-[2-(2-([(4-isopropylanilino)carbonyljaminoyethoxy)ethoxy]benzoate

Use of methyl 2-[(2,4-dichlorobenzoyl)amino]-5-[2-(2-{[(4-isopropylanilino)carbonyl]amino}ethoxy)ethoxy]benzoate (24 mg, 0.041 mmol) afforded the title compound (25 mg, 95%) by the application of the general procedure Y described above. MS m/z (M−1) 572.

### Example 90

Lithium 2-[(2,4-dichlorobenzoyl)amino]-5-12-(methylsulfanyl)ethoxy]benzoate

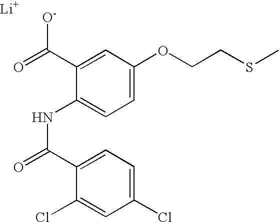

Step 1: Methyl 2-[(2,4-dichlorobenzoyl)amino]-5-[2-(methylsulfanyl)ethoxy]benzoate

Use of 2-(methylsulfanyl)ethanol afforded the title compound (8 mg, 4%), as a white solid, by the application of the general procedure A described above. ¹H NMR (CDCl₃) δ 11.30 (s, 1H), 8.78 (d, J=9.23 Hz 1H), 7.62-7.55 (m, 2H), 7.48 (d, J=2.12 Hz 1H), 7.35 (dd, J=8.31, 1.98 Hz 1H), 4.17 (t, J=6.86 Hz 2H), 3.90 (s, 3H), 2.89 (t, J=6.86Hz 2H), 2.22 (s, 3H); MS 414 m/z (M+1).

Step 2: Lithium 2-[(2,4-dichlorobenzoyl)amino]-5-[2-(methylsulfanyl)ethoxy]benzoate

Use of methyl 2-[(2,4-dichlorobenzoyl)amino]-5-[2-(methylsulfanyl)ethoxy]benzoate afforded the title compound (4.4 mg, 100%) as a white solid by the application of the general procedure B described above. MS m/z 398 (M−1).

Biological Methods

(I) Cell-based reporter assays

The effect of compounds according to the invention on activation of PPARα and PPARγ were determined. Reporter gene assays were performed essentially as described in Bertilsson et al., 1998 (Proc. Natl. Acad. Sci. U.S.A. 95:12208-12213), by transient co-transfections of CaCo2/TC cells with a GAL-4-LBD (Ligand Binding Domain) fusion constructs, containing the nucleotide sequence corresponding to human PPARαLBD (i.e. amino acid residuesl67-468) or human PPARγLBD (i.e. amino acid residues 204-477), together with a 4×UAS-luciferase reporter gene construct, using the FuGENE-6 transfection reagent (Roche) according to the manufacturers recommendations. After 24 hours, the cells were treated with trypsin, transferred to 96-well microplates and allowed to settle. Induction was performed for 24 hours by applying different concentrations of compounds diluted in DMSO or DMSO alone (vehicle). Subsequently, the cells were lysed and luciferase activity measured, according to standard procedures. Experiments were performed in quadruplicate on at least three occasions.

The compounds of formula I exhibit EC₅₀ values on PPARα and PPARγ in the range of 1-35 μM and 0.3-50 μM, respectively.

(II) Ligand binding assays

Crude extracts were prepared from E. coli (BL21(DE3)pLysS, Novagen) producing GST-PPARαLBD or GST-PPARγLBD fusion proteins by freeze thawing in buffer containing 50 mM Tris—HCl pH 7.9, 250 mM KCl, 10% glycerol, 1% Triton X-100, 10 mM DTT, lmM PMSF, 10 [tg/mL DNase and 10 mM MgCl. Competitive ligand binding assays were performed on immobilized GST-PPARαLBD or GST-PPARγLBD fusion proteins from crude extracts incubated with glutathione-Sepharose 4B (Amersham Pharmacia Biotech). Following immobilization, the slurry was washed three times in binding buffer containing 50 mM Tris-HCL, pH7.9, 50 mM KCl, 0.1% Triton-X100, 10 mM DTT, 2 mM EDTA, dispensed in 96-well filter plates (MHVB N45, Millipore) and incubated with a fixed amount tritiated ligand and different concentrations of cold competing ligands. Equilibrium binding was reached after incubation for 2 hours at room temperature on a plate shaker. The plates were then washed 3 times in binding buffer, dried overnight at room temperature followed by scintillation counting after the addition of 25 μl of scintillant (Optiscint Hisafe, Wallac) per well. Each experiment was performed in duplicate and repeated independently at least three times. 3H-BRL49653 (ART-605; American Radiolabeled Chemicals, USA) was used as the tracer in PPARγ competitive ligand binding experiments at a concentration of 30 nM (10). ³H-GW2331 (70 μM) was synthesized at Pharmacia Corporation and used as the tracer in PPAR(X competitive ligand binding experiments (Kliewer, S. A. et al. (1997) Proc. Natl. Acad. Sci. U.S.A. 94: 4318-4323).

The compounds of formula I exhibit Kᵢ values on PPARα and PPARγ in the range of 1-70 μM and 0.3-35 μM, respectively.

(III) In vivo experiment

Selected compounds of formula I were tested in animal models of relevance for measuring PPARγ efficacy. The animal model used was ob/ob mouse and as a reference compound the known PPARγ ligand, rosiglitazone. The animals were orally treated during 7 days and parameters as food intake, body weight, plasma glucose, insulin, cholesterol, triglycerides and free fatty acids were monitored. Compounds of formula I were shown to give dose related pharmacological effects.

Structural Studies of a Representative from the Compound Series

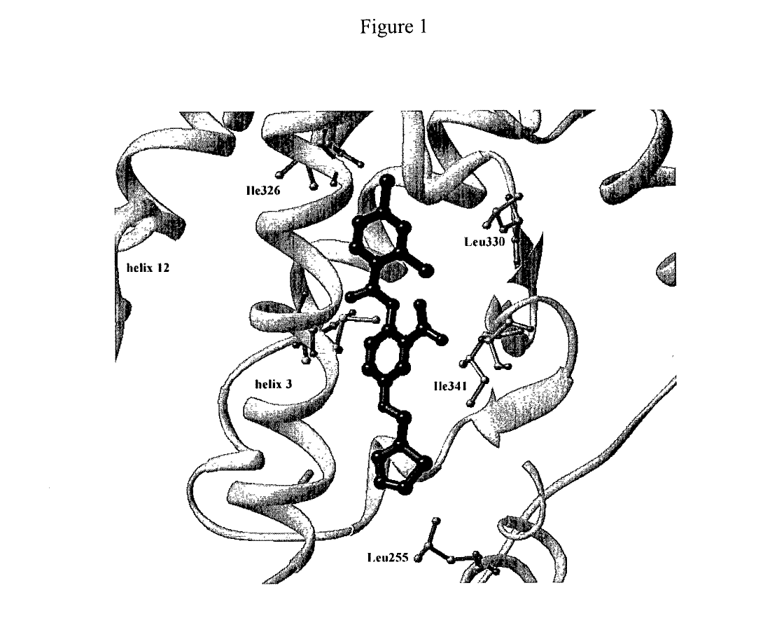

The structure of PPARγ ligand binding domain (LBD) has previously been described in literature (Nolte, R. T. et al. (1998) Nature 395: 137-143; Uppenberg, J. et al. (1998) J. Biol. Chem. 273: 31108-31112). The present inventors have determined the structure of human PPARγ LBD in complex with one of the compounds (Example 1) according to the invention. As indicated in FIG. 1, the compound according to Example 1 was shown to be located in the ligand binding pocket of human PPARγ. The compound was found in an elongated conformation and occupied a region in proximity with, and approximately parallel to, helix 3 (the numbering of helices and strands follow the convention of Uppenberg et al. supra) and in proximity to beta-strand 3 and helices 5 and 2b.

The interactions between the compound (ligand) according to the invention and human PPARγ can be separated into four categories:

(1) Interaction between the dibromo-phenyl moiety of the ligand and the predominantly hydrophobic pocket of human PPARγ, in particular the side chains of Ile326, Met329, Leu330, Leu333, Ala292 and Arg288.

(2) Interaction between the carbonyl oxygen on the peptide linker of the ligand and the side chain sulfur, as well as the backbone carbonyl oxygen, of Cys285 in human PPARγ.

(3) Interaction between the central benzoic acid moiety of the ligand and human PPARγ. The central benzoic acid moiety is located in a narrow groove in the protein made up by the side chains of Met364, Ile341, Cys285 and Arg288, but also the backbone atoms of Cys285, Ala284 and Ser342. The interactions are hydrophobic in nature, with the exception of a distinct hydrogen bond formed between the backbone nitrogen of Ser342 and one of the carboxylate oxygens of the ligand.

(4) Interaction between the thiophene tail of the ligand and human PPARγ. This interaction is predominantly hydrophobic in nature. The protein atoms involved belong to the side chains and backbone of Leu255, Ile281 and Arg280.

It has been argued that the activation of nuclear receptors, including PPARγ, follows a common mechanism where a ligand binds in the ligand binding pocket and thereby stabilizes helix 12, which in turn allows for the recruitment of coactivator proteins and subsequent activation of the transcriptional machinery. Furthermore it has been reported (Elbrecht, A. et al. (1999) J. Biol. Chem. 274: 7913-7922) that Cys 285 on helix 3 is also important for the induction of conformational changes mediating this mechanism. Ligands, binding in different parts of the binding pocket, have been reported to show varying and tissue specific agonistic effects from full to partial agonism, as illustrated by the estrogen receptor (McDonnell, D. P. et al. (1995) Mol. Endocrinol. 9: 659-669; Mueller-Fahrnow, A. and Egner, U. (1999) Current opinion in Biotechnology 10: 550-556). Compared with previously disclosed PPAR ligands, the compounds according to the invention bind in a novel binding mode. These compounds modulate the activity of PPARs in a range of agonistic effects determined in a cell based reporter assay.

What is claimed is:

1. A compound of the formula I 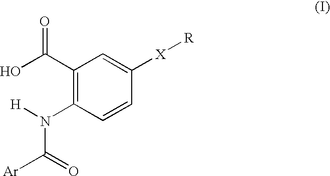
or a pharmaceutically acceptable salt or a prodrug form thereof, wherein
Ar is aryl, which is optionally substituted in one or more positions by
halogen,
cyano,
nitro,
C₁₋₆alkyl,
C₁₋₆alkoxy,
C₁₋₆alkylthio
fluoromethyl,
difluoromethyl,
trifluoromethyl,
difluoromethoxy,
trifluoromethoxy,
difluoromethylthio,
trifluoromethylthio,
allyloxy,
aryloxy, or
arylthio;
X is
a bond,
a heteroalkyl chain comprising from 1 to 4 carbon atoms and from 1 to 4 heteroatoms,
or
a formula 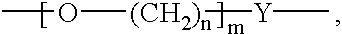
wherein m is 0, 1, or 2,
n is 0, 1, 2, or 3, and
Y is a bond, O, S, NH, NHSO₂, NHC(O)NH, or CH═CH; and
R is a C₁-C₆-alkyl or an optionally substituted aryl or heteroaryl group, with the proviso that when X is a bond or O, then R is not a C₁-C₆-alkyl; or
that said compound is not a dibenzoyl-bisanthranilic acid, or (4,4′-bis[(1-naphthalenylcarbonyl)amino]-[1,1′-Biphenyl]-3,3′-dicarboxylic acid.

2. The compound according to claim 1 wherein Ar is an, optionally substituted, phenyl or naphthyl.

3. The compound according to claim 2 wherein Ar is phenyl, substituted in one or more positions independently by halogen, nitro, cyano, methoxy, or trifluoromethyl.

4. The compound according to claim 3 wherein the said phenyl group is substituted in one or more positions by halogen.

5. The compound according to claim 4 wherein Ar is 2,4-dichlorophenyl.

6. The compound according to claim 1 wherein X is
O—(CH₂)ₙ—,
O—(CH₂)ₙ—Y— and Y is an atom selected from O, N and S,
O—(CH₂)₂—O—(CH₂)₂—NH,
O—(CH₂)₂—O—(CH₂)₂—NHSO₂, or
O—(CH₂)₂—O—(CH₂)₂—NHCONH.

7. The compound according to claim 6 wherein X is
O,
O—CH₂,
O—(CH₂)₂,
O—(CH₂)₂—O, or
O—(CH₂)₂—S.

8. The compound according to claim 1 wherein X is a bond.

9. The compound according to claim 1 wherein R is an optionally substituted aryl or heteroaryl group.

10. The compound according to claim 9 wherein R is selected from the group consisting of, optionally substituted, phenyl, naphthyl, thienyl, pyridinyl, quinoxalinyl, benzoylphenyl, thiazolyl, furyl, imidazolyl, oxazolyl, pyrazinyl, quinolinyl, indolyl, benzofuran, benzothiophenyl (benzothienyl), pyrimidinyl, benzodioxolyl.

11. The compound according to claim 10 wherein the group R is independently substituted in one or more positions with
C₁₋₆-alkyl,
C₁₋₆-alkoxy,
C₁₋₆-alkylthio,
C₁₋₆-acyl,
cyano,
nitro,
hydroxy,
methylhydroxy,
carboxy,
fluoromethyl,
difluoromethyl,
trifluoromethyl,
difluoromethoxy,
trifluoromethoxy,
difluoromethylthio,
trifluoromethylthio,
halogen,
formyl,
amino,
C₁₋₆-alkylamino,
di(C₁₋₆-alkyl)amino or C₁₋₆-acylamino,
aryl,
aryloxy,
arylthio,
C₁₋₆-alkylsulphonyl,
C₂₋₆-allyloxy,
benzyloxy,
benzoyl.

12. The compound according to claim 11 wherein R is independently substituted in one or more positions with
methyl,
ethyl,
isopropyl,
methoxy,
thiomethoxy
ethoxy,
methylsulfonyl,
formyl,
acetyl,
nitro,
cyano,
methylhydroxy,
methylamino,
carboxy,
trifluoromethyl,
trifluoromethoxy,
chloro,
fluoro,
bromo,
iodo,
benzyloxy,
amino,
dimethylamino,
acetylamino,
phenyl, or
phenoxy,
benzoyl.

13. The compound according to claim 1 wherein R is methyl.

14. The compound according to claim 1 which is the compound
2-[(2,4-dichlorobenzoyl)amino]-5-(2-thienylmethoxy)benzoate,
2-[(2,4-dichlorobenzoyl)amino]-5-(3-pyridinylmethoxy)benzoate,
2-[(2,4-dichlorobenzoyl)amino]-5-[2-(3-thienyl)ethoxy]benzoate,
5-[2-(3-chlorophenyl)ethoxy]-2-[(2,4-dichlorobenzoyl)amino]benzoate,
2-[(2,4-dichlorobenzoyl)amino]-5-[(4-ethoxybenzyl)oxy]benzoate,
2-[(2,4-dichlorobenzoyl)amino]-5-{[3-(dimethylamino)benzyl]oxy}benzoate,
2-[(2,4-dichlorobenzoyl)amino]-5-[2-(3-methylphenyl)ethoxy]benzoate,
2-[(2,4-dichlorobenzoyl)amino]-5-[2-( 1-naphthyl)ethoxy]benzoate,
2-[(2,4-dichlorobenzoyl)amino]-5-(2-pyridinylmethoxy)benzoate,
2-[(2,4-dichlorobenzoyl)amino]-5-(2-{[2-(methylsulfanyl)-3-pyridinyl]oxy}ethoxy)benzoate,
2-[(2,4-dichlorobenzoyl)amino]-5-[2-(3-pyridinyloxy)ethoxy]benzoate,
2-[(2,4-dichlorobenzoyl)amino]-5-[2-(2-quinoxalinyloxy)ethoxy]benzoate,
2-[(2,4-dichlorobenzoyl)amino]-5-{2-[2-(methylsulfanyl)phenoxy}ethoxy]benzoate,
5-[2-(2-aminophenoxy)ethoxy]-2-[(2,4-dichlorobenzoyl)amino]benzoate,
5-[2-(4-benzoylphenoxy)ethoxy]-2-[(2,4-dichlorobenzoyl)amino]benzoate,
2-[(2,4-dichlorobenzoyl)amino]-5-[2-(2,3,6-trifluorophenoxy)ethoxy]benzoate,
5-[2-([1,1′-biphenyl]-3-yloxy)ethoxy]-2-[(2,4-dichlorobenzoyl)amino]benzoate,
2-[(2,4-dichlorobenzoyl)amino]-5-[2-(phenylsulfanyl)ethoxy]benzoate,
2-[(2,4-dichlorobenzoyl)amino]-5-[2-(4-methyl-1,3-thiazol-5-yl)ethoxy]benzoate,
2-[(2,4-dichlorobenzoyl)amino]-5-(3-furylmethoxy)benzoate,
2-[(2,4-dichlorobenzoyl)amino]-5-[2-(2-thienyl)ethoxy]benzoate,
2-[(2,4-dichlorobenzoyl)amino]-5-[2-(2-methyl-5-nitro-1 H-imidazol-1-yl)ethoxy]benzoate,
2-[(2,4-dichlorobenzoyl)amino]-5-(3-thienylmethoxy)benzoate,
2-[(2,4-dichlorobenzoyl)amino]-5-[2-(2-pyfidinylsulfanyl)cthoxy]benzoate,
2-[(2,4-dichlorobenzoyl)amino]-5-[2-(5-methyl-2-phenyl-1,3-thiazol-4-yl)ethoxy]benzoate,
2-[(2,4-dichlorobenzoyl)amino]-5-[2-(5-methyl-2-phenyl-1 ,3-oxazol-4-yl)ethoxy]benzoate,
2-[(2,4-dichlorobenzoyl)amino]-5-[2-(5-ethyl-2-pyridinyl)ethoxy]benzoate,
2-[(2,4-dichlorobenzoyl)amino]-5-[2-(2-methoxyphenoxy)ethoxy]benzoate,
2-[(2,4-dichlorobenzoyl)amino]-5-(4-pyridinylmethoxy)benzoate,
2-[(2,4-dichlorobenzoyl)amino]-5-[3-(3-pyridinyl)propoxy]benzoate,
2-[(2,4-dichlorobenzoyl)amino]-5-[2-(2-pyridinyl)ethoxy]benzoate,
2-[(2,4-dichlorobenzoyl)amino]-5-[2-(3-methoxyphenyl)ethoxy]benzoate,
2-[(2,4-dichlorobenzoyl)amino]-5-[(3-nitro-2-pyridinyl)oxy]benzoate,
2-[(2,4-dichlorobenzoyl)amino]-5-[(5-nitro-2-pyridinyl)oxy]benzoate,
2-[(2,4-dichlorobenzoyl)amino]-5-{[5-(trifluoromethyl)-2-pyridinyl]oxy}benzoate,
5-[(6-chloro-2-pyrazinyl)oxy]-2-[(2,4-dichlorobenzoyl)amino]benzoate,
2-[(2,4-dichlorobenzoyl)amino]-5-(2-pyrimidinyloxy)benzoate,
2-[(2,4-dichlorobenzoyl)amino]-5-{[(2E)-3-phenyl-2-propenyl]oxy}benzoate,
2-[(2,4-dichlorobenzoyl)amino]-5-[(3-methoxybenzyl)oxy]benzoate,
2-[(2,4-dichlorobenzoyl)amino]-5-(3-thienyl)benzoate,
2-[(2,4-dichlorobenzoyl)amino]-5-(2,4-dichlorophenyl)benzoate,
2-[(2,4-dichlorobenzoyl)amino]-5-(4-ethylphenyl)benzoic acid,
2-[(2,4-dichlorobenzoyl)amino]-5-(8-quinolinyl)benzoic acid,
5-(1,3-benzodioxol-5-yl)-2-[(2,4-dichlorobenzoyl)amino]benzoic acid,
2-[(2,4-dichlorobenzoyl)amino]-5-(2,4-dimethoxy-5-pyrmidinyl)benzoic acid,
3′-(acetylamino)-4-[(2,4-dichlorobenzoyl)amino][1,1′-biphenyl]-3-carboxylic acid,
4-[(2,4-dichlorobenzoyl)amino]-3′-(trifluoromethoxy)[1,1′-biphenyl]-3-carboxylic acid,
4-[(2,4-dichlorobenzoyl)amino]-3′-ethoxy[1,1′-biphenyl]-3-carboxylic acid,
5-(1-benzofuran-2-yl)-2-[(2,4-dichlorobenzoyl)amino]benzoic acid,
4-[(2,4-dichlorobenzoyl)amino]-3′-(hydroxymethyl)[1,1′-biphenyl]-3-carboxylic acid,
4-[(2,4-dichlorobenzoyl)amino]-3′-formyl[1,1′-biphenyl]-3-carboxylic acid,
2-[(2,4-dichlorobenzoyl)amino]-5-(2-naphthyl)benzoic acid,
4-[(2,4-dichlorobenzoyl)amino]-3′-isopropyl-6′-methoxy[1,1′-biphenyl]-3-carboxylic acid,
4-[(2,4-dichlorobenzoyl)amino]-4′-fluoro[1,1′-biphenyl]-3-carboxylic acid,
2-[(2,4-dichlorobenzoyl)amino]-5-(2-furyl)benzoate,
5-(1-benzothien-3-yl)-2-[(2,4-dichlorobenzoyl)amino]benzoate,
2-[(2,4-dichlorobenzoyl)amino]-5-(2-formyl-3-thienyl)benzoate,
5-(5-acetyl-2-thienyl)-2-[(2,4-dichlorobenzoyl)amino]benzoate,
2-[(2,4-dichlorobenzoyl)amino]-5-(1 H-indol-5-yl)benzoic acid,
5-(3-carboxyphenyl)-2-[(2,4-dichlorobenzoyl)amino]benzoic acid,
2′-(benzyloxy)-4-[(2,4-dichlorobenzoyl)amino][1,1′-biphenyl]-3-carboxylate,
4-[(2,4-dichlorobenzoyl)amino][1,1′-biphenyl]-3-carboxylate,
4-[(2,4-dichlorobenzoyl)amino]-3′-phenyl[1,1′-biphenyl]-3-carboxylate,
4-[(2,4-dichlorobenzoyl)amino]-3′-(trifluoromethyl)[1,1′-biphenyl]-3-carboxylate,
5-(5-chloro-2-thienyl)-2-[(2,4-dichlorobenzoyl)amino]benzoate,
4-[(2,4-dichlorobenzoyl)amino]-4′-phenoxy[1,1′-biphenyl]-3-carboxylate,
4-[(2,4-dichlorobenzoyl)amino]-2′,5′-dimethoxy[1,1′-biphenyl]-3-carboxylate,
3′-(aminomethyl)-4-[(2,4-dichlorobenzoyl)amino][1,1′-biphenyl]-3-carboxylic acid,
2-(2-naphthoylamino)-5-(3-thienyl)benzoate,
3′-(acetylamino)-4-(2-naphthoylamino)[1,1′-biphenyl]-3-carboxylate
3′-(hydroxymethyl)-4-(2-naphthoylamino)[1,1′-biphenyl]-3-carboxylate,
5-(3-thienyl)-2-{[4-(trifluoromethyl)benzoyl]amino}benzoate,
3′-(acetylamino)-4-{[4-(trifluoromethyl)benzoyl]amino}[1,1′-biphenyl]-3-carboxylate,
3′-(hydroxymethyl)-4-{[4-(trifluoromethyl)benzoyl]amino}[1,1′-biphenyl]-3-carboxylate,
2-{[3,5-bis(trifluoromethyl)benzoyl]amino}-5-(8-quinolinyl)benzoate,
4-{[3,5-bis(trifluoromethyl)benzoyl]amino}-3′-formyl[1,1′-biphenyl]-3-carboxylate,
2-[(4-methoxybenzoyl)amino]-5-(8-quinolinyl)benzoate,
4-[(3 ,5-dichlorobenzoyl)amino][1,1′-biphenyl]-3-carboxylate,
2-[(4-cyanobenzoyl)amino]-5-(2-thienylmethoxy)benzoate,
2-[(2,4-difluorobenzoyl)amino]-5-(2-thienylmethoxy)benzoate,
2-[(2-chlorobenzoyl)amino]-5-[(3-nitro-2-pyridinyl)oxy]benzoate,
2-[(2-chloro-5-nitrobenzoyl)amino]-5-(2-thienylmethoxy)benzoate, or
2-[(2,4-dichlorobenzoyl)amino]-5-[2-(methylsulfanyl)ethoxy]benzoate.

15. A pharmaceutical formulation containing a compound according to claim 1 as an active ingredient in combination with a pharmaceutically acceptable diluent or carrier.

16. A method for treatment or prevention of diabetes, comprising administering to a subject in need thereof an effective amount of a compound according to the formula I 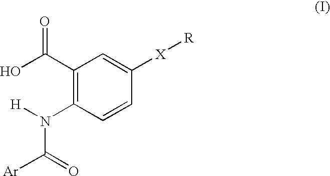
or a pharmaceutically acceptable salt or a prodrug form thereof, wherein
Ar is aryl, which is optionally substituted in one or more positions by
halogen,
cyano,
nitro,
C₁₋₆alkyl,
C₁₋₆alkoxy,
C₁₋₆alkylthio
fluoromethyl,
difluoromethyl,
trifluoromethyl,
difluoromethoxy,
trifluoromethoxy,
difluoromethylthio,
trifluoromethylthio,
allyloxy,
aryloxy, or
arylthio;
X is
a bond, or
a heteroalkyl chain comprising from 1 to 4 carbon atoms and from 1 to 4 heteroatoms,
or
a formula 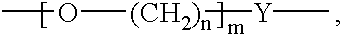
wherein m is 0, 1, or 2,
n is 0, 1, 2, or 3, and
Y is a bond, O, S, NH, NHSO₂, NHC(O)NH, or CH═CH; and
R is C₁-C₆-alkyl or an optionally substituted aryl or heteroaryl group.

17. The method according to claim 16, wherein the said compound is
2-[(2,4-dichlorobenzoyl)amino]-5-(2-thi enylmethoxy)b enzo ate,
2-[(2,4-dichlorobenzoyl)amino]-5-(3-pyridinylmethoxy)benzoate,
2-[(2,4-dichlorobenzoyl)amino]-5-[2-(3-thienyl)ethoxy]benzoate,
5-[2-(3-chlorophenyl)ethoxy]-2-[(2,4-dichlorobenzoyl)amino]benzoate,
2-[(2,4-dichlorobenzoyl)amino]-5-[(4-ethoxybenzyl)oxy]benzoate,
2-[(2,4-dichlorobenzoyl)amino]-5-{[3-(dimethylamino)benzyl]oxy}benzoate,
2-[(2,4-dichlorobenzoyl)aamino]-5-[2-(3-methylphenyl)ethoxy]benzoate,
2-[(2,4-dichlorobenzoyl)amino]-5-[2-(1-naphthyl)ethoxy]benzoate,
2-[(2,4-dichlorobenzoyl)amino]-5-(2-pyridinylmethoxy)benzoate,
2-[(2,4-dichlorobenzoyl)amino]-5-(2-{[2-(methylsulfanyl)-3-pyridinyl]oxy}ethoxy)benzoate,
2-[(2,4-dichlorobenzoyl)amino]-5-[2-(3-pyridinyloxy)ethoxy]benzoate,
2-[(2,4-dichlorobenzoyl)amino]-5-[2-(2-quinoxalinyloxy)ethoxy]benzoate,
2-[(2,4-dichlorobenzoyl)amino]-5-{2-[2-(methylsulfanyl)phenoxy]ethoxy}benzoate,
5-[2-(2-aminophenoxy)ethoxy]-2-[(2,4-dichlorobenzoyl)amino]benzoate,
5-[2-(4-benzoylphenoxy)ethoxy]-2-[(2,4-dichlorobenzoyl)amino]benzoate,
2-[(2,4-dichlorobenzoyl)amino]-5-[2-(2,3,6-trifluorophenoxy)ethoxy]benzoate,
5-[2-([1,1′-biphenyl]-3-yloxy)ethoxy]-2-[(2,4-dichlorobenzoyl)amino]benzoate,
2-[(2,4-dichlorobenzoyl)amino]-5-[2-(phenylsulfanyl)ethoxy]benzoate,
2-[(2,4-dichlorobenzoyl)amino]-5-[2-(4-methyl-1,3-thiazol-5-yl)ethoxy]benzoate,
2-[(2,4-dichlorobenzoyl)amino]-5-(3-furylmethoxy)benzoate,
2-[(2,4-dichlorobenzoyl)amino]-5-[2-(2-thienyl)ethoxy]benzoate,
2-[(2,4-dichlorobenzoyl)amino]-5-[2-(2-methyl-5-nitro-1H-imidazol-1-yl)ethoxy]benzoate,
2-[(2,4-dichlorobenzoyl)amino]-5-(3-thienylmethoxy)benzoate,
2-[(2,4-dichlorobenzoyl)amino]-5-[2-(2-pyridinylsulfanyl)ethoxy]benzoate,
2-[(2,4-dichlorobenzoyl)amino]-5-[2-(5-methyl-2-phenyl-1,3-thiazol-4-yl)ethoxy]benzoate,
2-[(2,4-dichlorobenzoyl)amino]-5-[2-(5-methyl-2-phenyl-1,3-oxazol-4-yl)ethoxy]benzoate,
2-[(2,4-dichlorobenzoyl)amino]-5-[2-(5-ethyl-2-pyridinyl)ethoxy]benzoate,
2-[(2,4-dichlorobenzoyl)amino]-5-[2-(2-methoxyphenoxy)ethoxy]benzoate,
2-[(2,4-dichlorobenzoyl)amino]-5-(4-pyridinylmethoxy)benzoate,
2-[(2,4-dichlorobenzoyl)amino]-5-[3-(3-pyridinyl)propoxy]benzoate,
2-[(2,4-dichlorobenzoyl)amino]-5-[2-(2-pyridinyl]ethoxy]benzoate,
2-[(2,4-dichlorobenzoyl)amino]-5-[2-(3-methoxyphenyl)ethoxy]benzoate,
2-[(2,4-dichlorobenzoyl)amino]-5-[(3-nitro-2-pyridinyl)oxy]benzoate,
2-[(2,4-dichlorobenzoyl)amino]-5-[(5-nitro-2-pyridinyl)oxy]benzoate,
2-[(2,4-dichlorobenzoyl)amino]-5-{[5-(trifluoromethyl)-2-pyridinyl]oxy}benzoate,
5-[(6-chloro-2-pyrazinyl)oxy]-2-[(2,4-dichlorobenzoyl)amino]bcnzoate,
2-[(2,4-dichlorobenzoyl)amino]-5-(2-pyrimidinyloxy)benzoate,
2-[(2,4-dichlorobenzoyl)amino]-5-{[(2E)-3-phenyl-2-propenyl]oxy}benzoate,
2-[(2,4-dichlorobenzoyl)aamino]-5-[(3-methoxybenzyl)oxy]benzoate,
2-[(2,4-dichlorobenzoyl)amino]-5-(3-thienyl)benzoate,
2-[(2,4-dichlorobenzoyl)amino]-5-(2,4-dichlorophenyl)benzoate,
2-[(2,4-dichlorobenzoyl)amino]-5-(4-ethylphenyl)benzoic acid,
2-[(2,4-dichlorobenzoyl)amino]-5-(8-quinolinyl)benzoic acid,
5-(1,3-benzodioxol-5-yl)-2-[(2,4-dichlorobenzoyl)amino]benzoic acid,
2-[(2,4-dichlorobenzoyl)amino]-5-(2,4-dimethoxy-5-pyrimidinyl)benzoic acid,
3′-(acetylamino)-4-[(2,4-dichlorobenzoyl)amino][1,1′-biphenyl]-3-carboxylic acid,
4-[(2,4-dichlorobenzoyl)amino]-3′-(trifluoromethoxy)[1,1′-biphenyl]-3-carboxylic acid,
4-[(2,4-dichlorobenzoyl)amino]-3′-ethoxy[1,1′-biphenyl]-3-carboxylic acid,
5-(1-benzofuran-2-yl)-2-[(2,4-dichlorobenzoyl)amino]benzoic acid,
4-[(2,4-dichlorobenzoyl)amino]-3′-(hydroxymethyl)[1,1′-biphenyl]-3-carboxylic acid,
4-[(2,4-dichlorobenzoyl)amino]-3′-fonnyl[1,1′-biphenyl]-3-carboxylic acid,
2-[(2,4-dichlorobenzoyl)amino]-5-(2-naphthyl)benzoic acid,
4-[(2,4-dichlorobenzoyl)amino]-3′-isopropyl-6′-methoxy[1,1′-biphenyl]-3-carboxylic acid,
4-[(2,4-dichlorobenzoyl)amino]-4′-fluoro[1,1′-biphenyl]-3-carboxylic acid,
2-[(2,4-dichlorobenzoyl)amino]-5-(2-furyl)benzoate,
5-( 1-benzothien-3-yl)-2-[(2,4-dichlorobenzoyl)amino]benzoate,
2-[(2,4-dichlorobenzoyl)amino]-5-(2-formyl-3-thienyl)benzoate,
5-(5-acetyl-2-thienyl)-2-[(2,4-dichlorobenzoyl)amino]benzoate,
2-[(2,4-dichlorobenzoyl)amino]-5-(1 H-indol-5-yl)benzoic acid,
5-(3-carboxyphenyl)-2-[(2,4-dichlorobenzoyl)amino]benzoic acid,
2′-(benzyloxy)-4-[(2,4-dichlorobenzoyl)amino][1,1′-biphenyl]-3-carboxylate,
4-[(2,4-dichlorobenzoyl)amino][1,1′-biphenyl]-3-carboxylate,
4-[(2,4-dichlorobenzoyl)amino]-3′-phenyl[1,1′-biphenyl]-3-carboxylate,
4-[(2,4-dichlorobenzoyl)amino]-3′-(trifluoromethyl)[1,1′-biphenyl]-3-carboxylate,
5-(5-chloro-2-thienyl)-2-[(2,4-dichlorobenzoyl)amino]benzoate,
4-[(2,4-dichlorobenzoyl)amino]-4′-phenoxy[1,1′-biphenyl]-3-carboxylate,
4-[(2,4-dichlorobenzoyl)amino]-2′,5′-dimethoxy[1,1′-biphenyl]-3-carboxylate,
3′-(aminomethyl)-4-[(2,4-dichlorobenzoyl)amino][1,1′-biphenyl]-3-carboxylic acid,
2-(2-naphthoylamino)-5-(3-thienyl)benzoate,
3′-(acetylamino)-4-(2-naphthoylamino)[1,1′-biphenyl]-3-carboxylate
3′-(hydroxymethyl)-4-(2-naphthoylamino)[1,1′-biphenyl]-3-carboxylate,
5-(3-thienyl)-2-{[4-(trifluoromethyl)benzoyl]amino}benzoate,
3′-(acetylamino)-4-{[4-(trifluoromethyl)benzoyl]amino}[1,1′-biphenyl]-3-carboxylate,
3′-(hydroxymethyl)-4-{[4-(trifluoromethyl)benzoyl]amino }[1,1′-biphenyl]-3-carboxylate,
2-{[3,5-bis(trifluoromethyl)benzoyl]amino}-5-(8-quinolinyl)benzoate,
4-{[3,5-bis(trifluoromethyl)benzoyl]amino}-3′-formyl[1,1′-biphenyl]-3-carboxylate,
2-[(4-methoxybenzoyl)amino]-5-(8-quinolinyl)benzoate,
4-[(3 ,5-dichlorobenzoyl)amino][1,1′-biphenyl]-3-carboxylate,
2-[(4-cyanobenzoyl)amino]-5-(2-thienylmethoxy)benzoate,
2-[(2,4-difluorobenzoyl)amino]-5-(2-thienylmethoxy)benzoate,
2-[(2-chlorobenzoyl)amino]-5-[(3-nitro-2-pyridinyl)oxy]benzoate,
2-[(2-chloro-5-nitrobenzoyl)amino]-5-(2-thienylmethoxy)benzoate,
2-[(2,4-dichlorobenzoyl)amino]-5-[2-(methylsulfanyl)ethoxy]benzoate, or
2-[(2,4-dichlorobenzoyl)amino]-m-toluate.

18. A method for modulating peroxisome proliferator-activated receptor activity, comprising contacting the receptor with an effective stimulatory or inhibitory amount of a compound of the formula I: 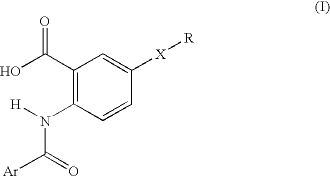
or a pharmaceutically acceptable salt or a prodrug form thereof, wherein
Ar is aryl, which is optionally substituted in one or more positions by
halogen,
cyano,
nitro,
C₁₋₆alkyl,
C₁₋₆alkoxy,
C₁₋₆alkylthio
fluoromethyl,
difluoromethyl,
trifluoromethyl,
difluoromethoxy,
trifluoromethoxy,
difluoromethylthio,
trifluoromethylthio,
allyloxy,
aryloxy, or
arylthio;
X is
a bond, or
a heteroalkyl chain comprising from 1 to 4 carbon atoms and from 1 to 4 heteroatoms,
or
a formula 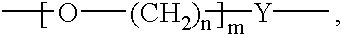
wherein m is 0, 1, or 2,
n is 0, 1,2, or 3, and
Y is a bond, O, S, NH, NHSO₂, NHC(O)NH, or CH═CH; and
R is C₁-C₆-alkyl or an optionally substituted aryl or heteroaryl group.
